(19) United States
(12) Patent Application Publication
(10) Pub. No.: US 2025/0120969 A1
(43) Pub. Date: Apr. 17, 2025

(54) METHODS AND COMPOSITIONS FOR TREATING INFLAMMATION INJURY IN THE LUNGS

(71) Applicant: CalciMedica, Inc., La Jolla, CA (US)

(72) Inventors: Kenneth A. STAUDERMAN, La Jolla, CA (US); Michael DUNN, La Jolla, CA (US); Sudarshan HEBBAR, La Jolla, CA (US); Rachel LEHENY, La Jolla, CA (US)

(21) Appl. No.: 18/777,901

(22) Filed: Jul. 19, 2024

Related U.S. Application Data
(63) Continuation of application No. PCT/US2023/060930, filed on Jan. 19, 2023.
(60) Provisional application No. 63/301,276, filed on Jan. 20, 2022.

(51) Int. Cl. A61K 31/4965; A61K 31/573; A61K 39/395; A61P 11/00

(52) U.S. Cl. CPC A61K 31/4965; A61K 31/573; A61K 39/3955; A61P 11/00

(57) ABSTRACT

Compositions and methods related to the treatment of acute lung injury (ALI) and acute respiratory distress syndrome (ARDS) including viral pneumonia through the pharmaceutical manipulation of calcium signaling are disclosed. Such compositions and methods may be used to reduce pro-inflammatory cytokine releases that may lead to ALI and/or ARDS.

### CROSS REFERENCE

This application is a continuation of International Application No. PCT/US2023/060930, filed Jan. 19, 2023, which claims the benefit of U.S. provisional patent application Ser. No. 63/301,276, filed Jan. 20, 2022, which is incorporated herein by reference in its entirety.

### BACKGROUND

Acute lung injury (ALI) and the acute respiratory distress syndrome (ARDS) describe clinical syndromes of acute respiratory failure with substantial morbidity and mortality. Under the current pandemic of COVID-19, for patients infected with SARS-CoV-2, their immune responses can lead to a “cytokine storm”, which in turn can cause ALI, ARDS, death, or in the case of survivors permanently compromised pulmonary function.

### SUMMARY OF THE INVENTION

In an aspect, the disclosure provides a method for treating acute lung injury (ALI), acute respiratory distress syndrome (ARDS), COVID-19 pneumonia, bacterial pneumonia, viral pneumonia, acute pancreatitis, or a combination thereof, in a subject, comprising administering a therapeutically effective amount of an intracellular Calcium signaling inhibitor to said subject, wherein the subject has a PaO₂/FiO₂ (P/F) ratio of less than or equal to about 150, and wherein the subject has been administered with at least two doses of the intracellular Calcium signaling inhibitor.

In another aspect, the disclosure provides a method for treating acute lung injury (ALI), acute respiratory distress syndrome (ARDS), COVID-19 pneumonia, bacterial pneumonia, viral pneumonia, acute pancreatitis, or a combination thereof, in a subject, comprising administering a therapeutically effective amount of a corticosteroid and/or immunosuppressive drug and an intracellular Calcium signaling inhibitor to said subject, wherein the subject has a PaO₂/FiO₂ (P/F) ratio of less than or equal to about 150.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a chart showing an example of a clinical trial design for evaluating CRAC inhibitors in patients with severe COVID-19 pneumonia.

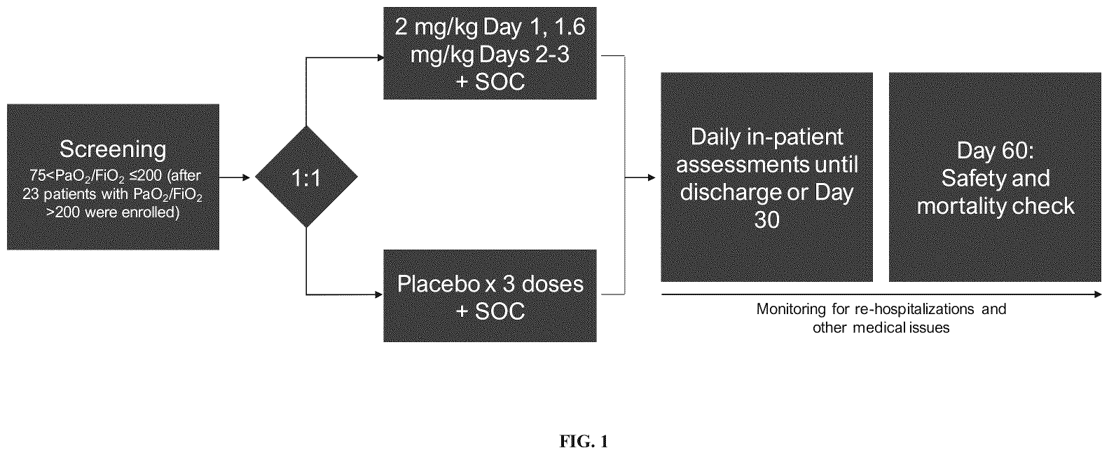

FIG. 2 is a graph showing the mortality of patients at Day 30 and Day 60 for patients treated with placebo or Auxora.

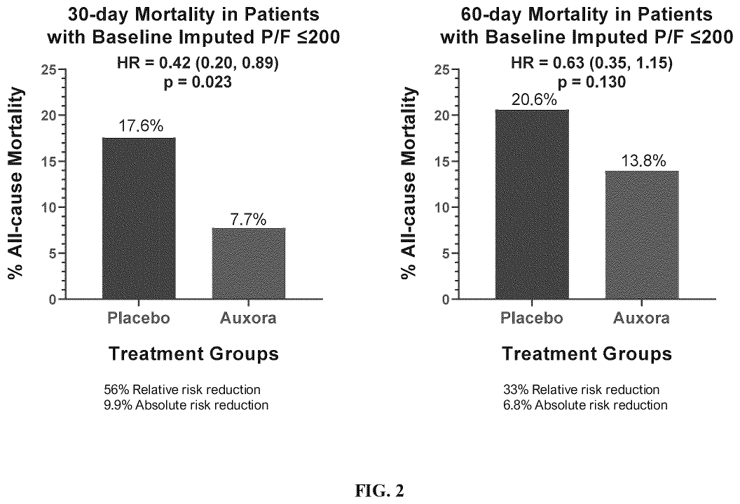

FIG. 3 is a plot showing mortality through Day 60 using KM Efficacy Analysis Set with patients treated with placebo or Auxora.

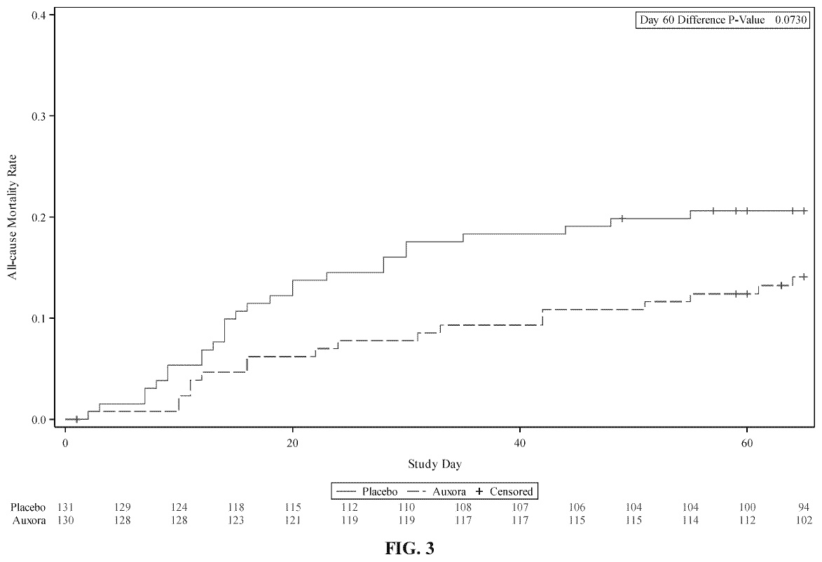

FIG. 4 is a chart showing an example of a clinical trial design for evaluating CRAC inhibitors in ventilated patients.

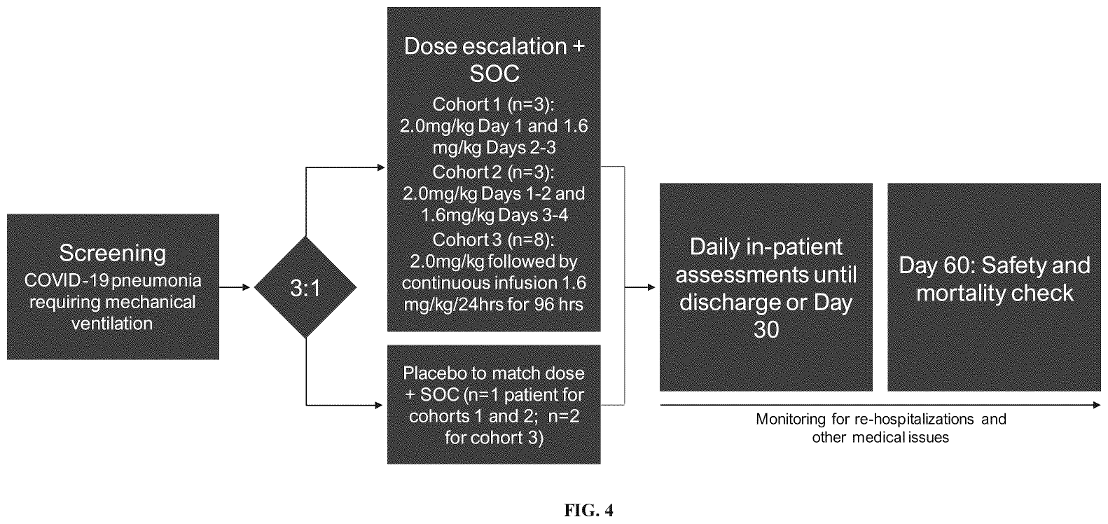

FIG. 5 is a chart showing clinical trial for patients receiving Auxora when the patients have different PaO₂/FiO₂ ranges.

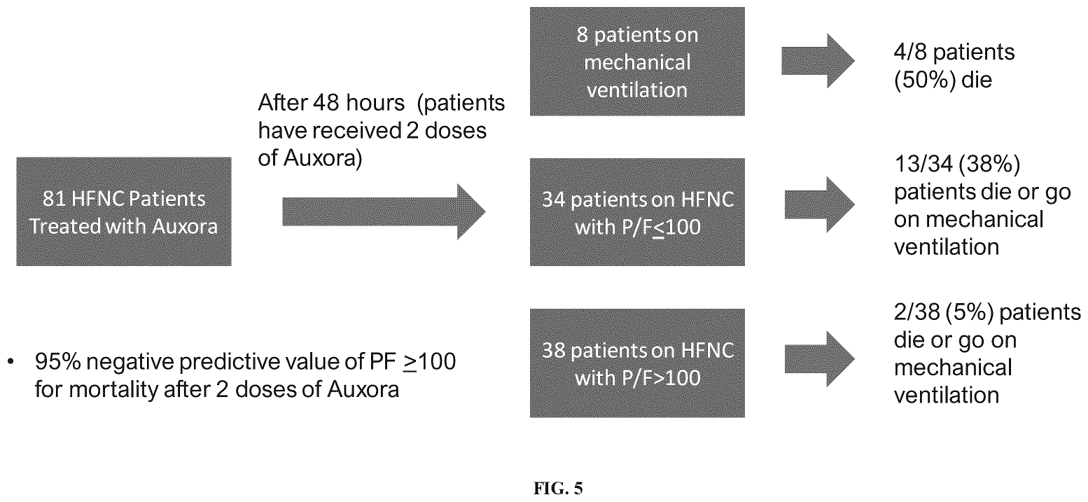

FIG. 6 is a chart showing an example of a clinical trial design for evaluating combination therapy with CRAC inhibitors and tocilizumab in patients with severe COVID-19 pneumonia.

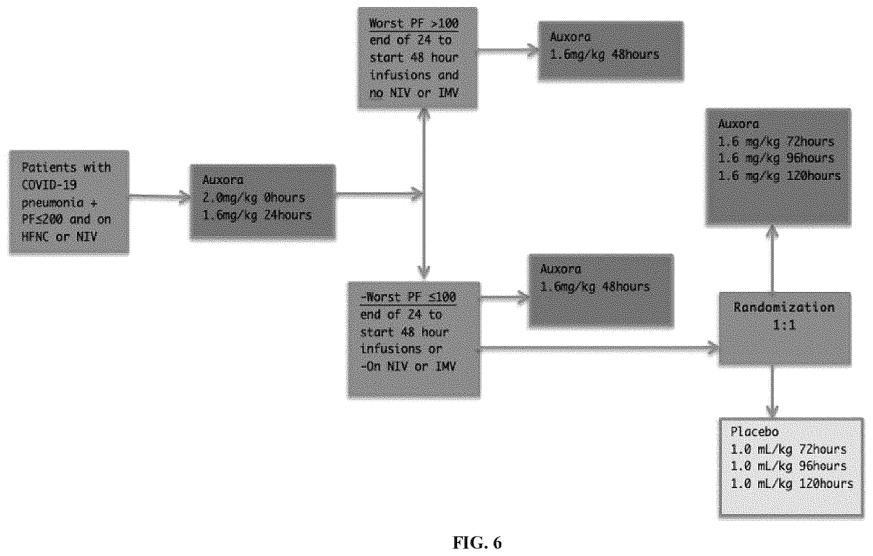

FIG. 7 is a chart showing imputed PaO₂/FiO₂ data.

FIG. 8 shows the schedule for the clinical trials.

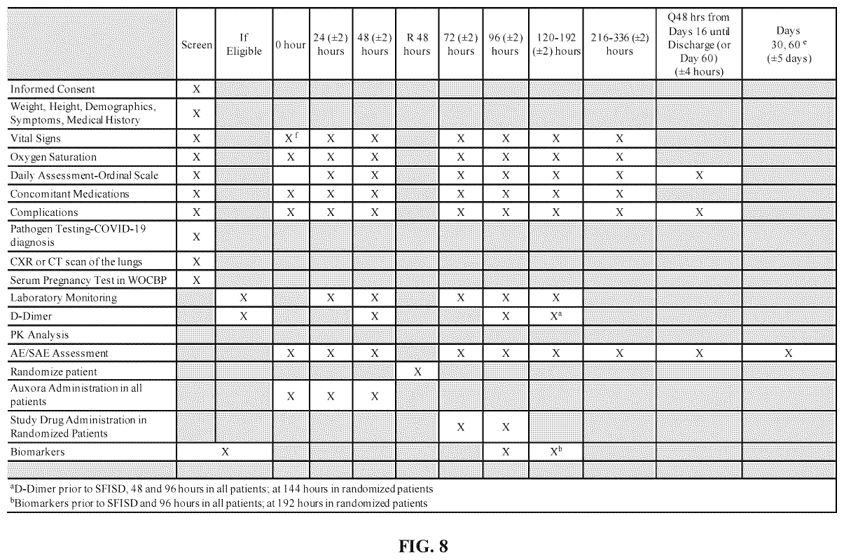

### DETAILED DESCRIPTION OF THE INVENTION

Methods and compositions disclosed herein are used for modulating intracellular calcium to treat or prevent acute lung injury (ALI) and/or acute respiratory distress syndrome (ARDS). In some aspects, methods and compounds provided herein modulate CRAC channel activity. In another aspect, methods and compounds provided herein reduce the number of functional CRAC channels. In some aspects, methods and compounds described herein are CRAC channel blockers or CRAC channel modulators. In some aspects, methods and compounds described herein modulate the PaO₂/FiO₂ (P/F) ratio of the subject. In some aspects, methods and compounds described herein can further include one or more additional therapeutic agents such as a corticosteroid and/or immunosuppressive drug.

Calcium plays a vital role in cell function and survival. Specifically, calcium is a key element in the transduction of signals into and within cells. Cellular responses to growth factors, neurotransmitters, hormones and a variety of other signal molecules are initiated through calcium-dependent processes.

Almost all cell types depend in some manner upon the generation of cytoplasmic Ca²⁺ signals to regulate cell function, or to trigger specific responses. Cytosolic Ca²⁺ signals control a wide array of cellular functions ranging from short-term responses such as contraction and secretion to longer-term regulation of cell growth and proliferation. Usually, these signals involve some combination of release of Ca²⁺ from intracellular stores, such as the endoplasmic reticulum (ER), and influx of Ca²⁺ across the plasma membrane. In one example, cell activation begins with an agonist binding to a surface membrane receptor, which is coupled to phospholipase C (PLC) through a G-protein mechanism. PLC activation leads to the production of inositol 1,4,5-triphosphate (IP₃), which in turn activates the IP₃ receptor causing release of Ca²⁺ from the ER. The fall in ER Ca²⁺ then signals to activate plasma membrane store-operated calcium (SOC) channels.

Store-operated calcium (SOC) influx is a process in cellular physiology that controls such diverse functions such as, but not limited to, refilling of intracellular Ca²⁺ stores (Putney et al. Cell, 75, 199-201, 1993), activation of enzymatic activity (Fagan et al., J. Biol. Chem. 275:26530-26537, 2000), gene transcription (Lewis, Annu. Rev. Immunol. 19:497-521, 2001), cell proliferation (Nunez et al., J. Physiol. 571.1, 57-73, 2006), and release of cytokines (Winslow et al., Curr. Opin. Immunol. 15:299-307, 2003). In some nonexcitable cells, e.g., blood cells, immune cells, hematopoietic cells, T lymphocytes and mast cells, pancreatic acinar cells (PACs), epithelial and ductal cells of other glands (e.g. salivary glands), endothelial and endothelial progenitor cells (e.g., pulmonary endothelial cells), SOC influx occurs through calcium release-activated calcium (CRAC) channels, a type of SOC channel.

The calcium influx mechanism has been referred to as store-operated calcium entry (SOCE). Stromal interaction molecule (STIM) proteins are an essential component of SOC channel function, serving as the sensors for detecting the depletion of calcium from intracellular stores and for activating SOC channels.

Preclinical and animal work provided herein show that CRAC inhibitors such as N-(5-(6-Chloro-2,2-difluorobenzo[d][1,3]dioxol-5-yl)pyrazin-2-yl)-2-fluoro-6-methylbenzamide (Compound 1) can treat COVID-19 pneumonia, other viral pneumonias, and ALI or ARDS. Compound 1 has been tested in acutely ill acute pancreatitis (AP) patients with systemic inflammatory response syndrome (SIRS) and hypoxemia (and patients with severe and/or critical COVID-19 pneumonia). Treatment of a subject with CRAC inhibitors may lead to a rapid reduction of IL-6, IL-2, IL-17, TNFα, and/or other inflammatory cytokines in the subject. Treatment of a subject with CRAC inhibitors such as Compound 1 may lead to preservation of lung function and protection from injury.

### Calcium Homeostasis

Cellular calcium homeostasis is a result of the summation of regulatory systems involved in the control of intracellular calcium levels and movements. Cellular calcium homeostasis is achieved, at least in part, by calcium binding and by movement of calcium into and out of the cell across the plasma membrane and within the cell by movement of calcium across membranes of intracellular organelles including, for example, the endoplasmic reticulum, sarcoplasmic reticulum, mitochondria and endocytic organelles including endosomes and lysosomes.

Movement of calcium across cellular membranes is carried out by specialized proteins. For example, calcium from the extracellular space can enter the cell through various calcium channels and a sodium/calcium exchanger and is actively extruded from the cell by calcium pumps and sodium/calcium exchangers. Calcium can also be released from internal stores through inositol trisphosphate or ryanodine receptors and can be taken up by these organelles by means of calcium pumps.

Calcium can enter cells by any of several general classes of channels, including but not limited to, voltage-operated calcium (VOC) channels, ligand-gated calcium channels, store-operated calcium (SOC) channels, and sodium/calcium exchangers operating in reverse mode. VOC channels are activated by membrane depolarization and are found in excitable cells like nerve and muscle and are for the most part not found in nonexcitable cells. Under some conditions, Ca²⁺ can enter cells via Na+-Ca²⁺ exchangers operating in reverse mode.

Endocytosis provides another process by which cells can take up calcium from the extracellular medium through endosomes. In addition, some cells, e.g., exocrine cells, can release calcium via exocytosis.

Cytosolic calcium concentration is tightly regulated with resting levels usually estimated at approximately 0.1 μM in mammalian cells, whereas the extracellular calcium concentration is typically about 2 mM. This tight regulation facilitates transduction of signals into and within cells through transient calcium flux across the plasma membrane and membranes of intracellular organelles. There is a multiplicity of intracellular calcium transport and buffer systems in cells that serve to shape intracellular calcium signals and maintain the low resting cytoplasmic calcium concentration. In cells at rest, the principal components involved in maintaining basal calcium levels are calcium pumps and leak pathways in both the endoplasmic reticulum and plasma membrane. Disturbance of resting cytosolic calcium levels can affect transmission of calcium-dependent signals and give rise to defects in a number of cellular processes. For example, cell proliferation involves a prolonged calcium signaling sequence. Other cellular processes that involve calcium signaling include, but are not limited to, secretion, transcription factor signaling, and fertilization.

Cell-surface receptors that activate phospholipase C (PLC) create cytosolic Ca²⁺ signals from intra- and extra-cellular sources. An initial transient rise of [Ca²⁺]i (intracellular calcium concentration) results from the release of Ca²⁺ from the endoplasmic reticulum (ER), which is triggered by the PLC product, inositol-1,4,5-trisphosphate (IP₃), opening IP₃ receptors in the ER (Streb et al. Nature, 306, 67-69, 1983). A subsequent phase of sustained Ca²⁺ entry across the plasma membrane then ensues, through specialized store operated calcium (SOC) channels (in the case of non-excitable cells like immune PAC cells, the SOC channels are calcium release-activated calcium (CRAC) channels) in the plasma membrane. Store-operated Ca²⁺ entry (SOCE) is the process in which the emptying of Ca²⁺ stores itself activates Ca²⁺ channels in the plasma membrane to help refill the stores (Putney, Cell Calcium, 7, 1-12, 1986; Parekh et al., Physiol. Rev. 757-810; 2005). SOCE does more than simply provide Ca²⁺ for refilling stores, but can itself generate sustained Ca²⁺ signals that control such essential functions as gene expression, cell metabolism and exocytosis (Parekh and Putney, Physiol. Rev. 85, 757-810 (2005).

In lymphocytes and mast cells, activation of antigen or Fc receptors, respectively causes the release of Ca²⁺ from intracellular stores, which in turn leads to Ca²⁺ influx through CRAC channels in the plasma membrane. In some immune cells, including monocyte/macrophage, neutrophils, or dendritic cells, release of Ca²⁺ from intracellular stores and Ca²⁺ influx through CRAC channels in the plasma membrane may result without Fc receptor activation. The subsequent rise in intracellular Ca²⁺ activates calcineurin, a phosphatase that regulates the transcription factor NFAT. In resting cells, NFAT is phosphorylated and resides in the cytoplasm, but when dephosphorylated by calcineurin, NFAT translocates to the nucleus and activates different genetic programs depending on stimulation conditions and cell type. In response to infections and during transplant rejection, NFAT partners with the transcription factor AP-1 (Fos-Jun) in the nucleus of “effector” T cells, thereby trans-activating cytokine genes, genes that regulate T cell proliferation and other genes that orchestrate an active immune response (Rao et al., Annu Rev Immunol., 1997; 15:707-47). In contrast, in T cells recognizing self-antigens, NFAT is activated in the absence of AP-1, and activates a transcriptional program known as “anergy” that suppresses autoimmune responses (Macian et al., Transcriptional mechanisms underlying lymphocyte tolerance. Cell. 2002 Jun. 14; 109 (6): 719-31). In a subclass of T cells known as regulatory T cells which suppress autoimmunity mediated by self-reactive effector T cells, NFAT partners with the transcription factor FOXP3 to activate genes responsible for suppressor function (Wu et al., Cell, 2006 Jul. 28; 126 (2): 375-87; Rudensky AY, Gavin M, Zheng Y. Cell. 2006 Jul. 28; 126 (2): 253-256).

The endoplasmic reticulum (ER) carries out a variety processes. The ER has a role as both a Ca²⁺ sink and an agonist-sensitive Ca²⁺ store, and protein folding/processing takes place within its lumen. In the latter case, numerous Ca²⁺-dependent chaperone proteins ensure that newly synthesized proteins are folded correctly and sent off to their appropriate destination. The ER is also involved in vesicle trafficking, release of stress signals, regulation of cholesterol metabolism, and apoptosis. Many of these processes require intraluminal Ca²⁺ and protein misfolding, ER stress responses, and apoptosis can all be induced by depleting the ER of Ca²⁺ for prolonged periods of time. Because it contains a finite amount of Ca²⁺, it is clear that ER Ca²⁺ content must fall after release of that Ca²⁺ during stimulation. However, to preserve the functional integrity of the ER, it is vital that the Ca²⁺ content does not fall too low or is maintained at least at a low level. Replenishment of the ER with Ca²⁺ is therefore a central process to all eukaryotic cells. Because a fall in ER Ca²⁺ content activates store-operated Ca²⁺ channels in the plasma membrane, a major function of this Ca²⁺ entry pathway is believed to be maintenance of ER Ca²⁺ levels that are necessary for proper protein synthesis and folding. However, store-operated Ca²⁺ channels have other important roles.

The understanding of store-operated calcium entry was provided by electrophysiological studies which established that the process of emptying the stores activated a Ca²⁺ current in mast cells called Ca²⁺ release-activated Ca²⁺ current or ICRAC. ICRAC is non-voltage activated, inwardly rectifying, and remarkably selective for Ca²⁺. It is found in several cell types mainly of hematopoietic origin. ICRAC is not the only store-operated current, and it is now apparent that store-operated influx encompasses a family of Ca²⁺-permeable channels, with different properties in different cell types. ICRAC was the first store-operated Ca²⁺ current to be described and remains a popular model for studying store-operated influx.

Store-operated calcium channels can be activated by any procedure that empties ER Ca²⁺ stores; it does not seem to matter how the stores are emptied, the net effect is activation of store-operated Ca²⁺ entry. Physiologically, store emptying is evoked by an increase in the levels of IP₃ or other Ca²⁺-releasing signals followed by Ca²⁺ release from the stores. But there are several other methods for emptying stores. These methods include the following:
1) elevation of IP₃ in the cytosol (following receptor stimulation or, dialyzing the cytosol with IP₃ itself or related congeners like the nonmetabolizable analog Ins (2,4,5)P₃);
2) application of a Ca²⁺ ionophore (e.g., ionomycin) to permeabilize the ER membrane;
3) dialyzing the cytoplasm with high concentrations of Ca²⁺ chelators (e.g., EGTA or BAPTA), which chelate Ca²⁺ that leaks from the stores and hence prevent store refilling;
4) exposure to the sarcoplasmic/endoplasmic reticulum Ca²⁺-ATPase (SERCA) inhibitors like thapsigargin, cyclopiazonic acid, and di-tert-butylhydroquinone;
5) sensitizing the IP₃ receptors to resting levels of InsP3 with agents like thimerosal; and
6) loading membrane-permeable metal Ca²⁺ chelators like N,N,N′,N′-tetrakis(2-pyridylmethyl)ethylene diamine (TPEN) directly into the stores.
   Through mass action, TPEN lowers free intraluminal Ca²⁺ concentration without changing total store Ca²⁺ such that the store depletion-dependent signal is generated.

These methods of emptying stores are not devoid of potential problems. The key feature of store-operated Ca²⁺ entry is that it is the fall in Ca²⁺ content within the stores and not the subsequent rise in cytoplasmic Ca²⁺ concentration that activates the channels. However, ionomycin and SERCA pump blockers generally cause a rise in cytoplasmic Ca²⁺ concentration as a consequence of store depletion, and such a rise in Ca²⁺ could open Ca²⁺-activated cation channels permeable to Ca²⁺. One way to avoid such problems is to use agents under conditions where cytoplasmic Ca²⁺ has been strongly buffered with high concentrations of Ca²⁺ chelator such as EGTA or BAPTA.

### Store-Operated Calcium Entry

Reduced calcium concentration in intracellular calcium stores such as the endoplasmic reticulum resulting from release of calcium therefrom provides a signal for influx of calcium from the extracellular medium into the cell. This influx of calcium, which produces a sustained “plateau” elevation of cytosolic calcium concentration, generally does not rely on voltage-gated plasma membrane channels and does not involve activation of calcium channels by calcium. This calcium influx mechanism is referred to as capacitive calcium entry (CCE), calcium release-activated, store-operated or depletion-operated calcium entry. Store-operated calcium entry can be recorded as an ionic current with distinctive properties. This current is referred to as I_{SOC} (store-operated current) or ICRAC (calcium release-activated current).

Electrophysiological analysis of store-operated or calcium release-activated currents reveal distinct biophysical properties (see, e.g., Parekh and Penner (1997) Physiol. Rev. 77:901-930) of these currents. For example, the current can be activated by depletion of intracellular calcium stores (e.g., by non-physiological activators such as thapsigargin, CPA, ionomycin and BAPTA, and physiological activators such as IP₃) and can be selective for divalent cations, such as calcium, over monovalent ions in physiological solutions or conditions, can be influenced by changes in cytosolic calcium levels, and can show altered selectivity and conductivity in the presence of low extracellular concentrations of divalent cations. The current may also be blocked or enhanced by 2-APB (depending on concentration) and blocked by SKF96365 and Gd³⁺ and generally can be described as a calcium current that is not strictly voltage-gated.

Patch-clamp studies in mast cells and Jurkat leukemic T cells have established the CRAC entry mechanism as an ion channel with distinctive biophysical characteristics, including a high selectivity for Ca²⁺ paired with an exceedingly low conductance. Furthermore, the CRAC channel was shown to fulfill the rigorous criteria for being store-operated, which is the activation solely by the reduction of Ca²⁺ in the ER rather than by cytosolic Ca²⁺ or other messengers generated by PLC (Prakriya et al., In Molecular and Cellular Insights into Ion Channel Biology (ed. Robert Maue) 121-140 (Elsevier Science, Amsterdam, 2004)).

### Regulation of Store-Operated Calcium Entry by Intracellular Calcium Stores

Store-operated calcium entry is regulated by the level of calcium within an intracellular calcium store. Intracellular calcium stores can be characterized by sensitivity to agents, which can be physiological or pharmacological, which activate release of calcium from the stores or inhibit uptake of calcium into the stores. Different cells have been studied in characterization of intracellular calcium stores, and stores have been characterized as sensitive to various agents, including, but not limited to, IP₃ and compounds that effect the IP₃ receptor, thapsigargin, ionomycin and/or cyclic ADP-ribose (cADPR) (see, e.g., Berridge (1993) Nature 361:315-325; Churchill and Louis (1999) Am. J. Physiol. 276: C₄26-C₄34; Dargie et al. (1990) Cell Regul. 1:279-290; Gerasimenko et al. (1996) Cell 84:473-480; Gromoda et al. (1995) FEBS Lett. 360:303-306; Guse et al. (1999) Nature 398:70-73).

Accumulation of calcium within endoplasmic reticulum and sarcoplasmic reticulum (SR; a specialized version of the endoplasmic reticulum in striated muscle) storage organelles is achieved through sarcoplasmic-endoplasmic reticulum calcium ATPases (SERCAs), commonly referred to as calcium pumps. During signaling (i.e., when endoplasmic reticulum channels are activated to provide for calcium release from the endoplasmic reticulum into the cytoplasm), endoplasmic reticulum calcium is replenished by the SERCA pump with cytoplasmic calcium that has entered the cell from the extracellular medium (Yu and Hinkle (2000) J. Biol. Chem. 275:23648-23653; Hofer et al. (1998) EMBO J. 17:1986-1995).

Calcium release channels associated with IP₃ and ryanodine receptors provide for controlled release of calcium from endoplasmic and sarcoplasmic reticulum into the cytoplasm resulting in transient increases in cytoplasmic calcium concentration. IP₃ receptor-mediated calcium release is triggered by IP₃ formed by the breakdown of plasma membrane phosphoinositides through the action of phospholipase C, which is activated by binding of an agonist to a plasma membrane G protein-coupled receptor or tyrosine kinase. Ryanodine receptor-mediated calcium release is triggered by an increase in cytoplasmic calcium and is referred to as calcium-induced calcium release (CICR). The activity of ryanodine receptors (which have affinity for ryanodine and caffeine) may also be regulated by cyclic ADP-ribose.

Thus, the calcium levels in the stores, and in the cytoplasm, fluctuate. For example, ER free calcium concentration can decrease from a range of about 60-400 μM to about 1-50 μM when HeLa cells are treated with histamine, an agonist of PLC-linked histamine receptors (Miyawaki et al. (1997) Nature 388:882-887). Store-operated calcium entry is activated as the free calcium concentration of the intracellular stores is reduced. Depletion of store calcium, as well as a concomitant increase in cytosolic calcium concentration, can thus regulate store-operated calcium entry into cells.

### Cytoplasmic Calcium Buffering

Agonist activation of signaling processes in cells can involve dramatic increases in the calcium permeability of the endoplasmic reticulum, for example, through opening of IP₃ receptor channels, and the plasma membrane through store-operated calcium entry. These increases in calcium permeability are associated with an increase in cytosolic calcium concentration that can be separated into two components: a “spike” of calcium release from the endoplasmic reticulum during activation of the IP₃ receptor and a plateau phase which is a sustained elevation of calcium levels resulting from entry of calcium into the cytoplasm from the extracellular medium. Upon stimulation, the resting intracellular free calcium concentration of about 100 nM can rise globally to greater than 1 μM and higher in microdomains of the cell. The cell modulates these calcium signals with endogenous calcium buffers, including physiological buffering by organelles such as mitochondria, endoplasmic reticulum and Golgi. Mitochondrial uptake of calcium through a uniporter in the inner membrane is driven by the large negative mitochondrial membrane potential, and the accumulated calcium is released slowly through sodium-dependent and -independent exchangers, and, under some circumstances, the permeability transition pore (PTP). Thus, mitochondria can act as calcium buffers by taking up calcium during periods of cellular activation and can slowly release it later. Uptake of calcium into the endoplasmic reticulum is regulated by the sarcoplasmic and endoplasmic reticulum calcium ATPase (SERCA). Uptake of calcium into the Golgi is mediated by a P-type calcium transport ATPase (PMR₁/ATP2C₁). Additionally, there is evidence that a significant amount of the calcium released upon IP₃ receptor activation is extruded from the cell through the action of the plasma membrane calcium ATPase. For example, plasma membrane calcium ATPases provide the dominant mechanism for calcium clearance in human T cells and Jurkat cells, although sodium/calcium exchange also contributes to calcium clearance in human T cells. Within calcium-storing organelles, calcium ions can be bound to specialized calcium-buffering proteins, such as, for example, calsequestrins, calreticulins and calnexins. Additionally, there are calcium-buffering proteins in the cytosol that modulate calcium spikes and assist in redistribution of calciumions. Thus, proteins and other molecules that participate in any of these and other mechanisms through which cytosolic calcium levels can be reduced are proteins that are involved in, participate in and/or provide for cytoplasmic calcium buffering. Thus, cytoplasmic calcium buffering helps regulate cytoplasmic Ca²⁺ levels during periods of sustained calcium influx through SOC channels or bursts of Ca²⁺ release. Large increases in cytoplasmic Ca²⁺ levels or store refilling deactivate SOCE.

### Downstream Calcium Entry-Mediated Events

In addition to intracellular changes in calcium stores, store-operated calcium entry affects a multitude of events that are consequent to or in addition to the store-operated changes. For example Ca²⁺ influx results in the activation of a large number of calmodulin-dependent enzymes including the serine phosphatase calcineurin. Activation of calcineurin by an increase in intracellular calcium results in acute secretory processes such as mast cell degranulation. Activated mast cells release preformed granules containing histamine, heparin, TNFα and enzymes such as β-hexosaminidase. Some cellular events, such as B and T cell proliferation, require sustained calcineurin signaling, which requires a sustained increase in intracellular calcium. A number of transcription factors are regulated by calcineurin, including NFAT (nuclear factor of activated T cells), MEF₂ and NFκB. NFAT transcription factors play important roles in many cell types, including immune cells. In immune cells NFAT mediates transcription of a large number of molecules, including cytokines, chemokines and cell surface receptors. Transcriptional elements for NFAT have been found within the promoters of cytokines such as IL-2, IL-3, IL-4, IL-5, IL-8, IL-13, as well as tumor necrosis factor alpha (TNFα), granulocyte colony-stimulating factor (G-CSF), and gamma-interferon (γ-IFN).

The activity of NFAT proteins is regulated by their phosphorylation level, which in turn is regulated by both calcineurin and NFAT kinases. Activation of calcineurin by an increase in intracellular calcium levels results in dephosphorylation of NFAT and entry into the nucleus. Rephosphorylation of NFAT masks the nuclear localization sequence of NFAT and prevents its entry into the nucleus. Because of its strong dependence on calcineurin-mediated dephosphorylation for localization and activity, NFAT is a sensitive indicator of intracellular free calcium levels.

### CRAC Channels and Immune Responses

CRAC channels are located in the plasma membrane and open in response to the release of Ca²⁺ from endoplasmic reticulum stores. In immune cells, stimulation of cell surface receptors activates CRAC channels, leading to Ca²⁺ entry and cytokine production. Cells of both the adaptive and innate immune system (e.g., T-cells, neutrophils and macrophages) are known to be regulated by CRAC channels. CRAC channels also play a role in the activation of endothelial cells, which are involved in the pathogenesis of ALI/ARDS. The normal pulmonary endothelium maintains a tight barrier between endothelial cells, the pulmonary interstitium, and the alveolar space, thereby enabling gas exchange. In inflammatory conditions, stimulation of receptors on pulmonary endothelial cells leads to activation of Ca²⁺ entry via CRAC channels. The Ca²⁺ entry leads to loss of barrier function, which in turn causes leakage of protein-rich fluid into the alveolus, impaired gas exchange, and hypoxemia.

Stimulation of T cell receptors causes depletion of intracellular Ca²⁺ stores and subsequent opening of the CRAC (Ca²⁺-release-activated Ca²⁺) channels. A sustained increase in intracellular Ca²⁺ concentration activates the calcineurin/NFAT (nuclear factor of activated T cells) pathway and turns on transcriptional programs of various cytokines. Orai1 and STIM1 are identified as a long-sought pore component of CRAC channels and as an endoplasmic reticulum (ER) Ca²⁺ sensor, respectively. STIM1 senses Ca²⁺ depletion in ER after stimulation of T cell receptors, translocates to plasma membrane (PM) proximal ER, binds to and activates Orai1. Human patients deficient in Orai1 or STIM1 have severe combined immune deficiency.

### Calcium Channel Inhibitors

Disclosed herein are a number of Calcium channel inhibitors consistent with the methods, compositions, administration regimens and compositions for use disclosed herein. In some embodiments, a Calcium channel inhibitor comprises a SOC inhibitor. In some embodiments, a Calcium channel inhibitor is a SOC inhibitor. In some embodiments, the SOC comprises a CRAC. In some embodiments the SOC inhibitor comprises a CRAC inhibitor. In some embodiments the Calcium channel inhibitor is a CRAC inhibitor. In some embodiments, the Calcium channel inhibitor inhibits a channel comprising STIM1 protein. In some embodiments, the CRAC comprises an Orai1 protein. In some embodiments, the CRAC inhibitor comprises an Orai1 protein inhibitor. In some embodiments, the Calcium channel inhibitor inhibits a channel comprising Orai1 protein. In some embodiments, the CRAC comprises an Orai2 protein. In some embodiments, the CRAC inhibitor comprises an Orai2 protein inhibitor. In some embodiments, the Calcium channel inhibitor inhibits a channel comprising Orai2 protein.

In some embodiments the compound is a compound having the structure of:

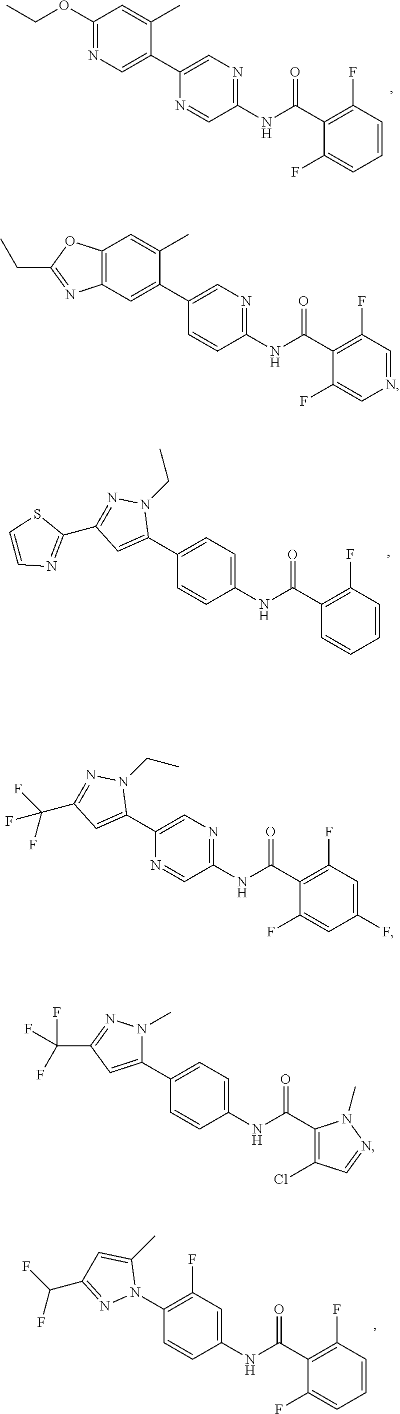 [SMILES: CCOc1cc(C)c(-c2cnc(NC(=O)c3c(F)cccc3F)cn2)cn1 ; CCc1nc2cc(-c3ccc(NC(=O)c4c(F)cncc4F)nc3)c(C)cc2o1 ; CCn1nc(-c2nccs2)cc1-c1ccc(NC(=O)c2ccccc2F)cc1 ; CCn1nc(C(F)(F)F)cc1-c1cnc(NC(=O)c2c(F)cc(F)cc2F)cn1 ; Cn1nc(C(F)(F)F)cc1-c1ccc(NC(=O)c2c(Cl)cnn2C)cc1 ; Cc1cc(C(F)F)nn1-c1ccc(NC(=O)c2c(F)cccc2F)cc1F]

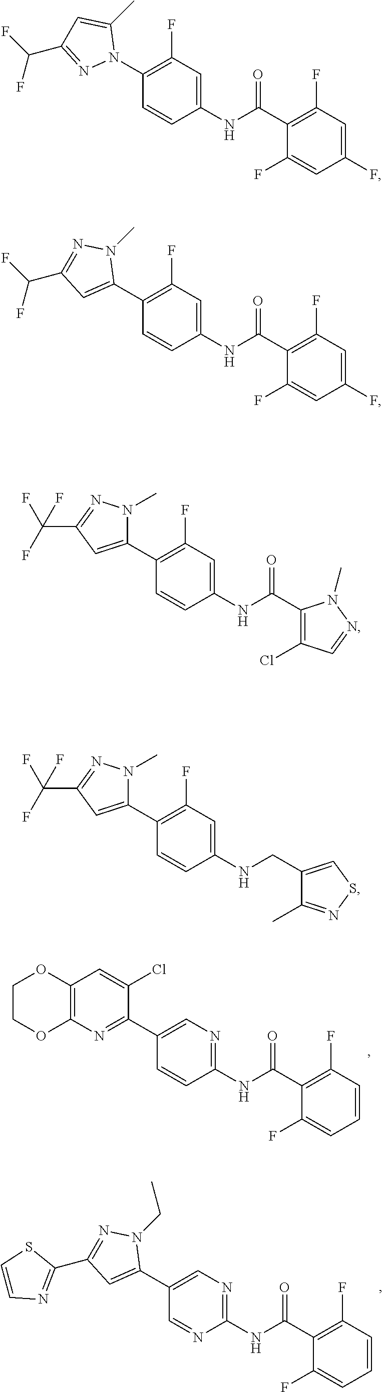 [SMILES: Cc1cc(C(F)F)nn1-c1ccc(NC(=O)c2c(F)cc(F)cc2F)cc1F ; Cn1nc(C(F)F)cc1-c1ccc(NC(=O)c2c(F)cc(F)cc2F)cc1F ; Cn1nc(C(F)(F)F)cc1-c1ccc(NC(=O)c2c(Cl)cnn2C)cc1F ; Cc1nscc1CNc1ccc(-c2cc(C(F)(F)F)nn2C)c(F)c1 ; O=C(Nc1ccc(-c2nc3c(cc2Cl)OCCO3)cn1)c1c(F)cccc1F ; CCn1nc(-c2nccs2)cc1-c1cnc(NC(=O)c2c(F)cccc2F)nc1]

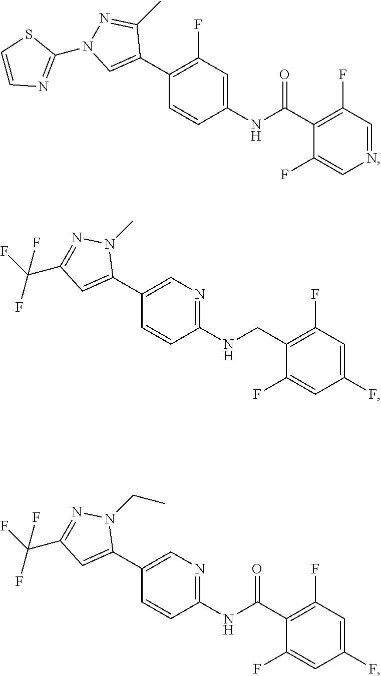 [SMILES: Cc1nn(-c2nccs2)cc1-c1ccc(NC(=O)c2c(F)cncc2F)cc1F ; Cn1nc(C(F)(F)F)cc1-c1ccc(NCc2c(F)cc(F)cc2F)nc1 ; CCn1nc(C(F)(F)F)cc1-c1ccc(NC(=O)c2c(F)cc(F)cc2F)nc1]

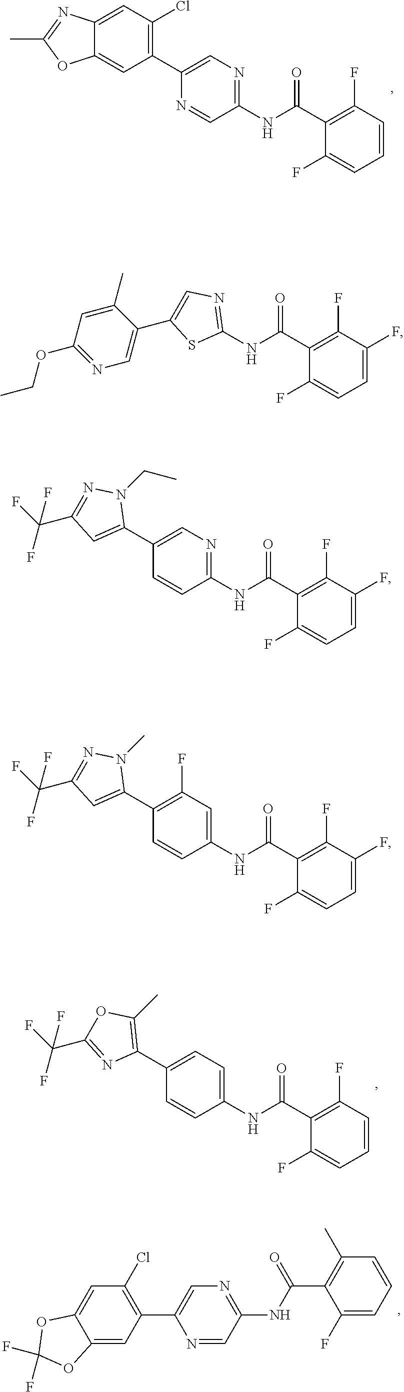 [SMILES: Cc1nc2cc(Cl)c(-c3cnc(NC(=O)c4c(F)cccc4F)cn3)cc2o1 ; CCOc1cc(C)c(-c2cnc(NC(=O)c3c(F)ccc(F)c3F)s2)cn1 ; CCn1nc(C(F)(F)F)cc1-c1ccc(NC(=O)c2c(F)ccc(F)c2F)nc1 ; Cn1nc(C(F)(F)F)cc1-c1ccc(NC(=O)c2c(F)ccc(F)c2F)cc1F ; Cc1oc(C(F)(F)F)nc1-c1ccc(NC(=O)c2c(F)cccc2F)cc1 ; Cc1cccc(F)c1C(=O)Nc1cnc(-c2cc3c(cc2Cl)OC(F)(F)O3)cn1]

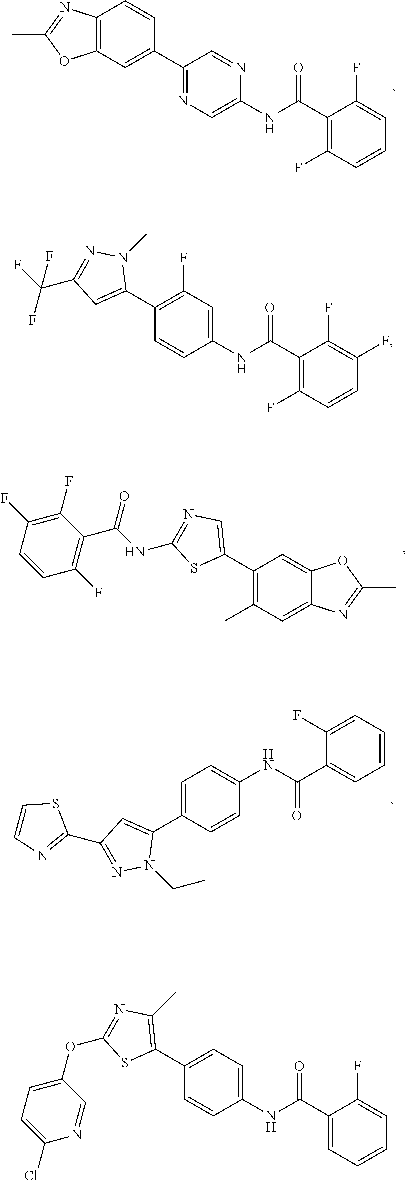 [SMILES: Cc1nc2ccc(-c3cnc(NC(=O)c4c(F)cccc4F)cn3)cc2o1 ; Cn1nc(C(F)(F)F)cc1-c1ccc(NC(=O)c2c(F)ccc(F)c2F)cc1F ; Cc1nc2cc(C)c(-c3cnc(NC(=O)c4c(F)ccc(F)c4F)s3)cc2o1 ; CCn1nc(-c2nccs2)cc1-c1ccc(NC(=O)c2ccccc2F)cc1 ; Cc1nc(Oc2ccc(Cl)nc2)sc1-c1ccc(NC(=O)c2ccccc2F)cc1]

or a pharmaceutically acceptable salt, pharmaceutically acceptable solvate, or pharmaceutically acceptable prodrug thereof. In some embodiments the compound is selected form a list of compounds consisting: N-(5-(6-chloro-2,2-difluorobenzo[d][1,3]dioxol-5-yl)pyrazin-2-yl)-2-fluoro-6-methylbenzamide. In some aspects the intracellular Calcium signaling inhibitor is a compound of N-(5-(6-chloro-2,2-difluorobenzo[d][1,3]dioxol-5-yl)pyrazin-2-yl)-2-fluoro-6-methylbenzamide or a pharmaceutically acceptable salt, pharmaceutically acceptable solvate, or pharmaceutically acceptable prodrug thereof.

In some embodiments, the intracellular Calcium signaling inhibitor is a CRACT inhibitor. In some aspects the intracellular Calcium signaling inhibitor is chosen from among the compounds, N-(5-(6-ethoxy-4-methylpyridin-3-yl)pyrazin-2-yl)-2,6-difluorobenzamide, N-(5-(2-ethyl-6-methylbenzo[d]oxazol-5-yl) pyridin-2-yl)-3,5-difluoroisonicotinamide, N-(4-(1-ethyl-3-(thiazol-2-yl)-1H-pyrazol-5-yl)phenyl)-2-fluorobenzamide, N-(5-(1-ethyl-3-(triflouromethyl)-1H-pyrazol-5-yl)pyrazin-2-yl)-2,4,6-trifluorobenzamide, 4-chloro-1-methyl-N-(4-(1-methyl-3-(trifluoromethyl)-1H-pyrazol-5-yl)phenyl)-1H-pyrazole-5-carboxamide, N-(4-(3-(difluoromethyl)-5-methyl-1H-pyrazol-1-yl)-3-fluorophenyl)-2,6-difluorobenzamide, N-(4-(3-(difluoromethyl)-5-methyl-1H-pyrazol-1-yl)-3-fluorophenyl)-2,4,6-trifluorobenzamide, N-(4-(3-(difluoromethyl)-1-methyl-1H-pyrazol-5-yl)-3-fluorophenyl)-2,4,6-trifluorobenzamide, 4-chloro-N-(3-fluoro-4-(1-methyl-3-(trifluoromethyl)-1H-pyrazol-5-yl)phenyl)-1-methyl-1H-pyrazole-5-carboxamide, 3-fluoro-4-(1-methyl-3-(trifluoromethyl)-1H-pyrazol-5-yl)-N-((3-methylisothiazol-4-yl)methyl) aniline, N-(5-(7-chloro-2,3-dihydro-[1,4]dioxino[2,3-b]pyridin-6-yl) pyridin-2-yl)-2,6-difluorobenzamide, N-(2,6-difluorobenzyl)-5-(1-ethyl-3-(thiazol-2-yl)-1H-pyrazol-5-yl)pyrimidin-2-amine, 3,5-difluoro-N-(3-fluoro-4-(3-methyl-1-(thiazol-2-yl)-1H-pyrazol-4-yl)phenyl) isonicotinamide, 5-(1-methyl-3-(trifluoromethyl)-1H-pyrazol-5-yl)-N-(2,4,6-trifluorobenzyl)pyridin-2-amine, N-(5-(1-ethyl-3-(trifluoromethyl)-1H-pyrazol-5-yl) pyridin-2-yl)-2,4,6-trifluorobenzamide, N-(5-(5-chloro-2-methylbenzo[d]oxazol-6-yl)pyrazin-2-yl)-2,6-difluorobenzamide, N-(5-(6-ethoxy-4-methylpyridin-3-yl) thiazol-2-yl)-2,3,6-trifluorobenzamide, N-(5-(1-ethyl-3-(trifluoromethyl)-1H-pyrazol-5-yl) pyridin-2-yl)-2,3,6-trifluorobenzamide, 2,3,6-trifluoro-N-(3-fluoro-4-(1-methyl-3-(trifluoromethyl)-1H-pyrazol-5-yl)phenyl)benzamide, 2,6-difluoro-N-(4-(5-methyl-2-(trifluoromethyl) oxazol-4-yl)phenyl)benzamide, or N-(5-(6-chloro-2,2-difluorobenzo[d][1,3]dioxol-5-yl)pyrazin-2-yl)-2-fluoro-6-methylbenzamide, 2,6-difluoro-N-(5-(2-methylbenzo[d]oxazol-6-yl)pyrazin-2-yl)benzamide, 2,3,6-trifluoro-N-(3-fluoro-4-(1-methyl-3-(trifluoromethyl)-1H-pyrazol-5-yl)phenyl)benzamide, N-(5-(2,5-dimethylbenzo[d]oxazol-6-yl) thiazol-2-yl)-2,3,6-trifluorobenzamide, N-(4-(1-ethyl-3-(thiazol-2-yl)-1H-pyrazol-5-yl)phenyl)-2-fluorobenzamide, N-(4-(2-((6-chloropyridin-3-yl)oxy)-4-methylthiazol-5-yl)phenyl)-2-fluorobenzamide, or a pharmaceutically acceptable salt, pharmaceutically acceptable solvate, or pharmaceutically acceptable prodrug thereof. Each of these compounds is an example of a Calcium channel inhibitor, a SOC inhibitor, or a CRAC inhibitor.

In some embodiments, the intracellular Calcium signaling inhibitor is N-(5-(6-ethoxy-4-methylpyridin-3-yl)pyrazin-2-yl)-2,6-difluorobenzamide, N-(5-(2-ethyl-6-methylbenzo[d]oxazol-5-yl) pyridin-2-yl)-3,5-difluoroisonicotinamide, N-(4-(1-ethyl-3-(thiazol-2-yl)-1H-pyrazol-5-yl)phenyl)-2-fluorobenzamide, N-(5-(1-ethyl-3-(triflouromethyl)-1H-pyrazol-5-yl)pyrazin-2-yl)-2,4,6-trifluorobenzamide, N-(4-(3-(difluoromethyl)-5-methyl-1H-pyrazol-1-yl)-3-fluorophenyl)-2,6-difluorobenzamide, N-(4-(3-(difluoromethyl)-5-methyl-1H-pyrazol-1-yl)-3-fluorophenyl)-2,4,6-trifluorobenzamide, N-(4-(3-(difluoromethyl)-1-methyl-1H-pyrazol-5-yl)-3-fluorophenyl)-2,4,6-trifluorobenzamide, 3-fluoro-4-(1-methyl-3-(trifluoromethyl)-1H-pyrazol-5-yl)-N-((3-methylisothiazol-4-yl)methyl) aniline, N-(5-(7-chloro-2,3-dihydro-[1,4]dioxino[2,3-b]pyridin-6-yl) pyridin-2-yl)-2,6-difluorobenzamide, N-(2,6-difluorobenzyl)-5-(1-ethyl-3-(thiazol-2-yl)-1H-pyrazol-5-yl)pyrimidin-2-amine, 3,5-difluoro-N-(3-fluoro-4-(3-methyl-1-(thiazol-2-yl)-1H-pyrazol-4-yl)phenyl) isonicotinamide, N-(5-(1-ethyl-3-(trifluoromethyl)-1H-pyrazol-5-yl) pyridin-2-yl)-2,4,6-trifluorobenzamide, N-(5-(6-ethoxy-4-methylpyridin-3-yl) thiazol-2-yl)-2,6-difluorobenzamide, N-(5-(1-ethyl-3-(trifluoromethyl)-1H-pyrazol-5-yl) pyridin-2-yl)-2,3,6-trifluorobenzamide, 2,3,6-trifluoro-N-(3-fluoro-4-(1-methyl-3-(trifluoromethyl)-1H-pyrazol-5-yl)phenyl)benzamide, 2,6-difluoro-N-(4-(5-methyl-2-(trifluoromethyl) oxazol-4-yl)phenyl)benzamide, 2,6-difluoro-N-(5-(2-methylbenzo[d]oxazol-6-yl)pyrazin-2-yl)benzamide, N-(4-(1-ethyl-3-(thiazol-2-yl)-1H-pyrazol-5-yl)phenyl)-2-fluorobenzamide, N-(4-(2-((6-chloropyridin-3-yl)oxy)-4-methylthiazol-5-yl)phenyl)-2-fluorobenzamide, N-(5-(2,5-dimethylbenzo[d]oxazol-6-yl) thiazol-2-yl)-2,3,6-trifluorobenzamide, or N-(5-(6-chloro-2,2-difluorobenzo[d][1,3]dioxol-5-yl)pyrazin-2-yl)-2-fluoro-6-methylbenzamide, or a pharmaceutically acceptable salt, pharmaceutically acceptable solvate, or pharmaceutically acceptable prodrug thereof. In some embodiments, the intracellular Calcium signaling inhibitor is 2,6-difluoro-N-(4-(5-methyl-2-(trifluoromethyl) oxazol-4-yl)phenyl)benzamide, N-(5-(7-chloro-2,3-dihydro-[1,4]dioxino[2,3-b]pyridin-6-yl) pyridin-2-yl)-2,6-difluorobenzamide, N-(2,6-difluorobenzyl)-5-(1-ethyl-3-(thiazol-2-yl)-1H-pyrazol-5-yl)pyrimidin-2-amine, 3,5-difluoro-N-(3-fluoro-4-(3-methyl-1-(thiazol-2-yl)-1H-pyrazol-4-yl)phenyl) isonicotinamide, N-(5-(2-ethyl-6-methylbenzo[d]oxazol-5-yl) pyridin-2-yl)-3,5-difluoroisonicotinamide, or N-(5-(6-chloro-2,2-difluorobenzo[d][1,3]dioxol-5-yl)pyrazin-2-yl)-2-fluoro-6-methylbenzamide, or a pharmaceutically acceptable salt, pharmaceutically acceptable solvate, or pharmaceutically acceptable prodrug thereof. In some embodiments, the intracellular Calcium signaling inhibitor is N-(5-(6-Chloro-2,2-difluorobenzo[d][1,3]dioxol-5-yl)pyrazin-2-yl)-2-fluoro-6-methylbenzamide or a pharmaceutically acceptable salt, pharmaceutically acceptable solvate, or pharmaceutically acceptable prodrug thereof.

Disclosed herein, in some embodiments, are CRAC inhibitors. In some embodiments, the CRAC inhibitor comprises SK&F 96365. In some embodiments, the CRAC inhibitor comprises Econazole. In some embodiments, the CRAC inhibitor comprises L-651582. In some embodiments, the CRAC inhibitor comprises a carboxanilide compound. In some embodiments, the CRAC inhibitor comprises a biaryl carboxanilide compound. In some embodiments, the CRAC inhibitor comprises a heterocyclic carboxanilide compound. In some embodiments, the CRAC inhibitor comprises RP4010. In some embodiments, the CRAC inhibitor comprises Synta-66 (N-(2′,5′-dimethoxy[1,1′-biphenyl]-4-yl)-3-fluoro-4-pyridinecarboxamide). In some embodiments, the CRAC inhibitor comprises ML-9 (1-(5-chloronaphthalene-1-sulfonyl) homopiperazine). In some embodiments, the CRAC inhibitor comprises capsaicin (8-methyl-N-vanillyl-(trans)-6-nonenamide). In some embodiments, the CRAC inhibitor comprises NPPB (5-nitro-2-(3-phenylpropylamino)-benzoic acid). In some embodiments, the CRAC inhibitor comprises DES (diethylstilbestrol). In some embodiments, the CRAC inhibitor comprises BEL (bromenol lactone, or E-6-(bromoethylene)tetrahydro-3-(1-naphthyl)-2H-pyran-2-one). In some embodiments, the CRAC inhibitor comprises Carboxyamidotriazole (CAI). In some embodiments, the CRAC inhibitor comprises R02959 (2,6-difluoro-N-{5-[4-methyl-1-(5-methyl-thiazol-2-yl)-1,2,5,6-tetrahydro-pyridin-3-yl]-pyrazin-2-yl}-benzamide). In some embodiments, the CRAC inhibitor comprises a Tanshinone IIA sulfonate. In some embodiments, the CRAC inhibitor comprises sodium Tanshinone IIA sulfonate. In some embodiments, the CRAC inhibitor comprises MRS1845. Each of these compounds is also an example of a Calcium channel inhibitor, or of a SOC inhibitor.

Disclosed herein, in some embodiments, are CRAC inhibitors. In some embodiments, the CRAC inhibitor comprises a lanthanide. In some embodiments, the CRAC inhibitor comprises lanthanide trivalent ion. In some embodiments, the CRAC inhibitor comprises La³⁺ (lanthanum). In some embodiments, the CRAC inhibitor comprises Gd³⁺ (gadolinium). Each of these compounds is also an example of a Calcium channel inhibitor, or of a SOC inhibitor.

Disclosed herein, in some embodiments, are CRAC inhibitors. In some embodiments, the CRAC inhibitor comprises an imidazole. In some embodiments, the CRAC inhibitor comprises imidazole antimycotic SKF-96365. In some embodiments, the CRAC inhibitor comprises econazole. In some embodiments, the CRAC inhibitor comprises miconazole. Each of these compounds is also an example of a Calcium channel inhibitor, or of a SOC inhibitor.

Disclosed herein, in some embodiments, are CRAC inhibitors. In some embodiments, the CRAC inhibitor comprises a diphenylboronate. In some embodiments, the CRAC inhibitor comprises 2-Aminoethyldiphenyl borate (2-APB). In some embodiments, the CRAC inhibitor comprises DPB162-AE. In some embodiments, the CRAC inhibitor comprises DPB163-AE. Each of these compounds is also an example of a Calcium channel inhibitor, or of a SOC inhibitor.

Disclosed herein, in some embodiments, are CRAC inhibitors. In some embodiments, the CRAC inhibitor comprises a pyrazole. In some embodiments, the CRAC inhibitor comprises a bis(trifluoromethyl)pyrazole. In some embodiments, the CRAC inhibitor comprises BTP1. In some embodiments, the CRAC inhibitor comprises BTP2. In some embodiments, the CRAC inhibitor comprises YM-58483. In some embodiments, the CRAC inhibitor comprises BTP3. Each of these compounds is also an example of a Calcium channel inhibitor, or of a SOC inhibitor.

Disclosed herein, in some embodiments, are CRAC inhibitors. In some embodiments, the CRAC inhibitor comprises a Pyr compound. Disclosed herein, in some embodiments, are CRAC inhibitors. In some embodiments, the CRAC inhibitor comprises N-(4-(3,5-bis(trifluoromethyl)-1H-pyrazol-1-yl)phenyl)-4-methyl-1,2,3-thiadiazole-5-carboxamide (Pyr2/BTP2/YM58483). In some embodiments, the CRAC inhibitor comprises ethyl 1-(4-(2,3,3-trichloroacrylamido)phenyl)-5-(trifluoromethyl)-1H-pyrazole-4-carboxylate (Pyr3). In some embodiments, the CRAC inhibitor comprises N-(4-(3,5-bis(trifluoromethyl)-1H-pyrazol-1-yl)phenyl)-3-fluoroisonicotinamide (Pyr6). In some embodiments, the CRAC inhibitor comprises N-(4-(3,5-bis(trifluoromethyl)-1H-pyrazol-1-yl)phenyl)-4-methylbenzenesulfonamide (Pyr10). In some embodiments, the CRAC inhibitor comprises 2-aminoethoxy diphenylborate (2-APB). In some embodiments, the CRAC inhibitor comprises 2,2′-((((oxybis(methylene))bis(3,1-phenylene))bis(phenylboranediyl))bis(oxy))bis(ethan-1-amine) (DPB162-AE); 2,2′-((((oxybis(methylene))bis(4,1-phenylene))bis(phenylboranediyl))bis(oxy))bis(ethan-1-amine) (DPB163-AE). Each of these compounds is also an example of a Calcium channel inhibitor, or of a SOC inhibitor.

Disclosed herein, in some embodiments, are CRAC inhibitors. In some embodiments, the CRAC inhibitor comprises a GSK compound. In some embodiments, the CRAC inhibitor comprises GSK-5498A. In some embodiments, the CRAC inhibitor comprises GSK-5503A (2,6-difluoro-N-(1-(2-phenoxy benzyl)-1H-pyrazol-3-yl)benzamide). In some embodiments, the CRAC inhibitor comprises GSK-7975A (2,6-difluoro-N-(1-(4-hydroxy-2-(trifluoromethyl)benzyl)-1H-pyrazol-3-yl)benzamide). Each of these compounds is also an example of a Calcium channel inhibitor, or of a SOC inhibitor.

Disclosed herein, in some embodiments, are CRAC inhibitors. In some embodiments, the CRAC inhibitor comprises a polyunsaturated fatty acid (PUFA). In some embodiments, the CRAC inhibitor comprises an 18-C PUFA. In some embodiments, the CRAC inhibitor comprises linoleic acid. Each of these compounds is also an example of a Calcium channel inhibitor, or of a SOC inhibitor.

Disclosed herein, in some embodiments, are CRAC inhibitors. In some embodiments, the CRAC inhibitor comprises 1-phenyl-3-(1-phenylethyl) urea. In some embodiments, the CRAC inhibitor comprises a 1-phenyl-3-(1-phenylethyl) urea derivative. In some embodiments, the CRAC inhibitor comprises a 1-phenyl-3-(1-phenylethyl) urea derivative comprising Compound 22. In some embodiments, the CRAC inhibitor comprises a 1-phenyl-3-(1-phenylethyl) urea derivative comprising Compound 23. Each of these compounds is also an example of a Calcium channel inhibitor, or of a SOC inhibitor.

Disclosed herein, in some embodiments, are CRAC inhibitors. In some embodiments, the CRAC inhibitor comprises a cholestatic bile acid. In some embodiments, the CRAC inhibitor comprises taurolithocholic acid (TLCA; 2-[4-[(3R,5R,8R,9S,10S,13R,14S,17R)-3-hydroxy-10,13-dimethyl-2,3,4,5,6,7,8,9,11,12,14,15,16,17-tetradecahydro-1H-cyclopenta[a]phenanthren-17-yl]pentanoylamino]ethanesulfonic acid). In some embodiments, the CRAC inhibitor comprises lithocholic acid (LCA; (4R)-4-[(3R,5R,8R,9S,10S,13R,14S,17R)-3-Hydroxy-10,13-dimethyl-2,3,4,5,6,7,8,9,11,12,14,15,16,17-tetradecahydro-1H-cyclopenta[a]phenanthren-17-yl]pentanoic acid). In some embodiments, the CRAC inhibitor comprises cholic acid (CA; (R)-4-((3R,5S,7R,8R,9S,10S,12S,13R,14S,17R)-3,7,12-trihydroxy-10,13-dimethylhexadecahydro-1H-cyclopenta[a]phenanthren-17-yl) pentanoic acid). In some embodiments, the CRAC inhibitor comprises taurocholic acid (TCA; 2-{[(3a,5B,7a, 12a)-3,7,12-trihydroxy-24-oxocholan-24-yl]amino}ethanesulfonic acid)). Each of these compounds is also an example of a Calcium channel inhibitor, or of a SOC inhibitor.

Disclosed herein, in some embodiments, are CRAC inhibitors. In some embodiments, the CRAC inhibitor comprises FCC2121 (4-[3-(diphenylmethyl)-1,2,4-oxadiazol-5-yl]piperidinyl]piperidine). In some embodiments, the CRAC inhibitor comprises FCC2122 (3-(4-methyl-1,5-diphenyl-1H-pyrazol-3-yl)-2-phenylpropanoic acid). In some embodiments, the CRAC inhibitor comprises FC-2399 (2-(4-Chloro-phenyl)-3-[1-(4-chloro-phenyl)-5-methyl-1H-pyrazol-3-yl]-propionic acid). Each of these compounds is also an example of a Calcium channel inhibitor, or of a SOC inhibitor.

Disclosed herein, in some embodiments, are CRAC inhibitors. In some embodiments, the CRAC inhibitor comprises any one of N-[1-({2-Chloro-5-[(cyclopropylmethyl)oxy]phenyl}methyl)-1H-pyrazol-3-yl]-2,6-difluorobenzamide; N-{1-[(2,4-Dichlorophenyl)methyl]-1H-pyrazol-3-yl}-2,6-difluorobenzamide; 2-Bromo-N-{1-[(2,4-dichlorophenyl)methyl]-1H-pyrazol-3-yl}-6-fluorobenzamide; 2-Chloro-N-{1-[(2,4-dichlorophenyl)methyl]-1H-pyrazol-3-yl}-6-fluorobenzamide; 2,6-Dichloro-N-{1-[(2,4-dichlorophenyl)methyl]-1H-pyrazol-3-yl}benzamide; N-{1-[(2,4-dichlorophenyl)methyl]-1H-pyrazol-3-yl}-3,5-difluoro-4-pyridinecarboxamide; N-[1-({5-chloro-2-[(phenylmethyl)oxy]phenyl}methyl)-1H-pyrazol-3-yl]-2,6-25 difluorobenzamide; N-{1-[(2,6-dichlorophenyl)methyl]-1H-pyrazol-3-yl}-2,6-difluorobenzamide; N-[1-({5-chloro-2-[(2-methylpropyl)oxy]phenyl}methyl)-1H-pyrazol-3-yl]-2,6-difluorobenzamide; N-(1-{[2-bromo-5-(methyloxy)phenyl]methyl}-1H-pyrazol-3-yl)-2,6-difluorobenzamide; N-(1-{[5-chloro-2-(methyloxy)phenyl]methyl}-1H-pyrazol-3-yl)-2,6-difluorobenzamide; 2,6-Difluoro-N-(1-{[2-(phenyloxy)phenyl]methyl}-1H-pyrazol-3-yl)benzamide; N-[1-({5-bromo-2-[(phenylmethyl)oxy]phenyl}methyl)-1H-pyrazol-3-yl]-2,6-difluorobenzamide; 2,6-Difluoro-N-[1-({2-[(trifluoromethyl)oxy]phenyl}methyl)-1H-pyrazol-3-yl]benzamide; 2,6-Difluoro-N-(1-{[4-[(phenylmethyl)oxy]-2-(trifluoromethyl)phenyl]methyl}-1H-pyrazol-3-yl)benzamide; N-{1-[(2-Bromo-6-chlorophenyl)methyl]-1H-pyrazol-3-yl}-2,6-difluorobenzamide; 2,6-Difluoro-/V-[1-({2-[(phenylmethyl)oxy]phenyl}methyl)-1H-pyrazol-3-yl]benzamide; N/-[1-({2-chloro-5-[(2-methylpropyl)oxy]phenyl}methyl)-1H-pyrazol-3-yl]-2,6-difluorobenzamide; N-(1-{[4-[(cyclopropylmethyl)oxy]-2-(trifluoromethyl)phenyl]methyl}-1H-pyrazol-3-yl)-2,6-difluorobenzamide; 2,6-Difluoro-N-(1-{[4-iodo-2-(trifluoromethyl)phenyl]methyl}-1H-pyrazol-3-yl)benzamide; 2,6-Difluoro-N-(1-{[4-methyl-2-(trifluoromethyl)phenyl]methyl}-1H-pyrazol-3-yl)benzamide; N-(1-{[4-cyclopropyl-2-(trifluoromethyl)phenyl]methyl}-1H-pyrazol-3-yl)-2,6-difluorobenzamide; 2,6-Difluoro-N-{1-[(4-iodo-2-methylphenyl)methyl]-1H-pyrazol-3-yl}benzamide; N-(1-{[4-chloro-2-(trifluoromethyl)phenyl]methyl}-1H-pyrazol-3-yl)-2,6-difluorobenzamide; 2-Fluoro-N-(1-{[4-iodo-2-(trifluoromethyl)phenyl]methyl}-1H-pyrazol-3-yl)benzamide; 2-Chloro-N-(1-{[4-cyclopropyl-2-(trifluoromethyl)phenyl]methyl}-1H-pyrazol-3-yl)benzamide; N-(1-{[4-cyclopropyl-2-(trifluoromethyl)phenyl]methyl}-1H-pyrazol-3-yl)-2-fluorobenzamide; 2,6-Difluoro-N-(1-{[5-iodo-2-(trifluoromethyl)phenyl]methyl}-1H-pyrazol-3-yl)benzamide; 2,6-Difluoro-N-(1-{[2-fluoro-6-(trifluoromethyl)phenyl]methyl}-1H-pyrazol-3-yl)benzamide; or 2,6-Difluoro-N-(1-{[4-hydroxy-2-(trifluoromethyl)phenyl]methyl}-1H-pyrazol-3-yl)benzamide. Each of these compounds is also an example of a Calcium channel inhibitor, or of a SOC inhibitor.

Disclosed herein, in some embodiments, are CRAC inhibitors. In some embodiments, the CRAC inhibitor comprises any one of N-[4-(3,5-dicyclopropyl-1H-pyrazol-1-yl)phenyl]-1H-benzo[d]imidazole-6-carboxamide; N-[4-(3,5-dicyclopropyl-1H-pyrazol-1-yl)phenyl]-1H-benzo[d][1,2,3]triazole-6-carboxamide; N-[4-(3,5-dicyclopropyl-1H-pyrazol-1-yl)phenyl]quinoline-6-carboxamide; N-[4-(3,5-dicyclopropyl-1H-pyrazol-1-yl)phenyl]quinoxaline-6-carboxamide; 2-(1H-benzo[d]imidazol-1-yl)-N-[4-(3,5-dicyclopropyl-1H-pyrazol-1-yl)phenyl]acetamide; 2-(1H-benzo[d][1,2,3]triazol-1-yl)-N-[4-(3,5-dicyclopropyl-1H-pyrazol-1-yl)phenyl]acetamide; N-[4-(3,5-dicyclopropyl-1H-pyrazol-1-yl)phenyl]-2-(1H-indol-3-yl) acetamide; N-[4-(3,5-dicyclopropyl-1H-pyrazol-1-yl)phenyl]-2-(imidazo[1,2-a]pyridin-2-yl) acetamide; N-[4-(3,5-dicyclopropyl-1H-pyrazol-1-yl)phenyl]-2-(quinolin-6-yl) acetamide; N-[4-(3,5-dicyclopropyl-1H-pyrazol-1-yl)phenyl]-2-(quinolin-6-yl) acetamide; 2-(1H-benzo[d][1,2,3]triazol-1-yl)-N-(4-(3,5-dicyclopropyl-1H-pyrazol-1-yl)-3-fluorophenyl) acetamide; N-[4-(3,5-dicyclopropyl-1H-pyrazol-1-yl)-3-fluorophenyl]-2-(quinolin-6-yl) acetamide; N-[6-(3,5-dicyclopropyl-1H-pyrazol-1-yl) pyridin-3-yl]quinoline-6-carboxamide; N-[6-(3,5-dicyclopropyl-1H-pyrazol-1-yl) pyridin-3-yl]quinoxaline-6-carboxamide; 2-(1H-benzo[d][1,2,3]triazol-1-yl)-N-[6-(3,5-dicyclopropyl-1H-pyrazol-1-yl) pyridin-3-yl]acetamide; N-[6-(3,5-dicyclopropyl-1H-pyrazol-1-yl) pyridin-3-yl]-2-(quinolin-6-yl) acetamide; N-{4-[5-cyclopropyl-3-(trifluoromethyl)-1H-pyrazol-1-yl]phenyl}quinoline-6-carboxamide; N-{4-[5-cyclopropyl-3-(trifluoromethyl)-1H-pyrazol-1-yl]phenyl}quinoxaline-6-carboxamide; 2-(1H-benzo[d]imidazol-1-yl)-N-{4-[5-cyclopropyl-3-(trifluoromethyl)-1H-pyrazol-1-yl]phenyl]acetamide; 2-(1H-benzo[d][1,2,3]triazol-1-yl)-N-{4-[5-cyclopropyl-3-(trifluoromethyl)-1H-pyrazol-1-yl]phenyl}acetamide; 2-(2H-benzo[d][1,2,3]triazol-2-yl)-N-{4-[5-cyclopropyl-3-(trifluoromethyl)-1H-pyrazol-1-yl]phenyl}acetamide; 2-(3H-[1,2,3]triazolo[4,5-b]pyridin-3-yl)-N-{4-[5-cyclopropyl-3-(trifluoromethyl)-1H-pyrazol-1-yl]phenyl}acetamide; (S)-2-(3H-[1,2,3]triazolo[4,5-b]pyridin-3-yl)-N-{4-[5-cyclopropyl-3-(trifluoromethyl)-1H-pyrazol-1-yl]phenyl}propanamide; 2-(6-amino-9H-purin-9-yl)-N-{4-[5-cyclopropyl-3-(trifluoromethyl)-1H-pyrazol-1-yl]phenyl}acetamide; N-(4-(5-cyclopropyl-3-(trifluoromethyl)-1H-pyrazol-1-yl)phenyl)-2-(1,3-dimethyl-2,6-dioxo-2,3-dihydro-1H-purin-7 (6H)-yl) acetamide; N-{4-[5-cyclopropyl-3-(trifluoromethyl)-1H-pyrazol-1-yl)phenyl)-2-(imidazo[1,2-a]pyridin-2-yl) acetamide; N-{4-[5-cyclopropyl-3-(trifluoromethyl)-1H-pyrazol-1-yl]phenyl}-2-(quinolin-6-yl) acetamide; N-{4-[5-cyclopropyl-3-(trifluoromethyl)-1H-pyrazol-1-yl]phenyl}-2-(quinolin-6-yl) propanamide; N-{4-[5-cyclopropyl-3-(trifluoromethyl)-1H-pyrazol-1-yl]-3-fluorophenyl}-1H-benzo[d][1,2,3]triazole-6-carboxamide; 2-(1H-benzo[d][1,2,3]triazol-1-yl)-N-{4-[5-cyclopropyl-3-(trifluoromethyl)-1H-pyrazol-1-yl]-3-fluorophenyl}acetamide; N-{6-[5-cyclopropyl-3-(trifluoromethyl)-1H-pyrazol-1-yl]pyridin-3-yl}-1H-benzo[d][1,2,3]triazole-5-carboxamide; 2-(1H-benzo[d][1,2,3]triazol-1-yl)-N-{6-[5-cyclopropyl-3-(trifluoromethyl)-1H-pyrazol-1-yl]pyridin-3-yl}acetamide; 2-(2H-benzo[d][1,2,3]triazol-2-yl)-N-{6-[5-cyclopropyl-3-(trifluoromethyl)-1H-pyrazol-1-yl]pyridin-3-yl}acetamide; N-{6-[5-cyclopropyl-3-(trifluoromethyl)-1H-pyrazol-1-yl]pyridin-3-yl}-2-(quinolin-6-yl) acetamide; 2-(1H-benzo[d][1,2,3]triazol-1-yl)-N-{6-[4-chloro-5-cyclopropyl-3-(trifluoromethyl)-1H-pyrazol-1-yl]pyridin-3-yl}acetamide; 4-[5-cyclopropyl-3-(trifluoromethyl)-1H-pyrazol-1-yl]-3-fluoro-N-(quinolin-6-ylmethyl)benzamide; or 1-[4-(3,5-dicyclopropyl-1H-pyrazol-1-yl)phenyl]-3-(quinolin-6-yl) urea. Each of these compounds is also an example of a Calcium channel inhibitor, or of a SOC inhibitor.

Disclosed herein, in some embodiments, are CRAC inhibitors. In some embodiments, the CRAC inhibitor comprises any one of 4-[6-(2-chloro-6-fluoro-phenyl)-5H-pyrrolo[3,2-d]pyrimidin-2-yl]-3,N,N-trimethyl-benzenesulfonamide; 6-(2-Chloro-phenyl)-2-(2-methyl-5-trifluoromethyl-2H-pyrazol-3-yl)-5H-pyrrolo[2,3-b]pyrazine; 4-[6-(2-Chloro-phenyl)-5H-pyrrolo[2,3-b]pyrazin-2-yl]-3-methyl-benzoic acid methyl ester; 4-(6-(2-Chlorophenyl)-5H-pyrrolo[2,3-b]pyrazin-2-yl)-N,N,3-trimethylbenzenesulfonamide; 6-(2-chloro-6-fluorophenyl)-2-(1-methyl-3-(trifluoromethyl)-1H-pyrazol-5-yl)-5H-pyrrolo[2,3-b]pyrazine; 6-Cyclohexyl-2-(1-methyl-3-(trifluoromethyl)-1H-pyrazol-5-yl)-5H-pyrrolo[2,3-b]pyrazine; or 4-(6-Cyclohexyl-5H-pyrrolo[2,3-b]pyrazin-2-yl)-N,N,3-trimethylbenzenesulfonamide. Each of these compounds is also an example of a Calcium channel inhibitor, or of a SOC inhibitor.

Disclosed herein, in some embodiments, are CRAC inhibitors. In some embodiments, the CRAC inhibitor comprises any one of 2,6-Difluoro-N-(6-(5-(4-methyl-5-oxo-4,5-dihydro-1,3,4-oxadiazol-2-yl)-3-(trifluoromethyl)-1H-pyrazol-1-yl) pyridin-3-yl)benzamide; 2-Fluoro-6-methyl-N-(6-(5-(4-methyl-5-oxo-4,5-dihydro-1,3,4-oxadiazol-2-yl)-3-(trifluoromethyl)-1H-pyrazol-1-yl) pyridin-3-yl)benzamide; 5-(3-Cyclopropyl-1-(5-(2,6-difluorobenzyl)amino)pyridin-2-yl)-1H-pyrazol-5-yl)-3-methyl-1,3,4-oxadiazol-2 (3H)-one; N-(6-(3-(Difluoromethyl)-5-(4-methyl-5-oxo-4,5-dihydro-1,3,4-oxadiazol-2-yl)-1H-pyrazol-1-yl) pyridin-3-yl)-2,6-difluorobenzamide; 5-(1-(5-(2,6-Difluorobenzyl)amino)pyridin-2-yl)-5-(fluoromethyl)-1H-pyrazol-3-yl)-3-methyl-1,3,4-oxadiazol-2 (3H)-one; Methyl 3-(1-(5-((2,6-difluorobenzyl) amino)pyridin-2-yl)-5-(trifluoromethyl)-1H-pyrazol-3-yl)-5-methyl-4,5-dihydroisoxazole-5-carboxylate; Methyl 3-(1-(5-(2-chloro-6-fluorobenzyl)amino)pyridin-2-yl)-5-(trifluoromethyl)-1H-pyrazol-3-yl)-5-methyl-4,5-dihydroisoxazole-5-carboxylate; 2,6-Difluoro-W-(6-(3-(4-methyl-5-oxo-4,5-dihydro-1,3,4-oxadiazol-2-yl)-5-(trifluoromethyl)-1H-pyrazol-1-yl) pyridin-3-yl)benzamide; 2-Chloro-6-fluoro-N-(6-(3-(4-methyl-5-oxo-4,5-dihydro-1,3,4-oxadiazol-2-yl)-5-(trifluoromethyl)-\H-pyrazol-1-yl) pyridin-3-yl)benzamide; 2-Fluoro-6-methyl-/v-(6-(3-(4-methyl-5-oxo-4,5-dihydro-1,3,4-oxadiazol-2-yl)-5-(trifluoromethyl)-1H-pyrazol-1-yl) pyridin-3-yl)benzamide; N-(6-(5-(Difluoromethyl)-3-(4-methyl-5-oxo-4,5-dihydro-1,3,4-oxadiazol-2-yl)-1H-pyrazol-1-yl) pyridin-3-yl)-2,6-difluorobenzamide; 5-(1-(5 ((2,6-Difluorobenzyl) amino)pyridin-2-yl)-5-(difluoromethyl)-1H-pyrazol-3-yl)-3-methyl-1,3,4-oxadiazol-2 (3H)-one; 5-(1-(5-((2,6-Difluorobenzyl)amino)pyridin-2-yl)-3-(difluoromethyl)-1H-pyrazol-5-yl)-3-methyl-1,3,4-oxadiazol-2 (3H)-one; N-(6-(3-(5,5-Dimethyl-4-oxo-4,5-dihydroisoxazol-3-yl)-5-(trifluoromethyl)-1H-pyrazol-1-yl) pyridin-3-yl)-2,6-difluorobenzamide; 2-Chloro-N-(6-(3-(5,5-dimethyl-4-oxo-4,5-dihydroisoxazol-3-yl)-5-(trifluoromethyl)-1H-pyrazol-1-yl) pyridin-3-yl)-6-fluorobenzamide; 2,6-Difluoro-N-(6-(1′,4′,4,-trimethyl-5′-oxo-5-(trifluoromethyl)-4′,5′-dihydro-1H,1H′-[3,3′-bipyrazol]-1-yl) pyridin-3-yl)benzamide; 2-Chloro-6-fluoro-N-(6-(1,4,4′-trimethyl-5′-oxo-5-(trifluoromethyl)-4′,5,-dihydro-1H,1′H-[3,3′-bipyrazol]-1-yl) pyridin-3-yl)benzamide; 2-Fluoro-6-methyl-N-(6-(1,4,4′-trimethyl-5′-oxo-5-(trifluoromethyl)-4′,5′-dihydro-1H,1′H-[3,3′-bipyrazol]-1-yl) pyridin-3-yl)benzamide; 2,6-Difluoro-N-(6-(3-(4-methyl-5-oxo-4,5-dihydro-1,2,4-oxadiazol-3-yl)-5-(trifluoromethyl)-1H-pyrazol-1-yl) pyridin-3-yl)benzamide; N-(6-(3-(4-Acetyl-5,5-dimethyl-4,5-dihydro-1,3,4-oxadiazol-2-yl)-5-trifluoromethyl)-1H-pyrazol-1-yl) pyridin-3-yl)-2,6-difluorobenzamide; N-(6-(3-(4,4-Dimethyl-4,5-dihydrooxazol-2-yl)-5-(trifluoromethyl)-1H-pyrazol-1-yl) pyridin-3-yl)-2,6-difluorobenzamide; 5-(1-(5-(2,6-Difluorobenzyl)amino)pyridin-2-yl)-5-(trifluoromethyl)-1H-pyrazol-3-yl)-3-methyl-1,3,4-oxadiazol-2 (3H)-one; 5-(1-(5-((2-Chloro-6-fluorobenzyl)amino)pyridin-2-yl)-5-(trifluoromethyl)-1H-pyrazol-3-yl)-3-methyl-1,3,4-oxadiazol-2 (3iy)-one; 1′454 (2,6-Difluorobenzyl)amino)pyridin-2-yl)-1,4,4-trimethyl-5′-(trifluoromethyl)-1H,1′H-[3,3′-bipyrazol]-5 (4H)-one; 1′-(5-(2-Chloro-6-fluorobenzyl)amino)pyridin-2-yl)-1,4,4-trimethyl-5′-(trifluoromethyl)-1H,1′H-[3,3′-bipyrazol]-5 (4H)-one; 3-(1-(5-(2,6-Difluorobenzyl)amino)pyridin-2-yl)-5-(trifluoromethyl)-1H-pyrazol-3-yl)-4-methyl-1,2,4-oxadiazol-5 (4H)-one; 1-(5-(1-(5-(2,6-Difluorobenzyl)amino)pyridin-2-yl)-5-(trifluoromethyl)-1H-pyrazol-3-yl)-2,2-dimethyl-1,3,4-oxadiazol-3 (2H)-yl) ethanone; N-(2,6-Difluorobenzyl)-6-(3-(4,4-dimethyl-4,5-dihydrooxazol-2-yl)-5-(trifluoromethyl)-1H-pyrazol-1-yl) pyridin-3-amine; N-(6-(5-Cyclopropyl-3-(4-methyl-5-oxo-4,5-dihydro-1,3,4-oxadiazol-2-yl)-1H-pyrazol-1-yl) pyridin-3-yl)-2,6-difluorobenzamide; N-(6-(3-Cyclopropyl-5-(4-methyl-5-oxo-4,5-dihydro-1,3,4-oxadiazol-2-yl)-1H-pyrazol-1-yl) pyridin-3-yl)-2,6-difluorobenzamide; 2,6-Difluoro-N-(6-(5-methyl-3-(4-methyl-5-oxo-4,5-dihydro-1,3,4-oxadiazol-2-yl)-1H-pyrazol-1-yl) pyridin-3-yl)benzamide; 5-(1-(5-((2,6-Difluorobenzyl)amino)pyridin-2-yl)-5-methyl-1H-pyrazol-3-yl)-3-methyl-1,3,4-oxadiazol-2 (3H)-one; (3-(I-(5-((2,6-Difluorobenzyl)amino)pyridin-2-yl)-5-(trifluoromethyl)-1H-pyrazol-3-yl)-5-methyl-4,5-dihydroisoxazol-5-yl) methanol; (3-(1-(5-((2-Chloro-6-fluorobenzyl)amino)pyridin-2-yl)-5-(trifluoromethyl)-1H-pyrazol-3-yl)-5-methyl-4,5-dihydroisoxazol-5-yl) methanol; Methyl 3-(1-(5-(2,6-difluorobenzamido)pyridin-2-yl)-5-(trifluoromethyl)-1H-pyrazol-3-yl)-5-methyl-4,5-dihydroisoxazole-5-carboxylate; 2,6-Difluoro-N-(6-(3-(5-(hydroxymethyl)-5-methyl-4,5-dihydroisoxazol-3-yl)-5-(trifluoromethyl)-1H-pyrazol-1-yl) pyridin-3-yl)benzamide; 3-(1-(5-(2,6-Difluorobenzamido)pyridin-2-yl)-5-(trifluoromethyl)-1H-pyrazol-3-yl)-5-methyl-4,5-dihydroisoxazole-5-carboxamide; 2,6-Difluoro-N-(5-(3-(4-methyl-5-oxo-4,5-dihydro-1,3,4-oxadiazol-2-yl)-5-(trifluoromethyl)-1H-pyrazol-1-yl) pyridin-2-yl)benzamide; 2-Chloro-6-fluoro-N-(5-(3-(4-methyl-5-oxo-4,5-dihydro-1,3,4-oxadiazol-2-yl)-5-(trifluoromethyl)-1H-pyrazol-1-yl) pyridin-2-yl)benzamide; 2-Fluoro-6-methyl-N-(5-(3-(4-methyl-5-oxo-4,5-dihydro-1,3,4-oxadiazol-2-yl)-5-(trifluoromethyl)-1H-pyrazol-1-yl) pyridin-2-yl)benzamide; 2-Fluoro-N-(5-(3-(4-methyl-5-oxo-4,5-dihydro-1,3,4-oxadiazol-2-yl)-5-(trifluoromethyl)-1H-pyrazol-1-yl) pyridin-2-yl)benzamide; 2,3-Difluoro-N-(5-(3-(4-methyl-5-oxo-4,5-dihydro-1,3,4-oxadiazol-2-yl)-5-(trifluoromethyl)-1H-pyrazol-1-yl) pyridin-2-yl)benzamide; 2,4,5-Trifluoro-N-(5-(3-(4-methyl-5-oxo-4,5-dihydro-1,3,4-oxadiazol-2-yl)-5-(trifluoromethyl)-1H-pyrazol-1-yl) pyridin-2-yl)benzamide; 2,3,4-Trifluoro-N-(5-(3-(4-methyl-5-oxo-4,5-dihydro-1,3,4-oxadiazol-2-yl)-5-(trifluoromethyl)-1H-pyrazol-1-yl) pyridin-2-yl)benzamide; 2,4-Difluoro-N-(5-(3-(4-methyl-5-oxo-4,5-dihydro-1,3,4-oxadiazol-2-yl)-5-(trifluoromethyl)-1H-pyrazol-1-yl) pyridin-2-yl)benzamide; 2,3-Dimethyl-N-(5-(3-(4-methyl-5-oxo-4,5-dihydro-1,3,4-oxadiazol-2-yl)-5-(trifluoromethyl)-1H-pyrazol-1-yl) pyridin-2-yl)benzamide; 2-Chloro-N-(5-(3-(4-methyl-5-oxo-4,5-dihydro-1,3,4-oxadiazol-2-yl)-5-(trifluoromethyl)-1H-pyrazol-1-yl) pyridin-2-yl)benzamide; 2-Methyl-N-(5-(3-(4-methyl-5-oxo-4,5-dihydro-1,3,4-oxadiazol-2-yl)-5-(trifluoromethyl)-1H-pyrazol-1-yl) pyridin-2-yl)benzamide; 4-Ethyl-N-(5-(3-(4-methyl-5-oxo-4,5-dihydro-1,3,4-oxadiazol-2-yl)-5-(trifluoromethyl)-1//-pyrazol-1-yl) pyridin-2-yl)benzamide; N-(5-(3-(4-Methyl-5-oxo-4,5-dihydro-13,4-oxadiazol-2-yl)-5-(trifluoromethyl)-1H-pyrazol-1-yl) pyridin-2-yl)-2-naphthamide; 5-(1-(6-((2,6-Difluorobenzyl)amino)pyridin-3-yl)-5-(trifluoromethyl)-1H-pyrazol-3-yl)-3-methyl-1,3,4-oxadiazol-2 (3H)-one; 5-(1-(6-((2-Chloro-6-fluorobenzyl)amino)pyridin-3-yl)-5-(trifluoromethyl)-1H-pyrazol-3-yl)-3-methyl-1,3,4-oxadiazol-2 (3H)-one; 5-(1-(6-(2-Fluoro-6-methylbenzyl)amino)pyridm-3-yl)-5-(trifluoromethyl)-1H-pyrazol-3-yl)-3-methyl-1,3,4-oxadiazol-2 (3H)-one; N-(2,6-Difluorophenyl)-6-(3-(4-methyl-5-oxo-4,5-dihydro-1,3,4-oxadiazol-2-yl)-5-(trifluoromethyl)-1H-pyrazol-1-yl) nicotinamide; or N-(2-Chloro-6-fluorophenyl)-6-(3-(4-methyl-5-oxo-4,5-dihydro-1,3,4-oxadiazol-2-yl)-5-(trifluoromethyl)-1H-pyrazol-1-yl) nicotinamide. Each of these compounds is also an example of a Calcium channel inhibitor, or of a SOC inhibitor.

Disclosed herein, in some embodiments, are Calcium channel inhibitors, SOC inhibitors, or CRAC inhibitors. In some embodiments, the Calcium channel inhibitor, SOC inhibitor, or CRAC inhibitor comprises a small molecule such as a small molecule that interferes with the Calcium channel's activity, the SOC channel's activity, or the CRAC channel's activity. In some embodiments, the Calcium channel inhibitor, SOC inhibitor, or CRAC inhibitor comprises a polypeptide such as a mutated or nonfunctional form of a component of a Calcium channel, of a SOC channel, or of a CRAC channel that may interfere with the Calcium channel's activity, the SOC channel's activity, or the CRAC channel's activity.

### Further Forms of Compounds

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by the forming diastereomeric and separation by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

In some situations, compounds may exist as tautomers. All tautomers are included within the formulas described herein.

The methods and compositions described herein include the use of amorphous forms as well as crystalline forms (also known as polymorphs). The compounds described herein may be in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In some embodiments, compounds described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

To produce a prodrug, a pharmaceutically active compound is modified such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. In some embodiments, by virtue of knowledge of pharmacodynamic processes and drug metabolism in vivo, once a pharmaceutically active compound is determined, prodrugs of the compound are designed. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Saulnier et al., (1994), Bioorganic and Medicinal Chemistry Letters, Vol. 4, p. 1985; Rooseboom et al., Pharmacological Reviews, 56:53-102, 2004; Miller et al., J. Med. Chem. Vol. 46, no. 24, 5097-5116, 2003; Aesop Cho, “Recent Advances in Oral Prodrug Discovery”, Annual Reports in Medicinal Chemistry, Vol. 41, 395-407, 2006).

Prodrug forms of the herein described compounds, wherein the prodrug is metabolized in vivo to produce a compound as set forth herein, are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. Prodrugs may be designed as reversible drug derivatives, for use as modifiers to enhance drug transport to site-specific tissues. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure).

Sites on the aromatic ring portion of compounds described herein can be susceptible to various metabolic reactions, therefore incorporation of appropriate substituents on the aromatic ring structures, such as, by way of example only, halogens can reduce, minimize or eliminate this metabolic pathway.

The compounds described herein may be labeled isotopically (e.g. with a radioisotope) or by other means, including, but not limited to, the use of chromophores or fluorescent moieties, bioluminescent labels, photoactivatable or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, 2H, 3H, 13C, 14C, 15N, 18O, 17O, 35S, 18F, 36Cl, respectively. Certain isotopically-labeled compounds described herein, for example those into which radioactive isotopes such as 3H and 14C are incorporated, are useful in drug and/or substrate tissue distribution assays. Further, substitution with isotopes such as deuterium, i.e., 2H, can afford certain therapeutic advantages resulting from greater metabolic stability, such as, for example, increased in vivo half-life or reduced dosage requirements.

In additional or further embodiments, the compounds described herein are metabolized upon administration to an organism in need to produce a metabolite that is then used to produce a desired effect, including a desired therapeutic effect.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

In some embodiments, compounds described herein, are in various forms, including but not limited to, amorphous forms, milled forms, injectable emulsion forms, and nano-particulate forms. In addition, compounds described herein include crystalline forms, also known as polymorphs. Polymorphs include the different crystal packing arrangements of the same elemental composition of a compound. Polymorphs usually have different X-ray diffraction patterns, melting points, density, hardness, crystal shape, optical properties, stability, and solubility. Various factors such as the recrystallization solvent, rate of crystallization, and storage temperature may cause a single crystal form to dominate.

The screening and characterization of the pharmaceutically acceptable salts, polymorphs and/or solvates may be accomplished using a variety of techniques including, but not limited to, thermal analysis, x-ray diffraction, spectroscopy, vapor sorption, and microscopy. Thermal analysis methods address thermo chemical degradation or thermo physical processes including, but not limited to, polymorphic transitions, and such methods are used to analyze the relationships between polymorphic forms, determine weight loss, to find the glass transition temperature, or for excipient compatibility studies. Such methods include, but are not limited to, Differential scanning calorimetry (DSC), Modulated Differential Scanning calorimetry (MDCS), Thermogravimetric analysis (TGA), and Thermogravi-metric and Infrared analysis (TG/IR). X-ray diffraction methods include, but are not limited to, single crystal and powder diffractometers and synchrotron sources. The various spectroscopic techniques used include, but are not limited to, Raman, FTIR, UV-VIS, and NMR (liquid and solid state). The various microscopy techniques include, but are not limited to, polarized light microscopy, Scanning Electron Microscopy (SEM) with Energy Dispersive X-Ray Analysis (EDX), Environmental Scanning Electron Microscopy with EDX (in gas or water vapor atmosphere), IR microscopy, and Raman microscopy.

Throughout the specification, groups and substituents thereof can be chosen to provide stable moieties and compounds.

### Synthesis of Compounds

In some embodiments, the synthesis of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, Fischer Scientific (Fischer Chemicals), and Acros Organics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4th Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999) (all of which are incorporated by reference for such disclosure).

PaO₂/FiO₂ Ratio

The PaO₂/FiO₂ (P/F) ratio can be a useful biomarker for patient selection for inflammatory lung injury diseases such as acute lung injury (ALI) and acute respiratory distress syndrome (ARDS). A “normal” P/F ratio is considered greater than or about 300, while lower ratio numbers indicate respiratory issues, eg P/F ratio of 200-300 is mild respiratory failure, 100-200 is moderate respiratory failure, and less than 100 is severe respiratory failure by the Berlin criteria (https://www.mdcalc.com/berlin-criteria-acute-respiratory-distress-syndrome). Further, selection of patients using P/F ratios as a biomarker can help determine responders and non-responders for the methods described herein. Non-responders can be considered patients with P/F ratios of less than 100 after 2 doses of the therapeutic agents. In such cases, non-responders should receive a third dose, and up to six total doses. Patients with P/F ratios less than 100 showed mortality rates of up to 65%, while patients with P/F ratios greater than 100 showed mortality rates of less than 10%.

In some embodiments, the present disclosure provides a method for treating acute lung injury (ALI), acute respiratory distress syndrome (ARDS), COVID-19 pneumonia, bacterial pneumonia, viral pneumonia, acute pancreatitis, or a combination thereof, in a subject, comprising administering a therapeutically effective amount of an intracellular Calcium signaling inhibitor to said subject, wherein the subject has a PaO₂/FiO₂ (P/F) ratio of less than or equal to about 300, and wherein the subject has been administered with at least two doses of the intracellular Calcium signaling inhibitor. In some embodiments, the present disclosure provides a method for treating acute lung injury (ALI), acute respiratory distress syndrome (ARDS), COVID-19 pneumonia, bacterial pneumonia, viral pneumonia, acute pancreatitis, or a combination thereof, in a subject, comprising administering a therapeutically effective amount of an intracellular Calcium signaling inhibitor to said subject, wherein the subject has a PaO₂/FiO₂ (P/F) ratio of less than or equal to about 150, and wherein the subject has been administered with at least two doses of the intracellular Calcium signaling inhibitor.

In some embodiments, the present disclosure provides method for treating acute lung injury (ALI), acute respiratory distress syndrome (ARDS), COVID-19 pneumonia, bacterial pneumonia, viral pneumonia, acute pancreatitis, or a combination thereof, in a subject, comprising administering a therapeutically effective amount of a corticosteroid and/or immunosuppressive drug and an intracellular Calcium signaling inhibitor to said subject, wherein the subject has a PaO₂/FiO₂ (P/F) ratio of less than or equal to about 300. In some embodiments, the present disclosure provides a method for treating acute lung injury (ALI), acute respiratory distress syndrome (ARDS), COVID-19 pneumonia, bacterial pneumonia, viral pneumonia, acute pancreatitis, or a combination thereof, in a subject, comprising administering a therapeutically effective amount of a corticosteroid and/or immunosuppressive drug and an intracellular Calcium signaling inhibitor to said subject, wherein the subject has a PaO₂/FiO₂ (P/F) ratio of less than or equal to about 150.

The P/F ratio of subjects suitable for the methods of the present disclosure can be any suitable P/F ratio known by one of skill in the art. In some embodiments, the subject has P/F ratio of less than or equal to about 200. In some embodiments, the subject has P/F ratio of less than or equal to about 150. In some embodiments, the subject has P/F ratio of less than or equal to about 100. In some embodiments, the subject has a P/F ratio of about 50 to about 150. In some embodiments, wherein the subject has a P/F ratio of about 50 to about 125. In some embodiments, wherein the subject has a P/F ratio of about 50 to about 100. In some embodiments, the subject has a P/F ratio of about 75 to about 100. In some embodiments, the subject has a P/F ratio of about 75 to about 150. In some embodiments, the subject shows modulation of the P/F ratio after administration of the intracellular Calcium signaling inhibitor. In some embodiments, the subject shows increase in the P/F ratio after administration of the intracellular Calcium signaling inhibitor.

The recovery rate of the subject after administration of the intracellular Calcium signaling inhibitor can be any suitable value known by one of skill in the art. The recovery rate of the subject is also applicable to combination therapy, wherein the intracellular Calcium signaling inhibitor is administered with an effective amount of a corticosteroid or immunosuppressive drug described herein. In some embodiments, the subject has a recovery rate of greater than about 35%. In some embodiments, the subject has a recovery rate of greater than about 50%. In some embodiments, the subject has a recovery rate of greater than about 75%. In some embodiments, the subject has a recovery rate of greater than about 90%. In some embodiments, the subject has a recovery rate of about 35% to about 100%. In some embodiments, the subject has a recovery rate of about 35% to about 95%. In some embodiments, the subject has a recovery rate of about 50% to about 100%. In some embodiments, the subject has a recovery rate of about 50% to about 95%.

In some embodiments, the subject is further administered oxygen therapy. In some embodiments, the oxygen therapy is administered by rebreather mask, venturi mask, high-flow nasal cannula (HFNC) or non-invasive ventilation (NIV). In some embodiments, the oxygen therapy is administered by high-flow nasal cannula (HFNC) or non-invasive ventilation (NIV).

The administration of the intracellular Calcium signaling inhibitor can be administered any suitable dose to improve the P/F ratio of the subject. In some embodiments, if the subject is administered two doses, and the P/F ratio is less than or about 100, then the subject is considered a non-responder and should receive a third dose of the intracellular Calcium signaling inhibitor, and at least a fourth, and potentially fifth, or sixth dose. In some embodiments, the subject has a P/F ratio of less than or about 100 after receiving two doses of the intracellular Calcium signaling inhibitor, and receives at least three, four, five, or six total doses. In some embodiments, the subject has a P/F ratio of less than or about 100 after receiving two doses of the intracellular Calcium signaling inhibitor, and in addition to a third dose receives four, five, or six total doses. In some embodiments, the subject has a P/F ratio of less than or about 100 after receiving two doses of the intracellular Calcium signaling inhibitor, and receives a third dose, and at least a fourth, fifth, or sixth doses. In some embodiments, the intracellular Calcium signaling inhibitor is administered with at least three doses. In some embodiments, the intracellular Calcium signaling inhibitor is administered with six total doses. In some embodiments, the intracellular Calcium signaling inhibitor is administered with at least three, four, five, six, seven, eight, nine, or ten doses.

In some embodiments, a second dose of the intracellular Calcium signaling inhibitor is administered about 24 hours after a first dose. In some embodiments, a third dose of the intracellular Calcium signaling inhibitor is administered about 48 hours after a first dose. In some embodiments, the concentration of the first dose is greater than the concentration of the second and third dose. In some embodiments, the concentration of the first dose is the same as the concentration of the second and third dose.

The intracellular Calcium signaling inhibitor can be administered in any suitable concentration known by one of skill in the art. In some embodiments, the intracellular Calcium signaling inhibitor is administered in a concentration of about 0.1 mg/kg to about 5 mg/kg. In some embodiments, the intracellular Calcium signaling inhibitor is administered in a concentration of about 0.5 mg/kg to about 3 mg/kg. In some embodiments, the intracellular Calcium signaling inhibitor is administered in a concentration of about 0.5 mg/kg to about 2.5 mg/kg. In some embodiments, the intracellular Calcium signaling inhibitor is administered in a concentration of about 1 mg/kg to about 2.0 mg/kg. In some embodiments, the intracellular Calcium signaling inhibitor is administered in a concentration of about 1.5 mg/kg to about 2.0 mg/kg. In some embodiments, the intracellular Calcium signaling inhibitor is administered in a concentration of about 1.6 mg/kg to about 2.0 mg/kg. In some embodiments, the first dose is administered in a concentration of about 2.0 mg/kg, and the second and third dose is administered in a concentration of about 1.6 mg/kg. In some embodiments, the concentration of the intracellular Calcium signaling inhibitor is increased or decreased when the subject's P/F ratio similar to the P/F ratio prior to administration.

### Acute Lung Injury (ALI) and its Severe Manifestation, Acute Respiratory Distress Syndrome (ARDS)

ALI and ARDS are deadly and complex respiratory complications that includes various pathogenic factors such as aspiration of gastric contents, microbial infection, sepsis and trauma. There are two major pathological features of ALI/ARDS; edema and neutrophil accumulation in the lung tissue. Initial inflammatory stimuli disrupt lung endothelial and/or epithelial barrier and induce extravasation of protein rich fluid resulting in lung edema. These stimuli also cause neutrophil infiltration into the interstitium and alveolar airspace. Infiltrated neutrophils injure lung parenchymal cells by secreting elastase and reactive oxygen species, inducing the further production of pro-inflammatory cytokines and activation of inflammatory cells. These physical and chemical tissue damages lead to the impairment of air exchange and severe respiratory dysfunction.

The innate immune response plays a role in the pathophysiology of ALI/ARDS. Multiple immunologic processes involving neutrophils, macrophages, and dendritic cells involve in mediating tissue injury. Inflammatory insults, either locally from the lungs or systemically from extrapulmonary sites, affect bronchial epithelium, alveolar macrophages, and vascular endothelium, causing accumulation of protein-rich edema fluid into the alveoli and, subsequently, hypoxemia due to impaired gas exchange. Alveolar macrophages play a central role in orchestrating inflammation, as well as the resolution of ARDS. Once alveolar macrophages are stimulated, they recruit neutrophils and circulating macrophages to the pulmonary sites of injury. These cells are involved in the elaboration of a diverse array of bioactive mediators, including proteases, reactive oxygen species, eicosanoids, phospholipids, and cytokines, that perpetuate inflammatory responses. One profound effect of these mediators is to damage or induce distal cell death, specifically alveolar type 2 epithelial cells. These cells serve vital functions by synthesizing and secreting pulmonary surfactant, which is an indispensable material that lines the inner lung surface to lower alveolar surface tension. Type 2 cells also actively partake in ion transport to control lung fluid. Together, these inflammatory events lead to histological changes typical of an acute exudative phase that results in significant impairment of lung mechanics and gas exchange. During the initial inflammatory and/or resolution phases of ARDS, alveolar macrophages also coordinate in a paracrine manner to interact with other cells, including epithelial cells, lymphocytes, and mesenchymal stem cells, which can result in augmentation of the inflammatory response or accentuation of tissue injury. Prolonged M1 (classically activated macrophages) or M2 (alternatively activated macrophages) phenotypes appear to be associated with nonhealing chronic ARDS/ALI. ARDS/ALI is a systemic inflammatory disease with bidirectional involvement between the lungs and other organ systems, rather than a local pulmonary process. Inflammatory cytokines, such as IL-1β, TNF-α, IL-6, and IL-8, are elevated both in bronchoalveolar lavage fluid and circulating plasma in ARDS subjects.

Bacterial and viral infections, such as Coronavirus (COVID-19) infection, can lead to ALI/ARDS. During infection, circulating bacterial and/or viral products and endogenous cytokines (e.g., IL-2, IL-6) stimulate the endothelium, setting off a cascade of vascular activation, including the increased expression of vascular adhesion molecules and regional increases in endothelial permeability.

In some embodiments, the ALI or ARDS is associated with a symptom or marker associated with the ALI or ARDS. For example, the ALI or ARDS may be associated with an increased expression of Stim1, Orai1, or PKCα, increased cellular Ca²⁺ uptake or Ca²⁺ levels, an increase in AMPK activation, an increase in ACC or PLC phosphorylation, Na,K-ATPase downregulation, alveolar epithelial dysfunction, an increase in edema, an increase in a lung wet/dry weight ratio, an increase in a BALF protein level, or an increase in endothelial permeability, in a subject (e.g. in a subject's lungs). In some embodiments, the ALI or ARDS comprises lung inflammation and/or endothelial cell dysfunction in a subject, for example, in a subject's lungs. In some embodiments, the inflammation is caused or contributed by endothelial cell dysfunction. In some embodiments, the ALI comprises pulmonary inflammation. In some embodiments, the ARDS comprises pulmonary inflammation. In some embodiments, the pulmonary inflammation is caused or contributed by pulmonary endothelial cell dysfunction. In some embodiments, the ALI comprises pulmonary endothelial cell dysfunction. In some embodiments, the ALI or ARDS comprises lung damage. In some embodiments, the ALI or ARDS comprises a cough. In some embodiments, the ALI or ARDS comprises a dry cough. In some embodiments, the ALI or ARDS comprises a fever or high temperature. In some embodiments, the ALI or ARDS comprises a shortness of breath. In some embodiments, the ALI or ARDS comprises a need for oxygen support. In some embodiments, the ALI or ARDS comprises a need for low flow oxygen. In some embodiments, the ALI or ARDS comprises a need for high flow oxygen. In some embodiments, the ALI or ARDS comprises a cytokine storm. In some embodiments, the ALI or ARDS comprises pulmonary endothelial damage. In some embodiments, the ALI or ARDS comprises CRAC channel overactivation. In some embodiments, the ALI or ARDS comprises an increase in intracellular calcium. In some embodiments, the ARDS comprises pulmonary endothelial cell dysfunction. In some embodiments, administration of a compound described herein, such as a CRAC inhibitor, may reduce, prevent, or reverse any of these symptoms.

In some embodiments, the ALI or ARDS comprises pneumonia. In some embodiments, the ALI comprises pneumonia. In some embodiments, the ARDS comprises pneumonia. In some embodiments, the pneumonia comprises a pneumonia stage such as a consolidation, red hepatization, grey hepatization, or resolution. In some embodiments, the pneumonia comprises a buildup of fluid in a subject's lung. In some embodiments, the pneumonia results in a symptom such as hypoxia or reduced blood oxygenation in a subject.

In some embodiments, the pneumonia comprises a community-related pneumonia. In some embodiments, the pneumonia comprises an aspiration pneumonia. In some embodiments, the pneumonia is a hospital-acquired pneumonia. In some embodiments, the pneumonia is a ventilator-associated pneumonia (VAP) pneumonia. In some embodiments, the hospital-acquired pneumonia comprises VAP. In some embodiments, the VAP is acquired by a patient has been hospitalized and/or intubated. In some embodiments, the ALI is ventilator-induced. In some embodiments, the ventilator induced ALI is associated with increased endothelial permeability. In some embodiments, the ALI or the ARDS is caused by sepsis, trauma, inhalation of a toxic substance, a transfusion, cocaine or other drug overdose, pancreatitis, or a burn.

In some embodiments, the pneumonia comprises an infection-related pneumonia. In some embodiments, the ALI or ARDS comprises an infection-related pneumonia. In some embodiments, the pneumonia does not include an infection-related pneumonia. In some embodiments, the infection-related pneumonia is a hospital-acquired pneumonia.

In some embodiments, the pneumonia comprises a viral pneumonia. In some embodiments, the ALI or ARDS comprises a viral pneumonia. In some embodiments, the infection-related pneumonia does not include a viral pneumonia.

In some embodiments, the viral pneumonia comprises a coronavirus pneumonia, an influenza pneumonia, a rhinovirus pneumonia, an adenovirus pneumonia, or a respiratory syncytial virus pneumonia. In some embodiments, the viral pneumonia comprises a rhinovirus pneumonia. In some embodiments, the viral pneumonia comprises an adenovirus pneumonia. In some embodiments, the viral pneumonia comprises a respiratory syncytial virus pneumonia.

In some embodiments, the viral pneumonia comprises an influenza pneumonia. In some embodiments, the influenza pneumonia comprises an influenza type A pneumonia. In some embodiments, the influenza pneumonia comprises an influenza type B pneumonia.

In some embodiments, the infection-related pneumonia comprises a bacterial pneumonia. In some embodiments, the bacterial pneumonia comprises a Streptococcus pneumonia, a Staphylococcus aureus pneumonia, a Haemophilus influenza pneumonia, a Legionella pneumophilia pneumonia, or a Methicillin resistant Staphylococcus aureus (MRSA) pneumonia. In some embodiments, the bacterial pneumonia comprises a Streptococcus pneumonia. In some embodiments, the bacterial pneumonia comprises a Staphylococcus aureus pneumonia. In some embodiments, the bacterial pneumonia comprises a Haemophilus influenza pneumonia. In some embodiments, the bacterial pneumonia comprises a Legionella pneumophilia pneumonia. In some embodiments, the bacterial pneumonia comprises a Methicillin resistant Staphylococcus aureus (MRSA) pneumonia. In some embodiments, the bacterial pneumonia comprises an atypical pneumonia. An example of an atypical pneumonia is a pneumonia that does not respond to a normal antibiotic. In some embodiments, the atypical pneumonia comprises a Legionella pneumophila. In some embodiments, the atypical pneumonia comprises a Chlamydophila pneumonia.

In some embodiments, the pneumonia comprises a parasite-related pneumonia. In some embodiments, the infection-related pneumonia comprises a Mycoplasma pneumonia. In some embodiments, the infection-related pneumonia comprises a fungal pneumonia. In some embodiments, the fungal pneumonia comprises a Pneumocystis Jirovecii Pneumonia.

In some embodiments, the viral pneumonia comprises a coronavirus pneumonia. In some embodiments, the ALI or ARDS comprises a coronavirus pneumonia. In some embodiments, the viral pneumonia does not include a coronavirus pneumonia. In some embodiments, the coronavirus pneumonia comprises a COVID-19 pneumonia. In some embodiments, the ALI or ARDS comprises a COVID-19 pneumonia. In some embodiments, the coronavirus pneumonia does not include a COVID-19 pneumonia. In some embodiments, the coronavirus pneumonia comprises a severe acute respiratory syndrome (SARS) pneumonia. In some embodiments, the coronavirus pneumonia comprises a Middle East respiratory syndrome (MERS) pneumonia.

There is a strong rationale for treating severe COVID-19 pneumonia with a CRAC inhibitor. Cytokines may drive lung injury in COVID-19 patients. For example, IL-6 may play a role in driving the overactive inflammatory response in the lungs of patients who have severe COVID-19 pneumonia. Elevated IL-2, IL-17 and TNFα may also play a role in severe COVID-19 pneumonia.

CRAC channel inhibitors may have multiple MOAs beneficial to treatment of lung injury. For example, they may inhibit the release of multiple key cytokines: IL-2, IL-6, IL-17, TNFα. They may inhibit a respiratory burst by neutrophils and neutrophil infiltration. They may prevent activation of the pulmonary endothelium and disruption of the alveolar-capillary barrier.

Preclinical data supports the use of CRAC inhibitors (e.g. N-(5-(6-Chloro-2,2-difluorobenzo[d][1,3]dioxol-5-yl)pyrazin-2-yl)-2-fluoro-6-methylbenzamide, Compound 1) in severe COVID-19 pneumonia. In vitro studies in human lymphocytes show Compound 1 inhibits release of multiple cytokines. Animal models of acute pancreatitis (AP) and lung injury show Compound 1 and other CRAC channel inhibitors inhibit cytokines and neutrophil infiltration in lung tissue.

Clinical data supports the use of CRAC inhibitors in severe COVID-19 pneumonia. An injectable emulsion of Compound 1 was safe in healthy volunteers for 365 days, and 90 days in patients with AP and accompanying SIRS plus hypoxemia. Treatment with Compound 1 resulted in a marked reduction in IL-6 levels in patients, and had beneficial effects on respiratory dysfunction in patients.

In some embodiments, the COVID-19 pneumonia comprises a severe pneumonia, or a severe COVID-19 pneumonia. In some embodiments, the ALI or ARDS comprises a severe COVID-19 pneumonia. In some embodiments, the COVID-19 pneumonia comprises a severe or critical COVID-19 pneumonia. In some embodiments, the COVID-19 pneumonia comprises a critical pneumonia, or a critical COVID-19 pneumonia. In some embodiments, the ALI or ARDS comprises a critical COVID-19 pneumonia. In some embodiments, the COVID-19 pneumonia does not include a severe COVID-19 pneumonia. In some embodiments, the COVID-19 pneumonia does not include a critical COVID-19 pneumonia.

In some embodiments, the pneumonia comprises a severe or critical pneumonia. In some embodiments, the ALI or ARDS comprises a severe pneumonia. An example of a severe pneumonia includes a pneumonia with impairment of air exchange or respiratory function. An example of a severe pneumonia includes need for oxygen support such as low-flow oxygen. In some embodiments, the ALI or ARDS comprises a critical pneumonia. An example of a critical pneumonia includes a pneumonia with respiratory failure. An example of a critical pneumonia includes need for additional oxygen support, or a need for high-flow oxygen. An example of a critical pneumonia includes need for mechanical ventilation, or a need for intubation.

### Therapeutic Treatment of Respiratory Disorders Such as ALI/ARDS

Disclosed herein, in some embodiments, are methods of administering a composition described herein to a subject. Some embodiments relate to use a composition described herein, such as administering the composition to a subject.

Some embodiments relate to a method of treating a disorder in a subject in need thereof. Some embodiments relate to use of a composition described herein in the method of treatment. Some embodiments include administering a composition described herein to a subject with the disorder. In some embodiments, the administration treats the disorder in the subject. In some embodiments, the composition treats the disorder in the subject.

In some embodiments, the treatment comprises prevention, inhibition, or reversion of the disorder in the subject. Some embodiments relate to use of a composition described herein in the method of preventing, inhibiting, or reversing the disorder. Some embodiments relate to a method of preventing, inhibiting, or reversing a disorder a disorder in a subject in need thereof. Some embodiments include administering a composition described herein to a subject with the disorder. In some embodiments, the administration prevents, inhibits, or reverses the disorder in the subject. In some embodiments, the composition prevents, inhibits, or reverses the disorder in the subject.

Disclosed herein, are methods for treating or preventing a respiratory disorder. Some embodiments include treating a respiratory disorder. Some embodiments include preventing a respiratory disorder. Some embodiments include treating or alleviating a symptom of a respiratory disorder. Some embodiments include administering to a subject in need thereof, a composition described herein such as a pharmaceutical composition (e.g., a pharmaceutical composition comprising a Calcium channel inhibitor such as a CRAC inhibitor). Some embodiments include identifying the subject in need as having or being at risk of having the respiratory disorder.

In some embodiments, the respiratory disorder comprises an inflammatory disorder. In some embodiments, the inflammatory disorder comprises an ALI or ARDS. In some embodiments, the respiratory disorder comprises an ALI. In some embodiments, the respiratory disorder comprises an ARDS. In some embodiments, the ALI or the ARDS comprises a pneumonia. In some embodiments, the respiratory disorder comprises pneumonia. The pneumonia may, in some cases, be any pneumonia described herein. In some embodiments, the respiratory disorder is hospital-acquired. In some embodiments, the respiratory disorder is ventilator-associated or ventilator-induced.

In some embodiments, the respiratory disorder comprises an infection. In some embodiments, the infection comprises a viral infection. In some embodiments, the viral infection comprises a coronavirus infection. In some embodiments, the coronavirus infection comprises COVID-19. In some embodiments, the coronavirus infection comprises severe acute respiratory syndrome (SARS). In some embodiments, the infection comprises Middle East respiratory syndrome (MERS). In some embodiments, the viral infection comprises a rhinovirus infection. In some embodiments, the viral infection comprises an adenovirus infection. In some embodiments, the viral infection comprises a respiratory syncytial virus infection. In some embodiments, the viral infection comprises an influenza infection. In some embodiments, the influenza comprises influenza type A. In some embodiments, the influenza comprises influenza type B. In some embodiments, the influenza comprises influenza type C. In some embodiments, the influenza comprises influenza type D. In some embodiments, the influenza comprises a hemagglutinin subtype such as H1, H2, H3, H5, H6, H7, H9, or H10. In some embodiments, the influenza comprises a neuraminidase subtype such as N1, N2, N6, N7, N8, or N9.

In some embodiments, the infection comprises a bacterial infection. In some embodiments, the bacterial infection comprises a Streptococcus infection. In some embodiments, the bacterial infection comprises a Staphylococcus aureus infection. In some embodiments, the bacterial infection comprises a Haemophilus influenza infection. In some embodiments, the bacterial infection comprises a Legionella pneumophilia infection. In some embodiments, the bacterial infection comprises a Methicillin resistant Staphylococcus aureus (MRSA) infection. In some embodiments, the bacterial infection comprises a Legionella pneumophila. In some embodiments, the bacterial infection comprises a Chlamydophila infection.

In some embodiments, the infection comprises a parasite-related infection. In some embodiments, the infection-related infection comprises a Mycoplasma infection. In some embodiments, the infection-related infection comprises a fungal infection. In some embodiments, the fungal infection comprises a Pneumocystis Jirovecii Infection.

Disclosed herein are compositions and methods for treating acute lung injury (ALI) or acute respiratory distress syndrome (ARDS) in a subject comprising administering a therapeutically effective amount of an intracellular Calcium signaling inhibitor to said subject. Further, disclosed herein are compositions and methods for preventing acute lung injury (ALI) or acute respiratory distress syndrome (ARDS) in a subject at risk of developing ALI or ARDS, comprising administering a therapeutically effective amount of an intracellular Calcium signaling inhibitor to said subject.

The compositions and methods may be used in some embodiments for treating any ALI or ARDS, including, but not limited to a pneumonia, a viral pneumonia, a coronavirus pneumonia, a COVID-19 pneumonia, a severe COVID-19 pneumonia such as a COVID-19 with impairment of air exchange or respiratory function, and/or a critical COVID-19 pneumonia such as a COVID-19 with respiratory failure. The compositions and methods may be used for treating an infection. The compositions and methods may be used for treating a viral infection. The compositions and methods may be used for treating a coronavirus infection. The compositions and methods may be used for treating COVID-19. In some embodiments, a composition or method described herein includes a measure to treat pneumonia, such as a pneumonia described herein. The treatment may comprise administration of a compound or composition described herein to a subject. The subject may be identified as having a disease or disorder disclosed herein. The subject may be identified as being at risk of having a disease or disorder disclosed herein.

The compositions and methods may be used in some embodiments for preventing any ALI or ARDS, including, but not limited to a pneumonia, a viral pneumonia, a coronavirus pneumonia, a COVID-19 pneumonia, a severe COVID-19 pneumonia such as a COVID-19 with impairment of air exchange or respiratory function, and/or a critical COVID-19 pneumonia such as a COVID-19 with respiratory failure. The compositions and methods may be used for preventing an infection. The compositions and methods may be used for preventing a viral infection. The compositions and methods may be used for preventing a coronavirus infection. The compositions and methods may be used for preventing COVID-19. In some embodiments, a composition or method described herein includes a measure to prevent pneumonia, such as a pneumonia described herein. The prevention may comprise administration of a compound or composition described herein to a subject. The subject may be identified as having a disease or disorder disclosed herein. The subject may be identified as being at risk of having a disease or disorder disclosed herein.

In some embodiments, the ALI or ARDS comprises pneumonia. In some embodiments, the pneumonia comprises a severe pneumonia. In some embodiments, the pneumonia comprises a critical pneumonia. In some embodiments, the pneumonia comprises a viral pneumonia. In some embodiments, the viral pneumonia comprises a viral pneumonia due to a coronavirus, an adenovirus, an influenza virus, a rhinovirus, or a respiratory syncytial virus. In some embodiments, the viral pneumonia comprises a viral pneumonia due to a coronavirus. In some embodiments, the coronavirus is SARS-CoV, SARS-CoV-2, or MERS-CoV. In some embodiments, the coronavirus is SARS-CoV-2. In some embodiments, the pneumonia comprises a severe or critical COVID-19 pneumonia.

The methods of the present invention can further comprise identifying the subject as having a respiratory failure or disease. In some embodiments, the method further comprises identifying the subject as having ALI and/or ARDS. In some embodiments, the method further comprises identifying the subject as having the ALI. In some embodiments, the method further comprises identifying the subject as having the ARDS.

In some embodiments, the intracellular Calcium signaling inhibitor is delivered to achieve a tissue level concentration that is equal to, about, or greater than the in vitro IC₅₀ value determined for the compound. In some embodiments the Calcium signaling inhibitor is delivered to achieve a tissue level concentration that is 1.5×. 2×, 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50×, 51×, 52×, 53×, 54×, 55×, 56×, 57×, 58×, 59×, 60×, 61×, 62×, 63×, 64×, 65×, 66×, 67×, 68×, 69×, 70×, 71×, 72×, 73×, 74×, 75×, 76×, 77×, 78×, 79×, 80×, 81×, 82×, 83×, 84×, 85×, 86×, 87×, 88×, 89×, 90×, 91×, 92×, 93×, 94×, 95×, 96×, 97×, 98×, 99×, 100×, or any non-integer multiple ranging from 1× to 100× of the in vitro IC₅₀ value determined for the compound.

In some embodiments the Calcium signaling inhibitor is delivered to achieve a tissue level concentration that ranges from 1× to 100×, 2× to 80×, 3× to 60×, 4× to 50×, 5× to 45×, 6× to 44×, 7× to 43×, 8× to 43×, 9× to 41×, or 10× to 40×, or any non-integer within said range, of the in vitro IC₅₀ value determined for the compound.

In some embodiments the Calcium signaling inhibitor is delivered to achieve a tissue level concentration that is 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, 50 μM, 51 μM, 52 μM, 53 μM, 54 μM, 55 μM, 56 μM, 57 μM, 58 μM, 59 μM, 60 μM, 61 μM, 62 μM, 63 μM, 64 μM, 65 μM, 66 μM, 67 μM, 68 μM, 69 μM, 70 μM, 71 μM, 72 μM, 73 μM, 74 μM, 75 μM, 76 μM, 77 μM, 78 μM, 79 μM, 80 μM, 81 μM, 82 μM, 83 μM, 84 μM, 85 μM, 86 μM, 87 μM, 88 μM, 89 μM, 90 μM, 91 μM, 92 μM, 93 μM, 94 μM, 95 μM, 96 μM, 97 μM, 98 μM, 99 μM, 100 μM, or any non-integer multiple ranging from about 1 μM to about 100 μM.

In some embodiments the Calcium signaling inhibitor is delivered to achieve a tissue level concentration that ranges from 1 μM to 100 μM, 2 μM to 90 μM, 3 μM to 80 μM, 4 μM to 70 μM, 5 μM to 60 μM, 6 μM to 50 μM, 7 μM to 40 μM, 8 μM to 30 μM, 9 μM to 20 μM, or 10 μM to 40 μM, or any integer or non-integer within said range.

In some embodiments the Calcium signaling inhibitor is delivered to achieve a tissue level concentration that ranges from 9.5 μM to 10.5 μM, 9 μM to 11 μM, 8 μM to 12 μM, 7 μM to 13 μM, 5 μM to 15 μM, 2 μM to 20 μM or 1 μM to 50 μM, or any integer or non-integer within said range.

In one embodiment is a method for treating a patient having cytokine storm syndrome comprising administering to the patient in need a therapeutically effective amount of N-(5-(6-Chloro-2,2-difluorobenzo[d][1,3]dioxol-5-yl)pyrazin-2-yl)-2-fluoro-6-methylbenzamide. In another embodiment, the patient is administered N-(5-(6-Chloro-2,2-difluorobenzo[d][1,3]dioxol-5-yl)pyrazin-2-yl)-2-fluoro-6-methylbenzamide via intravenously. In a further embodiment is a method for inhibiting the release of multiple key cytokines comprising administering to the patient a therapeutically effective amount of N-(5-(6-Chloro-2,2-difluorobenzo[d][1,3]dioxol-5-yl)pyrazin-2-yl)-2-fluoro-6-methylbenzamide. In a further embodiment is a method for inhibiting release of IL-2, IL-6, IL-17, and/or TNFα comprising administering an effective amount of N-(5-(6-Chloro-2,2-difluorobenzo[d][1,3]dioxol-5-yl)pyrazin-2-yl)-2-fluoro-6-methylbenzamide. In yet another embodiment is a method of inhibiting excessive or uncontrolled release of proinflammatory cytokines comprising administering an effective amount of N-(5-(6-Chloro-2,2-difluorobenzo[d][1,3]dioxol-5-yl)pyrazin-2-yl)-2-fluoro-6-methylbenzamide.

N-(5-(6-Chloro-2,2-difluorobenzo[d][1,3]dioxol-5-yl)pyrazin-2-yl)-2-fluoro-6-methylbenzamide (Compound 1) is an example of a composition described herein, and inhibits calcium release-activated calcium (CRAC) channels. A pharmaceutical composition comprising Compound 1 has been demonstrated to be safe and potentially efficacious in critically ill patients with acute pancreatitis. Its rapid onset may be beneficial for acute settings. It may prevent the development of ARDS in patients with severe COVID-19 pneumonia and/or reduce the need for ventilators.

Compound 1 is a potent and selective small molecule inhibitor of CRAC channels. CRAC channels are found on many cell types, including immune cells, where aberrant activation of these channels may play a key role in the pathobiology of acute and chronic inflammatory syndromes.

Due to the fast-acting nature of Compound 1, it and other CRAC channel inhibitors may quickly lessen the cytokine storm associated with COVID-19 and may stabilize the pulmonary endothelial capillary barrier and prevent more serious lung injury. For patients infected with SARS-CoV-2, morbidity and mortality can arise from host immune responses. These responses can lead to cytokine storm, which in turn causes severe pneumonia and hypoxemic respiratory failure, ARDS, death or in the case of survivors, permanently compromised pulmonary function.

Some embodiments of the methods described herein include obtaining a baseline measurement from a subject. For example, in some embodiments, a baseline measurement is obtained from the subject prior to treating the subject. Examples of baseline measurements include a baseline protein measurement, a baseline mRNA measurement, a baseline lung inflammation measurement, a baseline lung myeloperoxidase activity (e.g., neutrophil infiltration) measurement, a baseline cytokine measurement (e.g. protein or mRNA levels of a cytokine such as TNFα, IL-2, IL-6, IL-17, IFN-α, IFN-β, IFN-ω, and IFN-γ), a baseline cytokine panel measurement, a baseline procalcitonin measurement, a baseline measurement of persistent systemic inflammatory response syndrome, a baseline procalcitonin measurement, a baseline endothelial cell Ca²⁺ flux measurement, a baseline lung injury measurement, a baseline endothelial lung dysfunction measurement, a baseline respiratory failure measurement (e.g. severity or duration), a baseline need for supplemental oxygen or ventilatory support, a baseline measurement of an amount or duration of supplemental oxygen or ventilatory support, a baseline lung fluid measurement, a baseline PaO₂ measurement, a baseline FiO₂ measurement, a baseline PaO₂/FiO₂ measurement, a baseline SaO₂ measurement, a baseline ordinal scale measurement, a baseline time to hospital discharge measurement, a baseline body temperature measurement, a baseline fever measurement, or a baseline heart rate measurement.

In some embodiments, the baseline measurement is obtained by performing an assay such as an immunoassay, a colorimetric assay, or a fluorescence assay, on the sample obtained from the subject. In some embodiments, the baseline measurement is obtained by an immunoassay, a colorimetric assay, or a fluorescence assay. In some embodiments, the baseline measurement is obtained by PCR. In some embodiments, the PCR comprises RT-qPCR or RT-qPCR. For example, quantitation or confirmation of viral particles such as SARS-Cov-2 nucleic acids may be obtained using an RT-PCR assay of a nasal swab, pharyngeal swab, or respiratory tract aspirate.

In some embodiments, the baseline measurement is obtained directly in or on the subject. In some embodiments, the baseline measurement is obtained with a nasal cannula. In some embodiments, the baseline measurement is obtained with pulse oximetry. In some embodiments, the baseline measurement is obtained with a thermometer. In some embodiments, the baseline measurement is obtained by making a visual inspection of the subject. In some embodiments, the baseline measurement is obtained with a medical imaging device.

Some embodiments of the methods described herein include obtaining a sample from a subject. In some embodiments, the baseline measurement is obtained from the subject prior to administration of a composition described herein. In some embodiments, the baseline measurement is obtained in a sample obtained from the subject. In some embodiments, the sample is obtained from the subject prior to administration or treatment of the subject with a composition described herein. In some embodiments, a baseline measurement is obtained in a sample obtained from the subject prior to administering the composition to the subject.

In some embodiments, the sample comprises a fluid. In some embodiments, the sample is a fluid sample. In some embodiments, the fluid sample is bronchoalveolar lavage fluid (BAL) sample. In some embodiments, the sample comprises a nasal sample. In some embodiments, the sample comprises a pharyngeal sample. In some embodiments, the sample comprises a swab (e.g., a nasal swab or a pharyngeal swab). In some embodiments, the sample comprises an aspirate. In some embodiments, the sample comprises a respiratory tract sample (e.g., a respiratory tract aspirate). In some embodiments, the sample comprises or consists of a blood, plasma, or serum sample. In some embodiments, the sample is a blood sample. In some embodiments, the sample is a plasma sample. In some embodiments, the sample is a serum sample. In some embodiments, the sample comprises a tissue. In some embodiments, the sample is a tissue sample. In some embodiments, the sample comprises or consists of lung tissue. In some embodiments, the sample comprises or consists of one or more lung cells. The lung cells may be epithelial cells or endothelial cells. In some embodiments, the sample comprises or consists of one or more endothelial cells such as pulmonary endothelial cells. In some embodiments, the sample comprises or consists of one or more epithelial cells such as alveolar epithelial cells.

In some embodiments, the composition or administration of the composition affects a measurement such as a protein measurement, a mRNA measurement, a lung inflammation measurement, a lung myeloperoxidase activity (e.g., neutrophil infiltration) measurement, a cytokine measurement (e.g. protein or mRNA levels of a cytokine such as TNFα, IL-6, IL-17, other cytokines), a cytokine panel measurement, a procalcitonin measurement, a measurement of persistent systemic inflammatory response syndrome, a procalcitonin measurement, a endothelial cell Ca²⁺ flux measurement, a lung injury measurement, a endothelial lung dysfunction measurement, a respiratory failure measurement (e.g. severity or duration), a need for supplemental oxygen or ventilatory support, a measurement of an amount or duration of supplemental oxygen or ventilatory support, a lung fluid measurement, a PaO₂ measurement, a FiO₂ measurement, a PaO₂/FiO₂ measurement, a SaO₂ measurement, a time to hospital discharge measurement, an ordinal scale measurement, a body temperature measurement, a fever measurement, or a heart rate measurement. In some embodiments, the composition improves the measurement relative to the baseline measurement. In some embodiments, another measure is improved, such as a symptom or marker associated with the disorder (for example, as discussed in the section on Acute lung injury (ALI) and its severe manifestation, acute respiratory distress syndrome (ARDS)s). In some embodiments, the composition reduces the measurement relative to the baseline measurement. In some embodiments, the composition increases the measurement relative to the baseline measurement.

Some embodiments of the methods described herein include obtaining the measurement from a subject. For example, the measurement may be obtained from the subject after treating the subject. In some embodiments, the CRAC inhibitor is administered to a subject at 0 hour (start of the first infusion of CRAC inhibitor) with an initial dose and a subsequent different dose may be administered at 24 hours and 48 hours after 0 hour. In some embodiments, the subsequence does of the CRAC inhibitor may be administered 72 hours after 0 hour. In some embodiments, the measurement is obtained in samples collected at aforementioned 24 hours. In some embodiments, the measurement is obtained in samples collected at aforementioned 48 hours. In some embodiments, the measurement is obtained in samples collected at aforementioned 72 hours. In some embodiments, the measurement is obtained in a second sample described herein (such as a blood, plasma, serum, or lung sample) obtained from the subject after the composition is administered to the subject. In some embodiments, the measurement is an indication that the disorder has been treated. In some embodiments, the measurement is obtained directly from the subject. In some embodiments, the measurement is obtained noninvasively, such as by using an imaging device.

In some embodiments, following administration of the composition (e.g. an intracellular Calcium signaling inhibitor, or a CRAC inhibitor), the subject has an improvement (e.g. an increase in numerical value) in an ordinal scale comprising an ordinal pneumonia scale such as the following: 1. Death 2. Hospitalized, on invasive mechanical ventilation or ECMO 3. Hospitalized, on non-invasive ventilation or high flow oxygen devices 4. Hospitalized, requiring low flow supplemental oxygen 5. Hospitalized, not requiring supplemental oxygen-requiring ongoing medical care (coronavirus (e.g. COVID-19) related or otherwise) 6. Hospitalized, not requiring supplemental oxygen-no longer requires ongoing medical care (other than per protocol study drug administration) 7. Not hospitalized.

In some embodiments, administration of the composition (e.g. an intracellular Calcium signaling inhibitor, or a CRAC inhibitor) to a subject reduces a hospital time measurement for the subject. In some embodiments, the hospital time measurement is an amount of time in a hospital. In some embodiments, the hospital time measurement is a time to hospital discharge measurement. In some embodiments, administration of the composition reduces the number of hospitalizations.

In some embodiments, administration of the composition (e.g. an intracellular Calcium signaling inhibitor, or a CRAC inhibitor) to a subject improves a partial pressure of oxygen (PaO₂) value in the subject. For example, the PaO₂ value may be increased by the administration of the composition. In some embodiments, administration of the composition to a subject improves a partial pressure of oxygen to fraction of inspired oxygen ratio (PaO₂/FiO₂) in the subject. For example, the PaO₂/FiO₂ may be increased by the administration of the composition.

In some embodiments, administration of the composition to a subject prevents, reduces or eliminates a need for supplemental oxygen or ventilatory support for the subject. In some embodiments, administration of the composition prevents a need for supplemental oxygen support. In some embodiments, administration of the composition reduces a need for supplemental oxygen support. In some embodiments, administration of the composition eliminates a need for supplemental oxygen support. In some embodiments, administration of the composition prevents a need for supplemental ventilatory support. In some embodiments, administration of the composition reduces a need for supplemental ventilatory support. In some embodiments, administration of the composition eliminates a need for supplemental ventilatory support. In some embodiments, administration of the composition reduces an amount of supplemental oxygen support. In some embodiments, administration of the composition reduces a duration of supplemental oxygen support. In some embodiments, administration of the composition reduces an amount of supplemental ventilatory support. In some embodiments, administration of the composition reduces a duration of supplemental ventilatory support.

Disclosed herein, in some embodiments are methods of treatment that may include administration of a compound disclosed herein to a subject. Some embodiments further comprise administering a respiratory treatment to the subject. In some embodiments, the respiratory treatment comprises respiratory assistance. In some embodiments, the respiratory assistance comprises intubation such as endotracheal intubation, ventilation such as mechanical ventilation or noninvasive ventilation, or oxygen support. In some embodiments, the subject has already been administered a respiratory treatment such as a respiratory assistance prior to administration of the composition, or prior to initiation of a treatment with the composition. In some embodiments, the respiratory treatment and the composition are administered concurrently. In some embodiments, the respiratory treatment and the treatment with the composition overlap.

In some embodiments, the Calcium channel inhibitor (e.g., Auxora) is administered at a dosage of about 0.5 mg/kg, 1.0 mg/kg, 1.5 mg/kg, 2 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 125 mg/kg, 150 mg/kg, 175 mg/kg, 200 mg/kg, 225 mg/kg, 250 mg/kg, or any numbers between any two forgoing values.

Combination Administration with a Compound for Treating ALI/ARDS

Disclosed herein are compositions and administration regimens for the combinatorial administration of a Calcium channel inhibitor and a at least a compound for treating ALI or ARDS. In some embodiments an administration regimen comprises administration to a subject of a compound for treating ALI or ARDS, and administration of an intracellular Calcium signaling inhibitor. In some embodiments, an administration regimen comprises administration to a subject a corticosteroid or immunosuppressive drug and an intracellular Calcium signaling inhibitor. In some embodiments, the therapeutically effective amount of the corticosteroid and/or immunosuppressive drug and the intracellular Calcium signaling inhibitor is administered at different times. In some embodiments, wherein the therapeutically effective amount of the corticosteroid and/or immunosuppressive drug and the intracellular Calcium signaling inhibitor is administered concurrently.

In some embodiments, the corticosteroid is a glucocorticoid or a mineralocorticoid. In some embodiments, the corticosteroid is aldosterone, corticosterone, cortisol, cortisone, pregnenolone, progesterone, flugestone, fluorometholone, medrysone, prebedilone acetate, chloroprednisone, cloprednol, difluprednate, fludrocorisone, fluocinolone, fluoperolone, loteprednol, methylprednisolone, prednicarbate, prednisolone, prednisone, tixocortol, triamcinolone, alclometasone, beclomethasone, betamethasone, clobetasol, clobetasone, clocortolone, desoximetasone, dexamethasone, diflorasone, difluocortolone, fluclorolone, flumetasone, fluocortin, fluocortolone, fluprednidene, fluticasone, fluticasone furoate, halometasone, medprednisone, mometasone, mometasone furoate, paramethasone, prednylidene, rimexolone, ulobetasol, amcinonide, budesonide, ciclesonide, deflazacort, desonide, formocortal, fluclorolone acetonide, fludroxycortide, flunisolide, fluocinolone acetonide, fluocinonide, halcinonide, or triamcinolone acetonide. In some embodiments, the corticosteroid is cortisol, cortisone, prednisone, prednisolone, methylprednisolone, dexamethasone, betamethasone, triamcinolone, fludrocortisone, fludrocortisone acetate, deoxycorticosterone, deoxycorticosterone acetate, aldosterone, beclomethasone, or progesterone.

In some embodiments, the immunosuppressive drug is not cyclosporine, tacrolimus, sirolimus, everolimus, azathioprine, cyclosphosphamide, methotrexate, mycophenolate, leflunomide, abatacept, adalimumab, alemtuzumab, anakinra, basilizimab, belimumab, bevacizumab, brodalumab, canakinumab, certolizumab, cetuximab, clazakizumab, daclizumab, eculizumab, etanercept, golimumab, infliximab, interferon, ixekizumab, muromonab, natalizumab, omalizumab, rituximab, sarilumab, secukinumab, trastuzumab, ustekinumab, vedolizumab, baricitinib, tofacitinib, or other janus kinase inhibitors. In some embodiments, the immunosuppressive drug is corticosteroid, a glucocorticoid, an anti-IL-6, an immunomodulatory imide drug, 4-deoxypyridoxine, fingolimod, laquinimod, mitzoribine, mycophenolic acid, pimecrolimus, tocilizumab or voclosporin. In some embodiments, the immunosuppressive drug is tocilizumab.

In some embodiments the compound is selected from the list consisting of a prostaglandin inhibitor, complement inhibitor, β-agonist, beta-2 agonist, granulocyte macrophage colony-stimulating factor, corticosteroid, N-acetylcysteine, statin, glucagon-like peptide-1 (7-36) amide (GLP-1), triggering receptor expressed on myeloid cells (TREM1) blocking peptide, 17-allylamino-17-demethoxygeldanamycin (17-AAG), antibody to tumor necrosis factor (TNF), recombinant interleukin (IL)-1 receptor antagonist, cisatracurium besilate, and Angiotensin-Converting Enzyme (ACE) Inhibitor. In some embodiments, the compound includes an antiviral compound. In some embodiments, the antiviral compound is an anti-coronavirus compound. In some embodiments, the anti-coronavirus compound comprises remdesivir. Examples of antiviral compounds include antiretroviral compounds, protease inhibitors, nucleoside reverse transcriptase inhibitors, reverse transcriptase inhibitors, integrase inhibitors, entry inhibitors, maturation inhibitors, anti-influenza compounds, peramivir, zanamivir, oseltamivir, baloxavir marboxil, and pharmaceutically acceptable salts thereof. In some embodiments, the compound comprises an antibiotic. In some embodiments, the compound comprises an anti-malarial drug. In some embodiments, the compound comprises hydroxychloroquine. In some embodiments, the compound comprises chloroquine.

In some embodiments the intracellular Calcium signaling inhibitor is an SOC inhibitor. In some embodiments the intracellular Calcium signaling inhibitor is a CRAC inhibitor. An exemplary CRAC inhibitor comprises N-(5-(6-Chloro-2,2-difluorobenzo[d][1,3]dioxol-5-yl)pyrazin-2-yl)-2-fluoro-6-methylbenzamide, having a structure of.

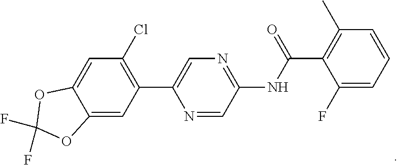 [SMILES: Cc1cccc(F)c1C(=O)Nc1cnc(-c2cc3c(cc2Cl)OC(F)(F)O3)cn1]

An exemplary CRAC inhibitor comprises GSK-7975A. An exemplary CRAC inhibitor comprises BTP2. An exemplary CRAC inhibitor comprises 2,6-Difluoro-N-(1-(4-hydroxy-2-(trifluoromethyl)benzyl)-1H-pyrazol-3-yl)benzamide.

In some embodiments the administration regimen comprises administration of a calcium channel inhibitor such as a CRAC inhibitor such as at least one of N-(5-(6-Chloro-2,2-difluorobenzo[d][1,3]dioxol-5-yl)pyrazin-2-yl)-2-fluoro-6-methylbenzamide and BTP2, and a compound for treating ALI/ARDS. In some embodiments the calcium channel inhibitor such as a CRAC inhibitor such as at least one of N-(5-(6-Chloro-2,2-difluorobenzo[d][1,3]dioxol-5-yl)pyrazin-2-yl)-2-fluoro-6-methylbenzamide and BTP2 is administered on the same day as a compound for treating ALI/ARDS on lung activities. In some embodiments the calcium channel inhibitor such as a CRAC inhibitor such as at least one of N-(5-(6-Chloro-2,2-difluorobenzo[d][1,3]dioxol-5-yl)pyrazin-2-yl)-2-fluoro-6-methylbenzamide and BTP2 is administered on the same week as a compound for treating ALI/ARDS. In some embodiments the calcium channel inhibitor such as a CRAC inhibitor such as at least one of N-(5-(6-Chloro-2,2-difluorobenzo[d][1,3]dioxol-5-yl)pyrazin-2-yl)-2-fluoro-6-methylbenzamide and BTP2 is administered concurrently with each administration of a compound for treating ALI/ARDS. In some embodiments the calcium channel inhibitor such as a CRAC inhibitor such as at least one of N-(5-(6-Chloro-2,2-difluorobenzo[d][1,3]dioxol-5-yl)pyrazin-2-yl)-2-fluoro-6-methylbenzamide and BTP2 is administered on an administration regimen pattern that is independent of the administration pattern for a compound for treating ALI/ARDS. In some embodiments the calcium channel inhibitor such as a CRAC inhibitor such as at least one of N-(5-(6-Chloro-2,2-difluorobenzo[d][1,3]dioxol-5-yl)pyrazin-2-yl)-2-fluoro-6-methylbenzamide and BTP2 is administered through the same route of delivery, such as orally or intravenously, as a compound for treating ALI/ARDS. In some embodiments the calcium channel inhibitor such as a CRAC inhibitor such as at least one of N-(5-(6-Chloro-2,2-difluorobenzo[d][1,3]dioxol-5-yl)pyrazin-2-yl)-2-fluoro-6-methylbenzamide and BTP2 is administered through a separate route of delivery compared to a compound for treating ALI/ARDS. In some embodiments the calcium channel inhibitor such as a CRAC inhibitor such as at least one of N-(5-(6-Chloro-2,2-difluorobenzo[d][1,3]dioxol-5-yl)pyrazin-2-yl)-2-fluoro-6-methylbenzamide and BTP2 is administered to a person receiving a compound for treating ALI/ARDS only after said person shows at least one sign of an impact of said drug on lung activity. In some embodiments the calcium channel inhibitor such as a CRAC inhibitor such as at least one of N-(5-(6-Chloro-2,2-difluorobenzo[d][1,3]dioxol-5-yl)pyrazin-2-yl)-2-fluoro-6-methylbenzamide and BTP2 is administered to a person receiving a compound for treating ALI/ARDS in the absence of any evidence in or from said person related to any sign of an impact of said compound on lung activity.

In some embodiments the calcium channel inhibitor such as a CRAC inhibitor such as at least one of N-(5-(6-Chloro-2,2-difluorobenzo[d][1,3]dioxol-5-yl)pyrazin-2-yl)-2-fluoro-6-methylbenzamide and BTP2 is administered in a single composition with a compound for treating ALI/ARDS. Accordingly, some embodiments disclosed herein relate to a composition comprising an intracellular Calcium signaling inhibitor and at least one compound for treating ALI/ARDS. In some embodiments the at least one drug selected from the list consisting of: a prostaglandin inhibitor, complement inhibitor, β-agonist, beta-2 agonist, granulocyte macrophage colony-stimulating factor, corticosteroid, N-acetylcysteine, statin, glucagon-like peptide-1 (7-36) amide (GLP-1), triggering receptor expressed on myeloid cells (TREM1) blocking peptide, 17-allylamino-17-demethoxygeldanamycin (17-AAG), antibody to tumor necrosis factor (TNF), recombinant interleukin (IL)-1 receptor antagonist, cisatracurium besilate, and Angiotensin-Converting Enzyme (ACE) Inhibitor.

In some embodiments, the intracellular Calcium signaling inhibitor is delivered to achieve a tissue level concentration that is equal to, about, or greater than the in vitro IC₅₀ value determined for the compound. In some embodiments the Calcium signaling inhibitor is delivered to achieve a tissue level concentration that is 1.5×. 2×, 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50×, 51×, 52×, 53×, 54×, 55×, 56×, 57×, 58×, 59×, 60×, 61×, 62×, 63×, 64×, 65×, 66×, 67×, 68×, 69×, 70×, 71×, 72×, 73×, 74×, 75×, 76×, 77×, 78×, 79×, 80×, 81×, 82×, 83×, 84×, 85×, 86×, 87×, 88×, 89×, 90×, 91×, 92×, 93×, 94×, 95×, 96×, 97×, 98×, 99×, 100×, or any non-integer multiple ranging from 1× to 100× of the in vitro IC₅₀ value determined for the compound.

In some embodiments the Calcium signaling inhibitor is delivered to achieve a tissue level concentration that ranges from 1× to 100×, 2× to 80×, 3× to 60×, 4× to 50×, 5× to 45×, 6× to 44×, 7× to 43×, 8× to 43×, 9× to 41×, or 10× to 40×, or any non-integer within said range, of the in vitro IC₅₀ value determined for the compound.

In some embodiments the Calcium signaling inhibitor is delivered to achieve a tissue level concentration that is 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, 50 μM, 51 μM, 52 μM, 53 μM, 54 μM, 55 μM, 56 μM, 57 μM, 58 μM, 59 μM, 60 μM, 61 μM, 62 μM, 63 μM, 64 μM, 65 μM, 66 μM, 67 μM, 68 μM, 69 μM, 70 μM, 71 μM, 72 μM, 73 μM, 74 μM, 75 μM, 76 μM, 77 μM, 78 μM, 79 μM, 80 μM, 81 μM, 82 μM, 83 μM, 84 μM, 85 μM, 86 μM, 87 μM, 88 μM, 89 μM, 90 μM, 91 μM, 92 μM, 93 μM, 94 μM, 95 μM, 96 μM, 97 μM, 98 μM, 99 μM, 100 μM, or any non-integer multiple ranging from about 1 μM to about 100 μM.

In some embodiments the Calcium signaling inhibitor is delivered to achieve a tissue level concentration that ranges from 1 μM to 100 μM, 2 μM to 90 μM, 3 μM to 80 μM, 4 μM to 70 μM, 5 μM to 60 μM, 6 μM to 50 μM, 7 μM to 40 μM, 8 μM to 30 μM, 9 μM to 20 μM, or 10 μM to 40 μM, or any integer or non-integer within said range.

In some embodiments the Calcium signaling inhibitor is delivered to achieve a tissue level concentration that ranges from 9.5 μM to 10.5 μM, 9 μM to 11 μM, 8 μM to 12 μM, 7 μM to 13 μM, 5 μM to 15 μM, 2 μM to 20 μM or 1 μM to 50 μM, or any integer or non-integer within said range.

### Pharmaceutical Compositions

Provided herein can be pharmaceutical compositions comprising at least one of the Calcium signaling inhibitors described herein. In some cases, the pharmaceutical compositions comprise at least one of the Calcium signaling inhibitors and at least one of the compounds for treating ALI and/or ARDS disclosed herein.

Pharmaceutical compositions provided herein can be introduced as oral forms, transdermal forms, oil formulations, edible foods, food substrates, aqueous dispersions, emulsions, injectable emulsions, solutions, suspensions, elixirs, gels, syrups, aerosols, mists, powders, capsule, tablets, nanoparticles, nanoparticle suspensions, nanoparticle emulsions, lozenges, lotions, pastes, formulated sticks, balms, creams, and/or ointments.

In some embodiments, the pharmaceutical composition additionally comprises at least one of an excipient, a solubilizer, a surfactant, a disintegrant, and a buffer. In some embodiments, the pharmaceutical composition is free of pharmaceutically acceptable excipients. The term “pharmaceutically acceptable excipient”, as used herein, means one or more compatible solid or encapsulating substances, which are suitable for administration to a subject. The term “compatible”, as used herein, means that the components of the composition are capable of being commingled with the subject compound, and with each other, in a manner such that there is no interaction, which would substantially reduce the pharmaceutical efficacy of the composition under ordinary use situations. In some embodiments, the pharmaceutically acceptable excipient is of sufficiently high purity and sufficiently low toxicity to render them suitable for administration preferably to an animal, preferably mammal, being treated.

Some examples of substances, which can serve as pharmaceutically acceptable excipients include: amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. In some embodiments, the amino acid is arginine. In some embodiments, the amino acid is L-arginine; monosaccharides such as glucose (dextrose), arabinose, mannitol, fructose (levulose), and galactose; cellulose and its derivatives such as sodium carboxymethyl cellulose, ethyl cellulose, and methyl cellulose; solid lubricants such as talc, stearic acid, magnesium stearate and sodium stearyl fumarate; polyols such as propylene glycol, glycerin, sorbitol, mannitol, and polyethylene glycol; emulsifiers such as the polysorbates; wetting agents such as sodium lauryl sulfate, Tween®, Span, alkyl sulphates, and alkyl ethoxylate sulphates; cationic surfactants such as cetrimide, benzalkonium chloride, and cetylpyridinium chloride; diluents such as calcium carbonate, microcrystalline cellulose, calcium phosphate, starch, pregelatinized starch, sodium carbonate, mannitol, and lactose; binders such as starches (corn starch and potato starch), gelatin, sucrose hydroxypropyl cellulose (HPC), polyvinylpyrrolidone (PVP), and hydroxypropyl methyl cellulose (HPMC); disintegrants such as starch, and alginic acid; super-disintegrants such as ac-di-sol, croscarmellose sodium, sodium starch glycolate and crospovidone.

Glidants such as silicon dioxide; coloring agents such as the FD&C dyes; sweeteners and flavoring agents, such as aspartame, saccharin, menthol, peppermint, and fruit flavors; preservatives such as benzalkonium chloride, PHMB, chlorobutanol, thimerosal, phenylmercuric, acetate, phenylmercuric nitrate, parabens, and sodium benzoate; tonicity adjustors such as sodium chloride, potassium chloride, mannitol, and glycerin; antioxidants such as sodium bisulfite, acetone sodium bisulfite, sodium formaldehyde, sulfoxylate, thiourea, and EDTA; pH adjuster such as NaOH, sodium carbonate, sodium acetate, HCl, and citric acid; cryoprotectants such as sodium or potassium phosphates, citric acid, tartaric acid, gelatin, and carbohydrates such as dextrose, mannitol, and dextran; surfactants such as sodium lauryl sulfate. For example, cationic surfactants such as cetrimide (including tetradecyl trimethyl ammonium bromide with dodecyl and hexadecyl compounds), benzalkonium chloride, and cetylpyridinium chloride. Some examples of anionic surfactants are alkylsulphates, alkylethoxylate sulphates, soaps, carxylate ions, sulfate ions, and sulfonate ions. Some examples of non-ionic surfactants are polyoxyethylene derivatives, polyoxypropylene derivatives, polyol derivatives, polyol esters, polyoxyethylene esters, poloxamers, glocol, glycerol esters, sorbitan derivatives, polyethylene glycol (such as PEG-40, PEG-50, or PEG-55) and esters of fatty alcohols; organic materials such as carbohydrates, modified carbohydrates, lactose (including a-lactose, monohydrate spray dried lactose or anhydrous lactose), starch, pregelatinized starch, sucrose, mannitol, sorbital, cellulose (including powdered cellulose and microcrystalline cellulose); inorganic materials such as calcium phosphates (including anhydrous dibasic calcium phosphate, dibasic calcium phosphate or tribasic calcium phosphate); co-processed diluents; compression aids; anti-tacking agents such as silicon dioxide and talc.

In some embodiments, the pharmaceutical compositions described herein are provided in unit dosage form. As used herein, a “unit dosage form” is a composition containing an amount of the at least one of the Calcium signaling inhibitors and/or the at least one of the compounds for treating ALI and/or ARDS that is suitable for administration to a subject in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded.

### Certain Terminology

Unless defined otherwise, all technical and scientific terms used herein have the same meaning as is commonly understood to which the claimed subject matter pertains. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described.

Definition of standard chemistry terms may be found in reference works, including but not limited to, Carey and Sundberg “Advanced Organic Chemistry 4th Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology.

Unless specific definitions are provided, the nomenclature employed in connection with, and the laboratory procedures and techniques of, analytical chemistry, synthetic organic chemistry, and medicinal and pharmaceutical chemistry described herein are those recognized in the field. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods, compounds, compositions described herein.

The terms “kit” and “article of manufacture” are used as synonyms.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” as used herein, include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

As used herein, the term “target protein” refers to a protein or a portion of a protein capable of being bound by, or interacting with a compound described herein, such as a compound with a structure from the group of Compound A. In certain embodiments, a target protein is a STIM protein. In certain embodiments, a target protein is an Orai protein.

As used herein, “STIM protein” includes but is not limited to, mammalian STIM-1, such as human and rodent (e.g., mouse) STIM-1, Drosophila melanogaster D-STIM, C. elegans C-STIM, Anopheles gambiae STIM and mammalian STIM-2, such as human and rodent (e.g., mouse) STIM-2. (see paragraphs through of US 2007/0031814, as well as Table 3 of US 2007/0031814, herein incorporated by reference) As described herein, such proteins have been identified as being involved in, participating in and/or providing for store-operated calcium entry or modulation thereof, cytoplasmic calcium buffering and/or modulation of calcium levels in or movement of calcium into, within or out of intracellular calcium stores (e.g., endoplasmic reticulum).

As used herein, an “Orai protein” includes Orai1 (SEQ ID NO: 1 as described in WO 07/081804), Orai2 (SEQ ID NO: 2 as described in WO 07/081804), or Orai3 (SEQ ID NO: 3 as described in WO 07/081804). Orai1 nucleic acid sequence corresponds to GenBank accession number NM_032790, Orai2 nucleic acid sequence corresponds to GenBank accession number BC069270 and Orai3 nucleic acid sequence corresponds to GenBank accession number NM_152288. As used herein, Orai refers to any one of the Orai genes, e.g., Orai1, Orai2, Orai3 (see Table I of WO 07/081804). As described herein, such proteins have been identified as being involved in, participating in and/or providing for store-operated calcium entry or modulation thereof, cytoplasmic calcium buffering and/or modulation of calcium levels in or movement of calcium into, within or out of intracellular calcium stores (e.g., endoplasmic reticulum).

The term “fragment” or “derivative” when referring to a protein (e.g. STIM, Orai) means proteins or polypeptides which retain essentially the same biological function or activity in at least one assay as the native protein(s). For example, the fragments or derivatives of the referenced protein maintains at least about 50% of the activity of the native proteins, at least 75%, at least about 95% of the activity of the native proteins, as determined e.g. by a calcium influx assay.

As used herein, amelioration of the symptoms of a particular disease, disorder or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

The term “modulate,” as used herein, means to interact with a target protein either directly or indirectly so as to alter the activity of the target protein, including, by way of example only, to inhibit the activity of the target, or to limit or reduce the activity of the target.

As used herein, the term “modulator” refers to a compound that alters an activity of a target. For example, a modulator can cause an increase or decrease in the magnitude of a certain activity of a target compared to the magnitude of the activity in the absence of the modulator. In certain embodiments, a modulator is an inhibitor, which decreases the magnitude of one or more activities of a target. In certain embodiments, an inhibitor completely prevents one or more activities of a target.

As used herein, “modulation” with reference to intracellular calcium refers to any alteration or adjustment in intracellular calcium including but not limited to alteration of calcium concentration in the cytoplasm and/or intracellular calcium storage organelles, e.g., endoplasmic reticulum, and alteration of the kinetics of calcium fluxes into, out of and within cells. In aspect, modulation refers to reduction.

As used herein, the term “target activity” refers to a biological activity capable of being modulated by a modulator. Certain exemplary target activities include, but are not limited to, binding affinity, signal transduction, enzymatic activity, tumor growth, inflammation or inflammation-related processes, and amelioration of one or more symptoms associated with a disease or condition.

The terms “inhibits”, “inhibiting”, or “inhibitor” of SOC channel activity or CRAC channel activity, as used herein, refer to inhibition of store operated calcium channel activity or calcium release activated calcium channel activity.

The term “acceptable” with respect to a formulation, composition or ingredient, as used herein, means having no persistent detrimental effect on the general health of the subject being treated.

The term “pharmaceutically acceptable,” as used herein, refers a material, such as a carrier, diluent, or formulation, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutical combination” as used herein, means a product that results from the mixing or combining of more than one active ingredient and includes both fixed and non-fixed combinations of the active ingredients. The term “fixed combination” means that one active ingredient, e.g. a compound with a structure from the group of Compound A and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutical composition” refers to a mixture of a compound with a structure from the group of Compound A, described herein with other chemical components, such as carriers, stabilizers, diluents, surfactants, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, subcutaneous, intramuscular, pulmonary and topical administration.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition that includes a compound with a structure from the group of Compound A, required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “co-administration” or the like, as used herein, are meant to encompass administration of the selected therapeutic agents to a single patient, and are intended to include treatment regimens in which the agents are administered by the same or different route of administration or at the same or different time.

The term “carrier,” as used herein, refers to relatively nontoxic chemical compounds or agents that facilitate the incorporation of a compound into cells or tissues.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds.

“Bioavailability” refers to the percentage of the weight of the compound disclosed herein (e.g. a compound from the group of Compound A) that is delivered into the general circulation of the animal or human being studied. The total exposure (AUC(0-∞)) of a drug when administered intravenously is usually defined as 100% bioavailable (F %). “Oral bioavailability” refers to the extent to which a compound disclosed herein, is absorbed into the general circulation when the pharmaceutical composition is taken orally as compared to intravenous injection.

“Blood plasma concentration” refers to the concentration of a compound with a structure from the group of Compound A, in the plasma component of blood of a subject. It is understood that the plasma concentration of compounds described herein may vary significantly between subjects, due to variability with respect to metabolism and/or possible interactions with other therapeutic agents. In accordance with one embodiment disclosed herein, the blood plasma concentration of the compounds disclosed herein may vary from subject to subject. Likewise, values such as maximum plasma concentration (Cmax) or time to reach maximum plasma concentration (Tmax), or total area under the plasma concentration time curve (AUC(0-∞)) may vary from subject to subject. Due to this variability, the amount necessary to constitute “a therapeutically effective amount” of a compound may vary from subject to subject.

As used herein, “calcium homeostasis” refers to the maintenance of an overall balance in intracellular calcium levels and movements, including calcium signaling, within a cell.

As used herein, “intracellular calcium” refers to calcium located in a cell without specification of a particular cellular location. In contrast, “cytosolic” or “cytoplasmic” with reference to calcium refers to calcium located in the cell cytoplasm.

As used herein, an effect on intracellular calcium is any alteration of any aspect of intracellular calcium, including but not limited to, an alteration in intracellular calcium levels and location and movement of calcium into, out of or within a cell or intracellular calcium store or organelle. For example, an effect on intracellular calcium can be an alteration of the properties, such as, for example, the kinetics, sensitivities, rate, amplitude, and electrophysiological characteristics, of calcium flux or movement that occurs in a cell or portion thereof. An effect on intracellular calcium can be an alteration in any intracellular calcium-modulating process, including, store-operated calcium entry, cytosolic calcium buffering, and calcium levels in or movement of calcium into, out of or within an intracellular calcium store. Any of these aspects can be assessed in a variety of ways including, but not limited to, evaluation of calcium or other ion (particularly cation) levels, movement of calcium or other ion (particularly cation), fluctuations in calcium or other ion (particularly cation) levels, kinetics of calcium or other ion (particularly cation) fluxes and/or transport of calcium or other ion (particularly cation) through a membrane. An alteration can be any such change that is statistically significant. Thus, for example if intracellular calcium in a test cell and a control cell is said to differ, such difference can be a statistically significant difference.

As used herein, “involved in” with respect to the relationship between a protein and an aspect of intracellular calcium or intracellular calcium regulation means that when expression or activity of the protein in a cell is reduced, altered or eliminated, there is a concomitant or associated reduction, alteration or elimination of one or more aspects of intracellular calcium or intracellular calcium regulation. Such an alteration or reduction in expression or activity can occur by virtue of an alteration of expression of a gene encoding the protein or by altering the levels of the protein. A protein involved in an aspect of intracellular calcium, such as, for example, store-operated calcium entry, thus, can be one that provides for or participates in an aspect of intracellular calcium or intracellular calcium regulation. For example, a protein that provides for store-operated calcium entry can be a STIM protein and/or an Orai protein.

As used herein, a protein that is a component of a calcium channel is a protein that participates in multi-protein complex that forms the channel.

As used herein, “basal” or “resting” with reference to cytosolic calcium levels refers to the concentration of calcium in the cytoplasm of a cell, such as, for example, an unstimulated cell, that has not been subjected to a condition that results in movement of calcium into or out of the cell or within the cell. The basal or resting cytosolic calcium level can be the concentration of free calcium (i.e., calcium that is not bound to a cellular calcium-binding substance) in the cytoplasm of a cell, such as, for example, an unstimulated cell, that has not been subjected to a condition that results in movement of calcium into or out of the cell.

As used herein, “movement” with respect to ions, including cations, e.g., calcium, refers to movement or relocation, such as for example flux, of ions into, out of, or within a cell. Thus, movement of ions can be, for example, movement of ions from the extracellular medium into a cell, from within a cell to the extracellular medium, from within an intracellular organelle or storage site to the cytosol, from the cytosol into an intracellular organelle or storage site, from one intracellular organelle or storage site to another intracellular organelle or storage site, from the extracellular medium into an intracellular organelle or storage site, from an intracellular organelle or storage site to the extracellular medium and from one location to another within the cell cytoplasm.

As used herein, “cation entry” or “calcium entry” into a cell refers to entry of cations, such as calcium, into an intracellular location, such as the cytoplasm of a cell or into the lumen of an intracellular organelle or storage site. Thus, cation entry can be, for example, the movement of cations into the cell cytoplasm from the extracellular medium or from an intracellular organelle or storage site, or the movement of cations into an intracellular organelle or storage site from the cytoplasm or extracellular medium. Movement of calcium into the cytoplasm from an intracellular organelle or storage site is also referred to as “calcium release” from the organelle or storage site.

As used herein, “protein that modulates intracellular calcium” refers to any cellular protein that is involved in regulating, controlling and/or altering intracellular calcium. For example, such a protein can be involved in altering or adjusting intracellular calcium in a number of ways, including, but not limited to, through the maintenance of resting or basal cytoplasmic calcium levels, or through involvement in a cellular response to a signal that is transmitted in a cell through a mechanism that includes a deviation in intracellular calcium from resting or basal states. In the context of a “protein that modulates intracellular calcium,” a “cellular” protein is one that is associated with a cell, such as, for example, a cytoplasmic protein, a plasma membrane-associated protein or an intracellular membrane protein. Proteins that modulate intracellular calcium include, but are not limited to, ion transport proteins, calcium-binding proteins and regulatory proteins that regulate ion transport proteins.

As used herein, “cell response” refers to any cellular response that results from ion movement into or out of a cell or within a cell. The cell response may be associated with any cellular activity that is dependent, at least in part, on ions such as, for example, calcium. Such activities may include, for example, cellular activation, gene expression, endocytosis, exocytosis, cellular trafficking and apoptotic cell death.

As used herein, “immune cells” include cells of the immune system and cells that perform a function or activity in an immune response, such as, but not limited to, T-cells, B-cells, lymphocytes, macrophages, dendritic cells, neutrophils, eosinophils, basophils, mast cells, plasma cells, white blood cells, antigen presenting cells and natural killer cells.

As used herein, “cytokine” refers to small soluble proteins secreted by cells that can alter the behavior or properties of the secreting cell or another cell. Cytokines bind to cytokine receptors and trigger a behavior or property within the cell, for example, cell proliferation, death or differentiation. Exemplary cytokines include, but are not limited to, interleukins (e.g., IL-2, IL-3, IL-4, IL-5, IL-6, IL-7, IL-8, IL-9, IL-10, IL-11, IL-12, IL-13, IL-15, IL-16, IL-17, IL-18, IL-1a, IL-1ß, and IL-1 RA), granulocyte colony stimulating factor (G-CSF), granulocyte-macrophage colony stimulating factor (GM-CSF), oncostatin M, erythropoietin, leukemia inhibitory factor (LIF), interferons, B7.1 (also known as CD80), B7.2 (also known as B70, CD86), TNF family members (TNF-α, TNF-β, LT-β, CD40 ligand, Fas ligand, CD27 ligand, CD30 ligand, 4-1BBL, Trail), and MIF.

“Store operated calcium entry” or “SOCE” refers to the mechanism by which release of calcium ions from intracellular stores is coordinated with ion influx across the plasma membrane.

“Selective inhibitor of SOC channel activity” means that the inhibitor is selective for SOC channels and does not substantially affect the activity of other types of ion channels.

“Selective inhibitor of CRAC channel activity” means that the inhibitor is selective for CRAC channels and does not substantially affect the activity of other types of ion channels and/or other SOC channels.

As used herein, the term “calcium” may be used to refer to the element or to the divalent cation Ca²⁺.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1: Efficacy and Recovery of Auxora Compared to Placebo

Tables 1-4 below provide the data for patients treated with placebo or Auxora. Table 1 summarizes the efficacy data for mortality between patients given placebo compared to patients treated with Auxora through 60 days.

TABLE 1
Efficacy Set Mortality Through Day 60

| Mortality | Number of | | | |
|---|---|---|---|---|
| Day 60 | Patients | Placebo | Auxora | Chi-squared test |
| All Treated | 280 | 27/140 | 17/140 | Difference: −7.04 |
| Set | | (19.3%) | (12.1%) | (95% CI): (−15.36, 1.28) |
| | | | | P-value: 0.1001 |

Efficacy Set by Ventilation Mode Subgroups

| | | | | |
|---|---|---|---|---|
| HFNC | 162 | 21/82 | 13/80 | Difference: −9.36 |
| | | (25.6%) | (16.0%) | (95% CI): (−21.79, 3.07) |
| | | | | P−value: 0.1436 |
| LFO₂ | 98 | 6/49 | 5/49 | Difference: −2.04 |
| | | (12.2%) | (10.2%) | (95% CI): (−14.53, 10.45) |
| | | | | P−value: 0.7490 |

Efficacy Set by Imputed PF Subgroups

| | | | | |
|---|---|---|---|---|
| PF ≤100 | 116 | 17/58 | 12/58 | Difference: −8.62 |
| | | (29.3%) | (20.3%) | (95% CI): (−24.30, 7.06) |
| | | | | P−value: 0.2837 |
| PF 101-200 | 144 | 10/73 | 6/71 | Difference: −5.25 |
| | | (13.7%) | (8.5%) | (95% CI): (−15.45, 4.95) |
| | | | | P-value: 0.3164 |

Table 2 summarizes the demographics for the study comparing patients treated with placebo and patients treated with Auxora.

TABLE 2
Efficacy Set Demographics

| | PLACEBO | AUXORA |
|---|---|---|
| Number of Patients | 131 | 130 |
| Male % | 70.2% | 64.6% |
| White % | 74.8% | 65.4% |
| Median Age | 61 | 60 |
| Median BMI | 31.0 | 31.1 |
| Median Time from Symptom | 12.0 days | 11.0 days |
| Onset | | |
| % HFNC | 62.6% | 62.3% |
| Median Screening PF value | 104.0 | 106.7 |
| PF ≤100 | 44.3% | 45.4% |
| Median CRP | 74.0 | 69.8 |

Table 3 summarizes the time of recovery for patients treated with placebo compared to Auxora at the primary endpoint. As shown in the table below, patients treated with Auxora have three days less recovery compared to patients who were treated with the placebo.

TABLE 3
Time to Recovery

| | | | | Median Days | | | |
|---|---|---|---|---|---|---|---|
| | Number of | | | Placebo vs. | | Recovery | |
| Recovery | Patients | Placebo | Auxora | Auxora | P-Value | Rate Ratio | 95% CI |
| Efficacy Set | 261 | 74% | 79.2% | 10.0 vs. 7.0 | 0.098 | 1.25 | 0.95, 1.65 |
| All Treated | 281 | 75.7% | 80.9% | 8.0 vs. 7.0 | 0.042 | 1.30 | 1.00, 1.69 |
| Patient Set | | | | | | | |

Table 4 summarizes the safety data of placebo compared to Auxora. Patients that exhibited serious adverse events (SAEs) are compared in Table 4 below between patients treated with placebo and patients treated with Auxora.

TABLE 4
Safety Analysis

| | Placebo | Auxora |
|---|---|---|
| Number of Patients Safety Set | 140 | 141 |
| Number of Patients with SAEs | 49 (35.0%) | 34 (24.1%) |
| Number of SAEs | 101 | 75 |

Number of Patients with SAEs by Preferred Term (≥3%)

| | | |
|---|---|---|
| Respiratory Failure | 26 (18.6%) | 22 (15.6%) |
| ARDS | 11 (7.9%) | 7 (5.0%) |
| Pneumonia | 7 (5.0%) | 6 (4.3%) |
| Cardiac Arrest | 6 (4.3%) | 6 (4.3%) |
| Septic Shock | 8 (5.7%) | 2 (1.4%) |

Study Drug Discontinuation Due to AE/Toxicity

| | | |
|---|---|---|
| Number of Patients | 5 (3.6%) | 3 (2.1%) |

Recent results from human clinical studies show that intravenous Auxora is safe in critically ill patients and may reduce the severity and duration of respiratory failure. -2-yl)-2-fluoro-6-methylbenzamide-injection emulsion (IE) compared to controls. Both the preclinical and clinical data support the development of Auxora for use in patients with ALI/ARDS secondary to COVID-19 infection.

### Example 2: Phase 2 Clinical Trials

A phase 2 clinical trial is performed to evaluate the safety, tolerability, pharmacokinetics and pharmacodynamics of the pharmaceutical compositions disclosed herein for extended treatment on subjects having ALI and/ARDS or at risk for developing ALI and/or ARDS, such as contracted COVID-19 or other respiratory viruses/bacteria that are likely to lead to ALI/ARDS.

Number of Patients and Sites: Up to 240 patients with confirmed COVID-19 pneumonia with a baseline imputed PaO2/FiO2 ≤200 receiving oxygen therapy via a high flow nasal cannula (HFNC) or non-invasive ventilation (NIV) at up to approximately 40 enrolling sites.

Auxora and Placebo Dose and Route of Administration: All patients will receive 2.0 mg/kg (1.25 mL/kg) of Auxora at 0 hour and 1.6 mg/kg (1 mL/kg) at both 24 hours and 48 hours from the Start of the First Infusion of Auxora (SFIA) at 0 hour. Patients with severe hypoxemic respiratory failure, those on HFNC with a recorded worst imputed PaO₂/FiO₂ $100 between the end of the infusion of Auxora at 24 hours and the start of the infusion of Auxora at 48 hours or those on NIV or invasive mechanical ventilation (IMV) at the start of the infusion of Auxora at 48 hours, will be randomized 1:1 to receive 1.6 mg/kg (1 mL/kg) of Auxora or 1 mL/kg of Placebo at 72, 96, and 120 hours from the SFIA. All doses of Auxora and Placebo will be administered intravenously as a continuous infusion over 4 hours via a bag and tubing compatible with lipid emulsions and using a 1.2-micron filter.

Hypothesis: The pathophysiological course of COVID-19 pneumonia proceeds in two distinct phases; an initial viral replication followed by an excessive, dysregulated host immune response leading to alveolitis and hypoxemic respiratory failure. Current evidence suggests that alveolitis results from the positive feedback loop between monocyte-derived alveolar macrophages and T cells. Tissue resident alveolar macrophages (TRAMs) respond to SARSCoV-2 infection in the lung by producing T-cell chemoattractants. Arriving T cells produce interferon-gamma (IFN γ), leading to further alveolar macrophage activation and recruitment of monocyte-derived alveolar macrophages. The feedback loop leads to a rapid increase in proinflammatory cytokines, diffuse alveolar injury, and severe endothelialitis leading to acute respiratory disease syndrome (ARDS), and multi-organ dysfunction and failure.

Calcium Release-Activated Calcium (CRAC) channels play important pathogenic roles in the several cell types and pathways linked to COVID-19 pneumonia. Activation of CRAC channels stimulates the production and release of pro-inflammatory cytokines from immune cells, including those elevated by SARS-CoV-2 infection (e.g., IFN-γ, IL-6, IL-17 and TNF α). Pathophysiological activation of CRAC channels has also been associated with pulmonary endothelial cell dysfunction and plasma extravasation in animal models of acute lung injury. Finally, although the role of CRAC channels in monocyte functioning is still emerging, release of reactive oxygen species from monocytes has been shown to be controlled by Orai1 CRAC channels.

The inhibition of CRAC channels may provide the kind of broad-based approach likely to be effective in treating patients with critical COVID-19 pneumonia. Auxora, a calcium release-activated calcium (CRAC) channel inhibitor potently blocks the production and release of pro-inflammatory cytokines from immune cells, including those elevated by SARS-CoV-2 infection. Auxora reduced inflammation and plasma extravasation in an animal model of acute lung injury, suggesting preservation of endothelial integrity. In addition, since the Ca²⁺ entering through CRAC channels in T cells primarily activates the calcineurin/NFAT signal transduction pathway, Auxora may act cooperatively with anti-inflammatory drugs such as dexamethasone that work through the NF-κ B signal transduction pathway. Auxora is given intravenously, is distributed into the lung within 2 to 4 hours of the start of infusion, has a rapid onset of activity with IL-2 production being decreased by >50% at the end of the infusion, and does not appear to have long term immune-modulatory effects, with recovery of IL-2 production 24 hours after the end of the infusion.

Auxora has been tested in a randomized, double blind, placebo-controlled trial of patients with severe COVID-19 pneumonia being treated with corticosteroids (CARDEA; NCT04345614). In patients with baseline imputed PaO2/FiO2≤200 (n=261), the median time to recovery was 7 versus 10 days (P=0.0979) for patients who received Auxora versus placebo, respectively. The all-cause mortality rate at Day 60 in these patients was 13.8% with Auxora versus 20.6% with placebo (p=0.1449); Day 30 all-cause mortality rate was 7.7% and 17.6%, respectively (p=0.0165). The all-cause mortality rate at Day 60 in the subgroup of patients receiving oxygen therapy with HFNC at baseline (n=162) was 16.0% with Auxora versus 25.6% with placebo (p=0.1436). A similar reduction at Day 60 was noted in the subgroup of patients with a baseline imputed PaO2/FiO2≤100 (n=117): 20.3% with Auxora and 29.3% with Placebo. The proportion of patients with serious adverse events (SAEs) was less in patients treated with Auxora (24.1%) compared to placebo (35.0%) and fewer patients discontinued study drug because of an adverse event (AE) or toxicity with Auxora (n=3) versus placebo (5).

In CARDEA, 81 patients on HFNC at baseline were treated with Auxora. 8 of the 81 patients were on IMV at the start of the infusion at 48 hours. 4 of the 8 patients subsequently died. 39 patients had a worst imputed PaO2/FiO2 >100 between the end of the infusion of Auxora at 24 hours and the start of the infusion of Auxora at 48 hours. Only 2 of the 39 patients subsequently needed IMV or died. 34 patients had a worst imputed PaO2/FiO2 ≤100 between the end of the infusion of Auxora at 24 hours and the start of the infusion of Auxora at 48 hours. 13 of the 34 patients subsequently needed IMV and or died.

The data from CARDEA suggest the potential benefit and safety of Auxora in patients with severe and critical COVID-19 pneumonia. It also reveals a subgroup of patients who have a high risk of disease progression and death and may benefit from additional days of dosing of Auxora. This study, therefore, aims to further investigate the safety and clinical efficacy of Auxora in patients with critical COVID-19 pneumonia as standard of care continues to evolve to include other immunomodulators beyond corticosteroids, as well as the clinical efficacy of six versus three doses of Auxora in patients at high risk of disease progression and death.

Primary objectives: To assess the safety and tolerability of Auxora when administered in patients receiving corticosteroids and tocilizumab as standard of care. To assess the clinical efficacy of 6 doses versus 3 doses of Auxora in randomized patients with severe hypoxemic respiratory failure, those on HFNC with a recorded worst imputed PaO2/FiO2≤100 between the end of the infusion of Auxora at 24 hours and the start of the infusion of Auxora at 48 hours or those on NIV or IMV at the start of the infusion of Auxora at 48 hours.

Secondary objectives: To assess the safety and tolerability of Auxora in patients with critical COVID-19 pneumonia receiving oxygen therapy via a HFNC or NIV at Baseline. To assess the pharmacokinetic profile of Auxora in patients with critical COVID-19 pneumonia.

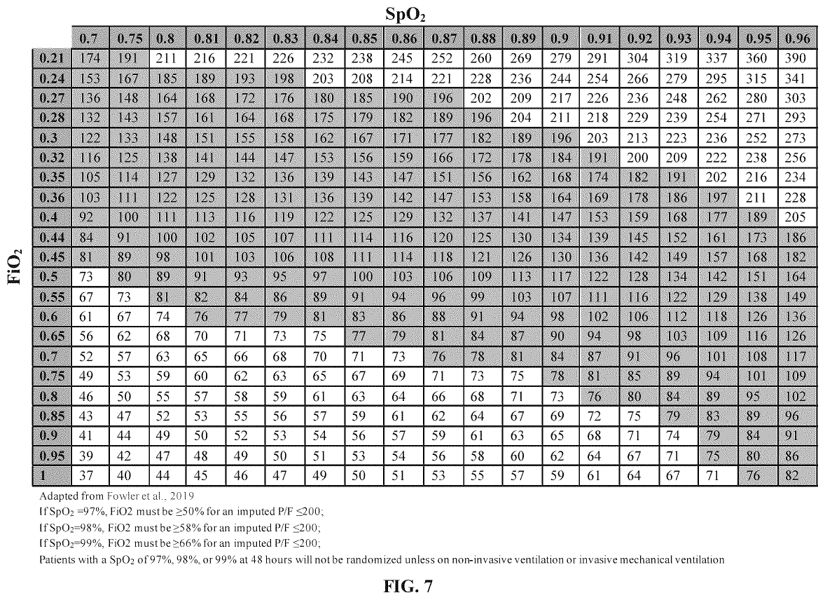

Inclusion Criteria: all of the following must be met for a patient to be enrolled into the study:
1. Has laboratory-confirmed SARS-CoV-2 infection as determined by polymerase chain reaction (PCR) or other commercial or public health assay in any specimen, as documented by either of the following:
   PCR positive in sample collected <72 hours prior to consent;
   PCR positive in sample collected ≥72 hours prior to consent, with inability to obtain a repeat sample (e.g. due to lack of testing supplies, or limited testing capacity, or results taking >24 hours, etc.) or progressive disease suggestive of ongoing SARS-CoV-2 infection;
2. At least 1 of the following symptoms:
   Fever, cough, sore throat, malaise, headache, muscle pain, dyspnea at rest or with exertion, confusion, or respiratory distress;
3. A PaO₂/FiO₂$200 recorded in the 24 hours before consent. The PaO₂/FiO₂ can be imputed from pulse oximetry (FIG. 7);
4. Oxygen therapy being administered via HFNC or NIV
5. The presence of a respiratory infiltrate or abnormality consistent with pneumonia that is documented by either a CXR or CT scan of the lungs;
6. The patient is ≥18 years of age;
7. A female patient of childbearing potential must not attempt to become pregnant for 180 days, and if sexually active with a male partner, is willing to practice acceptable methods of birth control for 180 days after the last dose of study drug;
8. A male patient who is sexually active with a female partner of childbearing potential is willing to practice acceptable methods of birth control for 180 days after the last dose of study drug. A male patient must not donate sperm for 180 days;
9. The patient is willing and able to, or has a legal authorized representative (LAR) who is willing and able to, provide informed consent to participate, and to cooperate with all aspects of the protocol.

Exclusion criteria: patients with any of the following conditions or characteristics at Screening must be excluded from enrolling:
1. Do Not Intubate order;
2. PaO₂/FiO₂≤75 recorded at the time of consent. The PaO₂/FiO₂ can be imputed from pulse oximetry (FIG. 7);
3. Receiving IMV via endotracheal intubation or tracheostomy;
4. Receiving ECMO;
5. Shock defined by the use of vasopressors;
6. Known history of:
   a. Organ or hematologic transplant;
   b. HIV;
   c. Active hepatitis B, or hepatitis C infection;
7. Current treatment with:
   a. Chemotherapy;
   b. Immunosuppressive medications or immunotherapy (see list below of prohibited immunosuppressive medications and immunotherapy) at the time of consent;
   c. Hemodialysis or Peritoneal Dialysis;
8. Known to be pregnant or is nursing;
9. Currently participating in another study of an investigational drug or therapeutic medical device at the time of consent;
10. Known allergy to eggs or any of the excipients in study drug.

Study Design: All patients who meet all of the inclusion criteria and none of the exclusion criteria will receive three doses of Auxora. The dose of Auxora will be 2.0 mg/kg (1.25 mL/kg) administered at 0 hour, and then 1.6 mg/kg (1 mL/kg) at 24 hours and 1.6 mg/kg (1 mL/kg) at 48 hours from the SFIA.

Patients with severe hypoxemic respiratory failure, those on HFNC with a recorded worst imputed PaO2/FiO2 ≤100 between the end of the infusion of Auxora at 24 hours and the start of the infusion of Auxora at 48 hours or are on NIV or IMV at the start of the infusion of Auxora at 48 hours, will be randomized 1:1 to receive 1.6 mg/kg (1 mL/kg) of Auxora or 1 mL/kg of Placebo at 72, 96 and 120 hours from the SFIA. Patients on IMV will be stratified between the Auxora and Placebo groups.

Patients without severe hypoxemic respiratory failure, those with a recorded worst imputed PaO2/FiO2 >100 between the end of the infusion of Auxora at 24 hours and the start of the infusion of Auxora at 48 hours and are not on NIV or IMV, will not be randomized for further infusions but will remain in the study and complete all assessments through Day 60.

The dosing of Auxora and Placebo will be based on actual body weight obtained at the time of hospitalization or screening for the study. As described in the pharmacy manual, there will be an upper limit of the absolute dose (volume) of Auxora and volume of Placebo that will be administered for patients weighing more than 125 kg.

A study physician or appropriately trained delegate will perform assessments at Screening, immediately prior to the SFIA, and immediately prior to each subsequent infusion at 24 and 48 hours from the SFIA in all patients.

In patients randomized to receive additional doses of study drug, assessments will be performed prior to each subsequent infusion at 72, 96 and 120 hours from the SFIA. Starting at 144 hours from the SFIA, the patients will then be assessed every 24 hours (±4 hours) until 336 hours from the SFIA, then q48 hours until discharge or Day 60. All patients discharged before Day 25 will be followed-up at Day 30 (±5 days) for a safety and mortality assessment; all patients who are discharged on or after Day 25 but before Day 30 will use the discharge assessment as the Day 30 assessment. All patients will be followed up at Day 60 (±5 days) for a safety and mortality assessment; all patients who are discharged on or after Day 55 but before Day 60 will use the discharge assessment as the Day 60 assessment.

For patients not randomized to receive additional doses of study drug, patients will be assessed every 24 hours (±4 hours) starting at 72 hours from the SFIA and until 336 hours from the SFIA, then q48 hours until discharge or Day 60. All patients discharged before Day 25 will be followed-up at Day 30 (±5 days) for a safety and mortality assessment; all patients who are discharged on or after Day 25 but before Day 30 will use the discharge assessment as the Day 30 assessment. All patients will be followed up at Day 60 (±5 days) for a safety and mortality assessment; all patients who are discharged on or after Day 55 but before Day 60 will use the discharge assessment as the Day 60 assessment.

After approximately the first 50 patients are enrolled in the study, and then again after approximately 100 and 200 patients are enrolled in the study, an Independent Data Monitoring Committee (IDMC) will evaluate the safety data from the study. Information regarding the IDMC review and timing of IDMC meetings will be further detailed in the IDMC Charter for the study.

All patients enrolled in the study should receive care consistent with local standard of care. Patients with worsening respiratory failure should receive conservative intravenous fluid strategies such as FACTT LITE. All patients should receive pharmacological prophylaxis to prevent the development of venous thromboembolic disease. The type and dose of prophylaxis should be determined by local standard of care.

Patients enrolled in the study should receive dexamethasone, or equivalent dose of another corticosteroid, as standard of care. If patients are not receiving dexamethasone at the time of enrollment into CARDEA-Plus, dexamethasone or the equivalent dose of another steroid should be started. If the patient is already receiving dexamethasone at the time of enrollment, dexamethasone should be continued on its established dosing schedule. The COVID-19 Treatment Guidelines Panel of the National Institutes of Health recommends using dexamethasone (at a dose of 6 mg per day, given orally or intravenously, for up to 10 days) in patients with COVID-19 who require supplemental oxygen. Of note, the dose equivalencies for dexamethasone 6 mg daily are prednisone 40 mg daily, methylprednisolone 32 mg daily, and hydrocortisone 160 mg daily.

If patients are not receiving remdesivir at the time of enrollment into CARDEA-Plus, starting remdesivir during the hospitalization may be considered. If the patient is already receiving remdesivir at the time of enrollment, remdesivir should be continued on its established dosing schedule. The suggested dose of remdesivir for adults weighing ≥40 kg and not requiring invasive mechanical ventilation and/or ECMO is a single dose of 200 mg infused intravenously over 30 to 120 minutes on Day 1 followed by once-daily maintenance doses of 100 mg infused intravenously over 30 to 120 minutes for 4 days (days 2 through 5). If a patient does not demonstrate clinical improvement, treatment may be extended for up to 5 additional days

(i.e., up to a total of 10 days). Auxora and remdesivir should not infuse at the same time but should be given sequentially but may be given in any order.

For patients who within 3 days of hospitalization have rapidly increasing oxygen needs and evidence for systemic inflammation, the administration of tocilizumab, 8 mg/kg actual body weight (up to 800 mg) administered as a single IV dose, may be considered. Although approximately a third of patients in the REMAP-CAP and RECOVERY trials received a second dose of tocilizumab at the discretion of the treating physicians, data on outcomes based on receipt of one or two doses is not available. Therefore, there is insufficient evidence to determine which patients, if any, would benefit from an additional dose of tocilizumab.

Other immunosuppressive medications or immunotherapies are prohibited in patients enrolled into the study. (See list of prohibited medications below). The use of dextromethorphan is discouraged in patients enrolled into the study.

Prohibited list of immunosuppressive medications and immunotherapy:

chemotherapy, cyclosporine, tacrolimus, sirolimus, everolimus, azathioprine, cyclosphosphamide, methotrexate, mycophenolate, leflunomide, abatacept, adalimumab, alemtuzumab, anakinra, basilizimab, belimumab, bevacizumab, brodalumab, canakinumab, certolizumab, cetuximab, clazakizumab, daclizumab, eculizumab, etanercept, golimumab, infliximab, interferon, ixekizumab, muromonab, natalizumab, omalizumab, rituximab, sarilumab, secukinumab, trastuzumab, ustekinumab, vedolizumab, baricitinib, tofacitinib, or other janus kinase inhibitors. Further use of dextromethorphan is discouraged.

Efficacy endpoints: efficacy endpoints will include the following:

### Primary Efficacy Endpoint

Proportion of randomized patients receiving 6 versus 3 doses of Auxora requiring IMV after 72 hours from the SFIA or dying through Day 60 Secondary Efficacy Endpoints.
All-Cause Mortality through Day 60 in randomized patients receiving 6 versus 3 doses of Auxora
All-Cause Mortality through Day 30 in randomized patients receiving 6 versus 3 doses of Auxora
Proportion of randomized patients receiving 6 versus 3 doses of Auxora
requiring IMV after 72 hours from the SFIA through Day 60
Number of Days in the Hospital
Number of Days in the ICU

### Exploratory Endpoints

Reduction in Angiopoietin 2 levels after treatment with Auxora
Increase in Angiopoietin 1 levels after treatment with Auxora

Safety Endpoints: safety endpoints will include the following:

### Safety Endpoints Will Include the Following:

The incidence of TEAEs and SAEs
The intensity and relationship of TEAEs and SAEs
Clinically significant changes in vital signs and safety laboratory results

Sample size calculation: In the Randomized Stage, with a 1:1 treatment allocation ratio (Auxora:placebo), a two group chi-squared test with a 5% two-sided significance level will have 70% power to detect the difference in requiring IMV after 72 hours from the SFIA or dying through Day 60 between a Placebo proportion of 42% and an Auxora proportion of 20% (odds ratio of 0.35) when the sample size in each group is 54. Sample size for the Randomized Stage was estimated using nQuery v8.4 by statistical solutions ltd.

Based on CARDEA, the percent of enrolled patients on HFNC with a recorded worst imputed PaO2/FiO2≤100 between the end of the infusion of Auxora at 24 hours and the start of the infusion of Auxora at 48 hours or those on NIV or invasive mechanical ventilation (IMV) at the start of the infusion of Auxora at 48 hours is estimated to be 50%. In order to fulfill the need for 54 subjects randomized to the Auxora and Placebo groups, it is anticipated that 216 subjects total will be need to be enrolled. With 10% of early drop rate, approximately 240 subjects will be enrolled in the study.

### CARDEA

CARDEA was a phase 2, randomized, double blind, placebo-controlled trial evaluating the addition of Auxora, a CRAC channel inhibitor, to corticosteroids and standard of care in adults with severe COVID-19 pneumonia. The primary endpoint was time to recovery through Day 60, with recovery being defined as being in one of three categories: hospitalized not requiring supplemental oxygen or ongoing medical care, discharged requiring supplemental oxygen, and discharged not requiring supplemental oxygen. Key secondary endpoints were all cause mortality at Day 60 and Day 30. Due to declining rates of US COVID-19 hospitalizations and encroachment of prohibited medications as standard of care, the study was stopped early.

In total, 143 patients were randomized to Auxora and 141 to placebo. 100% of patients in both the Auxora and placebo groups received corticosteroids. The median time to recovery in patients receiving ≥1 dose of study drug (n=281) was 7 versus 8 days (P=0.0420) for those receiving Auxora versus placebo, respectively. Time to recovery in patients with baseline imputed PaO2/FiO2≤200 (n=261) was 7 days for those receiving Auxora (n=130) versus 10 days for those receiving placebo (n=131) (P=0.0979). The all-cause mortality rate at Day 60 in patients with a baseline imputed PaO2/FiO2 ≤200 was 13.8% for those receiving Auxora versus 20.6% for those receiving placebo (P=0.1449); Day 30 all-cause mortality was 7.7% and 17.6%, respectively (P=0.0165).

Similar trends in faster recovery and lower mortality were noted in a pre-specified subgroup of patients on HFNC at baseline (n=163). 81 patients were randomized to Auxora and 82 patients were randomized to placebo. The median time to recovery for those receiving Auxora was 9 days versus 17 days for those receiving placebo (P=0.1079). Day 60 all-cause mortality rate for patients on HFNC at baseline receiving Auxora was 16% versus 25.6% for those receiving placebo (P=0.1436).

Auxora was found to be safe and well tolerated in CARDEA, with fewer patients experiencing serious adverse events and adverse events necessitating discontinuation of dosing. In total, 34 patients (24.1%) receiving Auxora and 49 (35.0%) receiving placebo experienced SAEs. The most common were respiratory failure, ARDS, and pneumonia. Discontinuation due to TEAEs occurred in 3 patients receiving Auxora and 5 patients receiving placebo (Table 5).

TABLE 5
Serious Adverse Events and Study Drug Discontinuation

| | Placebo | Auxora |
|---|---|---|
| Number of Patients | 140 | 141 |

Study Drug Discontinuation

| | | |
|---|---|---|
| AE/Toxicity | 5 | 3 |

SAEs by Preferred Term ≥4%

| | | |
|---|---|---|
| Number of SAEs | 101 | 75 |
| Number of Patients | 49 (35.0%) | 34 (24.1%) |
| Respiratory Failure | 26 (18.6%) | 22 (15.6%) |
| ARDS | 11 (7.9%) | 7 (5.0%) |
| Pneumonia | 7 (5.0%) | 6 (4.3%) |
| Cardiac Arrest | 6 (4.3%) | 6 (4.3%) |
| Septic Shock | 8 (5.7%) | 2 (1.4%) |

The most common TEAEs were respiratory failure, increasing triglycerides, hyperglycemia, and acute kidney injury. All cases of increased triglycerides in both the Auxora and placebo groups were classified as mild. There were no cases of hyperglycemia reported as being severe (Table 6).

TABLE 6
Treatment Emergent Adverse Events

| | |
|---|---|
| Placebo | Auxora |

TEAEs by Preferred Term ≥4%

| | | |
|---|---|---|
| Respiratory Failure | 26 (18.6%) | 22 (15.6%) |
| Blood Triglycerides Increased | 5 (3.6%) | 16 (11.3%) |
| Hypertriglyceridemia | 4 (2.9%) | 2 (1.4%) |
| Hyperglycemia | 11 (7.9%) | 11 (7.8%) |
| Acute Kidney Injury | 16 (11.4%) | 10 (7.1%) |
| Increased Transaminases | 5 (3.6%) | 8 (5.7%) |
| Liver Function Test Increased | 1 (0.7%) | 5 (3.5%) |
| ARDS | 11 (7.9%) | 7 (5.0%) |
| DVT | 7 (5.0%) | 7 (5.0%) |
| Pneumonia | 7 (5.0%) | 7 (5.0%) |
| Pneumothorax | 6 (4.3%) | 7 (5.0%) |
| Pneumomediastinum | 2 (1.4%) | 6 (4.3%) |
| Hypoxia | 7 (5.0%) | 6 (4.3%) |
| Cardiac Arrest | 6 (4.3%) | 6 (4.3%) |
| Hyperkalemia | 6 (4.3%) | 4 (2.8%) |
| Anemia | 9 (6.4%) | 3 (2.1%) |
| Septic Shock | 13 (9.3%) | 2 (1.4%) |

Mechanistically, CRAC-channel inhibitors, such as Auxora, may have therapeutic efficacy in both hastening recovery and reducing mortality in severe COVID-19 pneumonia, and as such, warrant continued clinical development. Results from CARDEA demonstrated that Auxora was safe and well tolerated with strong signals in both time to recovery and all-cause mortality. The benefit may have been in part due to synergy with corticosteroids administered as standard of care. In T cells, CRAC channels are known to be a proximal component of the calcium dependent pathway leading to activation of calcineurin and NFAT, the transcription factor that controls production of IL-2 and other cytokines. CRAC channel inhibitors, therefore, act partly by decreasing the activation of NFAT in stimulated T cells. Corticosteroids such as dexamethasone, on the other hand, are thought to mediate their immunomodulatory effects through a different transcription factor, NF-κB (Grundy et al., Clin Sci, 2014), therefore their effects on inflammatory cells may be complimentary with those of CRAC channel inhibitors.

Tocilizumab and baricitinib are now recommended for recently hospitalized patients with COVID-19 pneumonia with rapidly increasing oxygen needs and evidence for systemic inflammation. It is likely that CRAC channel inhibition may also have synergy with IL-6 receptor blockers in improving outcomes in critically ill patients with COVID-19 pneumonia by lowering the production of multiple proinflammatory cytokines, such as IL-17 and IFN Y, all of which have been implicated in the development of SARS-CoV-2 induced alveolitis (Parrot et al., Sci Immunol, 2020; Grant et al, Nature, 2021). The potential synergy with baricitinib is unclear at this time.

The immunomodulatory effects of Auxora are not expected to be additive with an IL-6 blocker such as tocilizumab. This potential for Auxora to add to the immunomodulatory effects of baricitinib is uncertain at this time and is the reason why the use of baricitinib is excluded from the study. Based on PK/PD data in humans, the recommended dose of tocilizumab of 8 mg/kg is expected to inhibit IL-6 binding to its receptor by more than 95%, essentially negating IL-6 signaling, as long as serum tocilizumab levels remain at or above 1 μg/mL-even in the face of tocilizumab-induced increases in serum IL-6 levels. It stands to reason, therefore, that any inhibition of IL-6 production and release produced by Auxora (CM4620) will not augment the inhibition of IL-6 signaling already produced by tocilizumab or any other IL-6 blocker at comparable doses. Thus, the immunomodulatory effects of Auxora mediated by a reduction in IL-6 signaling is not expected to be additive or synergistic with an IL-6 blocker such as tocilizumab.

The pharmacodynamic study in patients with acute pancreatitis showed that the effects of Auxora on immune cells are moderate in nature and are rapidly reversed with cessation of therapy. There were no increased serious adverse events related to secondary infections with Auxora treatment noted in CARDEA. It is, however, justified conducting this study with the objective of documenting the safety of Auxora when used in combination with corticosteroids and tocilizumab. The risk benefit ratio favors studying this combination therapy in patients with the highest risk of mortality, those with critical COVID-19 pneumonia requiring oxygen therapy with non-invasive ventilation or HFNC.

In CARDEA, 81 patients on HFNC at baseline were treated with Auxora. 8 of the 81 patients were on IMV at the start of the infusion at 48 hours. 4 of the 8 patients subsequently died. 39 patients had a worst imputed PaO2/FiO2 >100 between the end of the infusion of Auxora at 24 hours and the start of the infusion of Auxora at 48 hours. Only 2 of the 39 patients subsequently needed IMV or died. 34 patients had a worst imputed PaO2/FiO2≤100 between the end of the infusion of Auxora at 24 hours and the start of the infusion of Auxora at 48 hours. 13 of the 34 patients subsequently needed IMV and or died. High risk patients on HFNC with a PaO2/FiO2≤100 before the third infusion of Auxora, or on NIV or IMV before the third infusion of Auxora, may potentially benefit from additional days of dosing of Auxora.

PK modeling suggests that administration of Auxora for 6 days would allow continued treatment of these high-risk patients with an acceptable safety margin. Results of PK sample analysis from CARDEA were integrated into a Population-PK model that had been generated from the SAD, MAD, acute pancreatitis studies, and the initial open-label study in COVID-19. Modeling suggests that with six days of dosing, the initial dose being 2 mg/kg and subsequent dosing of 1.6 mg/kg/day, the mean AUC24 h at Day 6 will still provide a greater than 3 fold safety margin compared to the NOAEL level determined from preclinical monkey toxicology studies and the maximum AUC24 h level would remain below the NOAEL level.

The two primary objectives of the study, therefore, are to assess the safety and tolerability of Auxora when administered in patients receiving corticosteroids and tocilizumab as standard of care and to assess the clinical efficacy of 6 doses versus 3 doses of Auxora in randomized patients with severe hypoxemic respiratory failure: those on HFNC with a recorded worst imputed PaO2/FiO2≤100 between the end of the infusion of Auxora at 24 hours and the start of the infusion of Auxora at 48 hours or those on NIV or IMV at the start of the infusion of Auxora at 48 hours.

### Treatment of Patients

All patients enrolled in the study should receive care consistent with local standard of care. Patients with worsening respiratory failure should receive conservative intravenous fluid strategies such as FACTT LITE. All patients should receive pharmacological prophylaxis to prevent the development of venous thromboembolic disease. The type and dose of prophylaxis should be determined by local standard of care.

Patients enrolled in the study should receive dexamethasone, or equivalent dose of another corticosteroid, as standard of care. If patients are not receiving dexamethasone at the time of enrollment into CARDEA-Plus, dexamethasone or the equivalent dose of another steroid should be started. If the patient is already receiving dexamethasone at the time of enrollment, dexamethasone should be continued on its established dosing schedule. The COVID-19 Treatment Guidelines Panel of the National Institutes of Health recommends using dexamethasone (at a dose of 6 mg per day, given orally or intravenously, for up to 10 days) in patients with COVID-19 who require supplemental oxygen. Of note, the dose equivalencies for dexamethasone 6 mg daily are prednisone 40 mg daily, methylprednisolone 32 mg daily, and hydrocortisone 160 mg daily.

If patients are not receiving remdesivir at the time of enrollment into CARDEA-Plus, starting remdesivir during the hospitalization may be considered. If the patient is already receiving remdesivir at the time of enrollment, remdesivir should be continued on its established dosing schedule. The suggested dose of remdesivir for adults weighing ≥40 kg and not requiring invasive mechanical ventilation and/or ECMO is a single dose of 200 mg infused intravenously over 30 to 120 minutes on Day 1 followed by once-daily maintenance doses of 100 mg infused intravenously over 30 to 120 minutes for 4 days (days 2 through 5). If a patient does not demonstrate clinical improvement, treatment may be extended for up to 5 additional days (i.e., up to a total of 10 days). Auxora and remdesivir should not infuse at the same time but should be given sequentially but may be given in any order.

For patients who within 3 days of hospitalization have rapidly increasing oxygen needs and evidence for systemic inflammation, the administration of tocilizumab, 8 mg/kg actual body weight (up to 800 mg) administered as a single IV dose, may be considered. Although approximately a third of patients in the REMAP-CAP and RECOVERY trials received a second dose of tocilizumab at the discretion of the treating physicians, data on outcomes based on receipt of one or two doses is not available. Therefore, there is insufficient evidence to determine which patients, if any, would benefit from an additional dose of tocilizumab.

Other immunosuppressive medications or immunotherapies are prohibited in patients enrolled into the study are listed below. The use of dextromethorphan is discouraged in patients enrolled into the study.

Prohibited medications: Any medication, with the exception of those listed below, may be given at the discretion of the PI. Medications that should not be administered during the study to patients enrolled in the study include:
Chemotherapy
Cyclosporine, Tacrolimus
Sirolimus, Everolimus
Azathioprine
Cyclosphosphamide
Methotrexate
Mycophenolate
Leflunomide
Biologics/Monoclonals: abatacept, adalimumab, alemtuzumab, anakinra, basilizimab, belimumab, bevacizumab, brodalumab, canakinumab, certolizumab, cetuximab, clazakizumab, daclizumab, eculizumab, etanercept, golimumab, infliximab, interferon, ixekizumab, muromonab, natalizumab, omalizumab, rituximab, sarilumab, secukinumab, trastuzumab, ustekinumab, vedolizumab
Baricitinib, Tofacitinib, or other Janus Kinase inhibitors

Note further use of Dextromethorphan is discouraged.

Discharge criteria: All patients should remain in the hospital until all 3 doses of Auxora have been administered. All randomized patients should remain in the hospital until the 3 additional doses of study drug have been administered.

If the patient is ready to be discharged before all doses of Auxora or study drug have been administered, the PI or treating physician should contact the Medical Monitor prior to discharging the patient.

Compliance: Only the PI or his/her appropriately trained study staff will administer study drug to patients enrolled in the trial in accordance with the protocol. Study drug must not be used for any reasons other than that described in the protocol.

Enrollment and randomization procedures: All enrolled patients will receive three doses of Auxora. Patients on HFNC with a recorded worst imputed PaO2/FiO2 ≤100 between the end of the infusion of Auxora at 24 hours and the start of the infusion of Auxora at 48 hours, or are on NIV or IMV at the start of the infusion of Auxora at 48 hours will be randomized 1:1 to receive three additional doses of study drug, either Auxora or volume matched Placebo. Patients on IMV will be stratified between the Auxora and Placebo groups. Enrollment and randomization will occur through a web-based system.

Discontinuation and withdrawal: The term discontinuation refers to a patient or PI discontinuing the administration of Auxora before all 3 doses are administered, or before the additional three doses of study drug are administered in randomized patients, despite the patient remaining in the hospital. Patients who do not receive all 3 doses of Auxora because the treating physician discharged them from the hospital because they have rapidly improved will not be considered to have discontinued study medication.

Patients have the right to discontinue the administration of Auxora, or study drug for randomized patients, at any time for any reason, without prejudice to their medical care. The PI may discontinue the administration of Auxora or study drug because of an adverse event or change in medical status that raises a safety concern about the patient receiving additional doses of Auxora or study drug. If possible, the PI should contact the Medical Monitor to review the reasons for a patient's discontinuation from Auxora or study drug. The PI should also record the reason for the discontinuation in the eCRF and appropriate source documents at the site. Even if the patient discontinues receiving Auxora or study drug, diligence should be taken to ensure that all study visits and assessments are completed through the end of the study.

Withdrawal refers only to the complete withdrawal of the patient from the study because of the withdrawal of consent. Patients should be offered discontinuation of Auxora or study drug as an alternative to withdrawal of consent to ensure they are followed for safety assessments. The PI should inform the Medical Monitor of the withdrawal of consent and record the withdrawal of consent in the eCRF and appropriate source documents at the site. For any patient that withdraws consent, the Investigator should endeavor (within the limits of privacy laws or other regulations) to report their vital status (e.g. dead or alive) at Day 30 and Day 60. Review of publicly available records, such as a death registers and/or information available on the Internet may be a suitable publicly available source.

### Drug Materials and Management

Auxora is to be administered as an IV infusion and is supplied as a translucent, white to yellowish, sterile, non-pyrogenic emulsion containing 1.6 mg/mL of the active pharmaceutical ingredient CM4620. Auxora is supplied as an 80 mL fill in a 100 mL single-use glass vial. Auxora contains egg phospholipids, medium chain triglycerides, glycerin, edetate disodium salt dehydrate (EDTA), sodium hydroxide (as needed to adjust pH), and sterile water for injection (Table 7).

TABLE 7
Auxora Product Information

| | |
|---|---|
| Product Name: | CM4620 Injectable Emulsion |
| Dosage Forms | Injectable Emulsion (Liquid) |
| Concentration of CM4620 | 1.6 mg/mL |
| Route of Administration | IV |
| Physical Description | Translucent, non-separated, white to |
| | yellowish emulsion |
| Inactive Ingredients | Sterile Water for Injection USP, Egg |
| | Phospholipid NF (80% Phosphatidylcholine), |
| | Medium Chain Triglycerides NP, Glycerin |
| | USP, and Edetate Disodium Salt Dihydrate |
| | (EDTA) USP. Sodium Hydroxide and |
| | Hydrochloric Acid may be added to adjust |
| | the pH. |
| Manufacturer | Bioserv Corporation |
| | San Diego, CA 92121 |

Matching Placebo is to be administered as an IV infusion and is supplied as a translucent, white to yellowish, sterile, non-pyrogenic emulsion carrier containing no active pharmaceutical ingredient. Placebo is supplied as an 80 mL fill in a 100 mL single-use vial. Placebo contains the same ingredients as Auxora except that it does not contain CM4620.

Auxora and Placebo must be maintained in a secure location with refrigerated temperature conditions of 2 to 8° C. (36 to 46° F.). Precaution should be taken to ensure that the Auxora and Placebo do not freeze. Temperature logs should be maintained and available during monitor review. When a temperature is noted outside the range of 2° C. to 8° C. lasting for 24 hours or more, or if the temperature exceeds 20° C. (68° F.), or is below 0° C. (32° F.), CalciMedica or its designee must be notified as soon as possible. The stability of Auxora and Placebo has been demonstrated to 24 months and is being evaluated for longer periods in ongoing studies. The Pharmacy Manual will also contain details regarding Auxora and Placebo storage in addition to procedures for managing and reporting temperature excursions.

The study pharmacist and/or designee will be responsible for the preparation and dispensation of Auxora and Placebo. Prior to administration, Auxora and Placebo both must be transferred to a sterile container a using sterile technique. Specific details on how to prepare Auxora and Placebo, as well as the specific components that will be used to administer both Auxora and Placebo, will be provided in the Pharmacy Manual. The Pharmacy Manual will also contain tables detailing the selected dose level and volume of administration of Auxora and Placebo.

Both Auxora and Placebo will be administered intravenously over 4 hours at a constant rate of infusion. Auxora will be administered every 24 hours (±1 hours) for three consecutive days for a total of 3 doses in all patients; randomized patients will receive either three additional doses or Auxora or Placebo. The dose and volume of Auxora, and the volume of Placebo, that will be administered will be calculated using the patient weight obtained at the time of hospitalization or during screening. A line into a peripheral or central vein may be used for the infusion. The peripheral IV should be 20 gauge in size or larger. The peripheral IV or central line port should be dedicated when administering Auxora or Placebo other than 0.9% normal saline. Auxora and Placebo are compatible with 0.9% normal saline. The IV tubing used to administer Auxora and Placebo must contain a 1.2 micron filter. The Pharmacy Manual will contain a recommended procedure to prime the IV tubing and flush the tubing, but this may be adapted to local nursing standards. 0.9% normal saline may be used to clear the line to ensure that the volume to be infused (VTBI) is completely administered. If the administration of Auxora or Placebo is stopped because of a technical reason, such as failure of the IV site, or IV pump malfunction, the administration of Auxora or Placebo should be resumed when the technical reason is resolved, and continued at the same rate until the infusion is completed. The total amount of time for the start of infusion to end of infusion of Auxora or Placebo should be recorded.

The study may be modified at any time the administered doses of Auxora or volumes of Placebo, the days of infusion, the timing of the infusion and the rate of infusion based on review of the safety and tolerability data by the IDMC. If the administration of Auxora or Placebo is stopped because of a serious adverse event that is considered to be probably or definitely related to Auxora or Placebo, the Medical Monitor must be immediately contacted.

Although the administration of the infusion should be set up to be completed over four hours, it is expected that there will be minor variability based on the equipment used and calibration of the equipment. The recommended infusion timeframe is 4 hours (±30 minutes). Infusion outside this timeframe will be evaluated to confirm that the full dose (≥90%) was administered. If the full dose was not administered, a protocol deviation will be recorded.

1. A method for treating acute lung injury (ALI), acute respiratory distress syndrome (ARDS), COVID-19 pneumonia, bacterial pneumonia, viral pneumonia, acute pancreatitis, or a combination thereof, in a subject, comprising administering a therapeutically effective amount of an intracellular Calcium signaling inhibitor to said subject, wherein the subject has a PaO₂/FiO₂ (P/F) ratio of less than or equal to about 300, and wherein the subject has been administered with at least two doses of the intracellular Calcium signaling inhibitor.

2. The method of claim 0, wherein the subject has a P/F ratio of less than or equal to about 125.

3. The method of claim 1, wherein the subject has a P/F ratio of less than or equal to about 100.

4. The method of claim 1, wherein the subject has a P/F ratio of about 75 to about 150.

5. (canceled)

6. The method of claim 1, wherein the subject shows increase in the P/F ratio after administration of the intracellular Calcium signaling inhibitor.

7.-14. (canceled)

15. The method of claim 1, wherein the intracellular Calcium signaling inhibitor is administered with at least three doses.

16. The method of claim 0, wherein a second dose of the intracellular Calcium signaling inhibitor is administered about 24 hours after a first dose and a third dose of the intracellular Calcium signaling inhibitor is administered about 48 hours after a first dose.

17. (canceled)

18. The method of claim 0, wherein an amount of the first dose is greater than the concentration of the second and third dose.

19.-24. (canceled)

25. The method of claim 1, wherein the ALI or ARDS comprises pneumonia.

26. (canceled)

27. (canceled)

28. The method of claim 25, wherein the pneumonia comprises a viral pneumonia due to a coronavirus, an adenovirus, an influenza virus, a rhinovirus, or a respiratory syncytial virus.

29. (canceled)

30. (canceled)

31. The method of claim 28, wherein the coronavirus is SARS-CoV, SARS-CoV-2, or MERS-CoV.

32.-34. (canceled)

35. The method of claim 1, wherein the intracellular Calcium signaling inhibitor is N-(5-(6-ethoxy-4-methylpyridin-3-yl)pyrazin-2-yl)-2,6-difluorobenzamide, N-(5-(2-ethyl-6-methylbenzo[d]oxazol-5-yl) pyridin-2-yl)-3,5-difluoroisonicotinamide, N-(4-(1-ethyl-3-(thiazol-2-yl)-1H-pyrazol-5-yl)phenyl)-2-fluorobenzamide, N-(5-(1-ethyl-3-(triflouromethyl)-1H-pyrazol-5-yl)pyrazin-2-yl)-2,4,6-trifluorobenzamide, 4-chloro-1-methyl-N-(4-(1-methyl-3-(trifluoromethyl)-1H-pyrazol-5-yl)phenyl)-1H-pyrazole-5-carboxamide, N-(4-(3-(difluoromethyl)-5-methyl-1H-pyrazol-1-yl)-3-fluorophenyl)-2,6-difluorobenzamide, N-(4-(3-(difluoromethyl)-5-methyl-1H-pyrazol-1-yl)-3-fluorophenyl)-2,4,6-trifluorobenzamide, N-(4-(3-(difluoromethyl)-1-methyl-1H-pyrazol-5-yl)-3-fluorophenyl)-2,4,6-trifluorobenzamide, 4-chloro-N-(3-fluoro-4-(1-methyl-3-(trifluoromethyl)-1H-pyrazol-5-yl)phenyl)-1-methyl-1H-pyrazole-5-carboxamide, 3-fluoro-4-(1-methyl-3-(trifluoromethyl)-1H-pyrazol-5-yl)-N-((3-methylisothiazol-4-yl)methyl) aniline, N-(5-(7-chloro-2,3-dihydro-[1,4]dioxino[2,3-b]pyridin-6-yl) pyridin-2-yl)-2,6-difluorobenzamide, N-(2,6-difluorobenzyl)-5-(1-ethyl-3-(thiazol-2-yl)-1H-pyrazol-5-yl)pyrimidin-2-amine, 3,5-difluoro-N-(3-fluoro-4-(3-methyl-1-(thiazol-2-yl)-1H-pyrazol-4-yl)phenyl) isonicotinamide, 5-(1-methyl-3-(trifluoromethyl)-1H-pyrazol-5-yl)-N-(2,4,6-trifluorobenzyl)pyridin-2-amine, N-(5-(1-ethyl-3-(trifluoromethyl)-1H-pyrazol-5-yl) pyridin-2-yl)-2,4,6-trifluorobenzamide, N-(5-(5-chloro-2-methylbenzo[d]oxazol-6-yl)pyrazin-2-yl)-2,6-difluorobenzamide, N-(5-(6-ethoxy-4-methylpyridin-3-yl) thiazol-2-yl)-2,3,6-trifluorobenzamide, N-(5-(6-ethoxy-4-methylpyridin-3-yl) thiazol-2-yl)-2,6-difluorobenzamide, N-(5-(1-ethyl-3-(trifluoromethyl)-1H-pyrazol-5-yl) pyridin-2-yl)-2,3,6-trifluorobenzamide, 2,3,6-trifluoro-N-(3-fluoro-4-(1-methyl-3-(trifluoromethyl)-1H-pyrazol-5-yl)phenyl)benzamide, 2,6-difluoro-N-(4-(5-methyl-2-(trifluoromethyl) oxazol-4-yl)phenyl)benzamide, 2,6-difluoro-N-(5-(2-methylbenzo[d]oxazol-6-yl)pyrazin-2-yl)benzamide, N-(4-(1-ethyl-3-(thiazol-2-yl)-1H-pyrazol-5-yl)phenyl)-2-fluorobenzamide, N-(4-(2-((6-chloropyridin-3-yl)oxy)-4-methylthiazol-5-yl)phenyl)-2-fluorobenzamide, N-(5-(2,5-dimethylbenzo[d]oxazol-6-yl) thiazol-2-yl)-2,3,6-trifluorobenzamide, or N-(5-(6-chloro-2,2-difluorobenzo[d][1,3]dioxol-5-yl)pyrazin-2-yl)-2-fluoro-6-methylbenzamide, or a pharmaceutically acceptable salt, pharmaceutically acceptable solvate, or pharmaceutically acceptable prodrug thereof.

36.-38. (canceled)

39. A method for treating acute lung injury (ALI), acute respiratory distress syndrome (ARDS), COVID-19 pneumonia, bacterial pneumonia, viral pneumonia, acute pancreatitis, or a combination thereof, in a subject, comprising administering a therapeutically effective amount of a corticosteroid and/or immunosuppressive drug and an intracellular Calcium signaling inhibitor to said subject, wherein the subject has a PaO₂/FiO₂ (P/F) ratio of less than or equal to about 300.

40.-45. (canceled)

46. The method of claim 39, wherein the subject shows increase in the P/F ratio after administration of the intracellular Calcium signaling inhibitor.

47.-54. (canceled)

55. The method of claim 39, wherein the intracellular Calcium signaling inhibitor is administered with at least three doses.

56.-64. (canceled)

65. The method of claim 39, wherein the ALI or ARDS comprises pneumonia.

66. (canceled)

67. (canceled)

68. The method of claim 65, wherein the pneumonia comprises a viral pneumonia due to a coronavirus, an adenovirus, an influenza virus, a rhinovirus, or a respiratory syncytial virus.

69. (canceled)

70. (canceled)

71. The method of claim 68, wherein the coronavirus is SARS-CoV, SARS-CoV-2, or MERS-CoV.

72-74. (canceled)

75. The method of claim 39, wherein the intracellular Calcium signaling inhibitor is N-(5-(6-ethoxy-4-methylpyridin-3-yl)pyrazin-2-yl)-2,6-difluorobenzamide, N-(5-(2-ethyl-6-methylbenzo[d]oxazol-5-yl) pyridin-2-yl)-3,5-difluoroisonicotinamide, N-(4-(1-ethyl-3-(thiazol-2-yl)-1H-pyrazol-5-yl)phenyl)-2-fluorobenzamide, N-(5-(1-ethyl-3-(triflouromethyl)-1H-pyrazol-5-yl)pyrazin-2-yl)-2,4,6-trifluorobenzamide, 4-chloro-1-methyl-N-(4-(1-methyl-3-(trifluoromethyl)-1H-pyrazol-5-yl)phenyl)-1H-pyrazole-5-carboxamide, N-(4-(3-(difluoromethyl)-5-methyl-1H-pyrazol-1-yl)-3-fluorophenyl)-2,6-difluorobenzamide, N-(4-(3-(difluoromethyl)-5-methyl-1H-pyrazol-1-yl)-3-fluorophenyl)-2,4,6-trifluorobenzamide, N-(4-(3-(difluoromethyl)-1-methyl-1H-pyrazol-5-yl)-3-fluorophenyl)-2,4,6-trifluorobenzamide, 4-chloro-N-(3-fluoro-4-(1-methyl-3-(trifluoromethyl)-1H-pyrazol-5-yl)phenyl)-1-methyl-1H-pyrazole-5-carboxamide, 3-fluoro-4-(1-methyl-3-(trifluoromethyl)-1H-pyrazol-5-yl)-N-((3-methylisothiazol-4-yl)methyl) aniline, N-(5-(7-chloro-2,3-dihydro-[1,4]dioxino[2,3-b]pyridin-6-yl) pyridin-2-yl)-2,6-difluorobenzamide, N-(2,6-difluorobenzyl)-5-(1-ethyl-3-(thiazol-2-yl)-1H-pyrazol-5-yl)pyrimidin-2-amine, 3,5-difluoro-N-(3-fluoro-4-(3-methyl-1-(thiazol-2-yl)-1H-pyrazol-4-yl)phenyl) isonicotinamide, 5-(1-methyl-3-(trifluoromethyl)-1H-pyrazol-5-yl)-N-(2,4,6-trifluorobenzyl)pyridin-2-amine, N-(5-(1-ethyl-3-(trifluoromethyl)-1H-pyrazol-5-yl) pyridin-2-yl)-2,4,6-trifluorobenzamide, N-(5-(5-chloro-2-methylbenzo[d]oxazol-6-yl)pyrazin-2-yl)-2,6-difluorobenzamide, N-(5-(6-ethoxy-4-methylpyridin-3-yl) thiazol-2-yl)-2,3,6-trifluorobenzamide, N-(5-(6-ethoxy-4-methylpyridin-3-yl) thiazol-2-yl)-2,6-difluorobenzamide, N-(5-(1-ethyl-3-(trifluoromethyl)-1H-pyrazol-5-yl) pyridin-2-yl)-2,3,6-trifluorobenzamide, 2,3,6-trifluoro-N-(3-fluoro-4-(1-methyl-3-(trifluoromethyl)-1H-pyrazol-5-yl)phenyl)benzamide, 2,6-difluoro-N-(4-(5-methyl-2-(trifluoromethyl) oxazol-4-yl)phenyl)benzamide, 2,6-difluoro-N-(5-(2-methylbenzo[d]oxazol-6-yl)pyrazin-2-yl)benzamide, N-(4-(1-ethyl-3-(thiazol-2-yl)-1H-pyrazol-5-yl)phenyl)-2-fluorobenzamide, N-(4-(2-((6-chloropyridin-3-yl)oxy)-4-methylthiazol-5-yl)phenyl)-2-fluorobenzamide, N-(5-(2,5-dimethylbenzo[d]oxazol-6-yl) thiazol-2-yl)-2,3,6-trifluorobenzamide, or N-(5-(6-chloro-2,2-difluorobenzo[d][1,3]dioxol-5-yl)pyrazin-2-yl)-2-fluoro-6-methylbenzamide, or a pharmaceutically acceptable salt, pharmaceutically acceptable solvate, or pharmaceutically acceptable prodrug thereof.

76.-78. (canceled)

79. The method of claim 39, wherein the corticosteroid is cortisol, cortisone, prednisone, prednisolone, methylprednisolone, dexamethasone, betamethasone, triamcinolone, fludrocortisone, fludrocortisone acetate, deoxycorticosterone, deoxycorticosterone acetate, aldosterone, beclomethasone, or progesterone.

80. (canceled)

81. The method of claim 39, wherein the immunosuppressive drug is a corticosteroid, a glucocorticoid, an anti-IL-6, an immunomodulatory imide drug, 4-deoxypyridoxine, fingolimod, laquinimod, mitzoribine, mycophenolic acid, pimecrolimus, tocilizumab or voclosporin.

82. (canceled)

83. The method of claim 1, wherein the subject has a P/F ratio of less than or equal to about 200.

84. The method of claim 1, wherein the ALI or ARDS comprises a respiratory failure.

85. The method of claim 84, wherein the respiratory failure is mild respiratory failure indicated by a P/F ratio of 200-300, moderate respiratory failure indicated by a P/F ratio of 100-200, or severe respiratory failure indicated by a P/F ratio of less than 100.

86. The method of claim 1, wherein a P/F ratio of less than 100 after two doses of the Calcium channel inhibitor indicates that the subject is a non-responder, wherein the subject further receives up to six total dose.

87. The method of claim 15, wherein the intracellular Calcium signaling inhibitor is administered up to six total doses.

88. The method of claim 39, wherein the ALI or ARDS comprises a respiratory failure.
